(19) United States
(12) Patent Application Publication
(10) Pub. No.: US 2004/0152740 A1
(43) Pub. Date: Aug. 5, 2004

(54) Arylglycine derivatives for use as glycine transport inhibitors

(75) Inventors: Methvin Isaac, Etobicoke (CA); Tao Xin, Woodbridge (CA); Tomislav Stefanac, Burlington (CA); Anne O'Brien, Toronto (CA); Kathleen Da Silva-Turcot, Toronto (CA); Jalaj Arora, Milton (CA); Shawn Maddaford, Mississauga (CA); Abdelmalik Slassi, Mississauga (CA)

Correspondence Address: JOHN W. RYAN, C/O DECHERT LLP, PRINCETON PIKE CORPORATION CENTER, P.O. BOX 5218, PRINCETON, NJ, 08543-5218, US

(73) Assignee: NPS Allelix Corporation, Mississauga (CA)

(21) Appl. No.: 10/657,812

(22) Filed: Sep. 8, 2003

Related U.S. Application Data
(60) Provisional application No. 60/409,420, filed on Sep. 9, 2002.

(51) Int. Cl. C07D 213/72; A61K 31/44; A61K 31/40; C07D 207/34; A61K 31/17

(52) U.S. Cl. 514/352; 514/426; 514/586; 546/305; 548/557; 564/27

(57) ABSTRACT

The present invention relates to compounds of Formula 1:

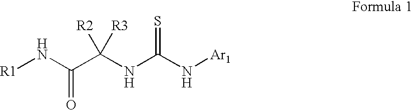

and salts solvates and hydrates thereof. The invention further relates to pharmaceutical compositions containing said compounds and methods of treating neurological and neuropsychistric disorders using said compounds.

This application claims priority to U.S. Provisional Application No. 60/409,420 filed Sep. 9, 2002.

The present invention relates to a class of compounds, to pharmaceutical compositions containing them and to methods of treating neurological and neuropsychiatric disorders using such compounds.

### BACKGROUND OF THE INVENTION

Synaptic transmission is a complex form of intercellular communication that involves a considerable array of specialized structures in both the pre- and post-synaptic terminal and surrounding glial cells (Kanner and Schuldiner, CRC Critical Reviews in Biochemistry, 22, 1987:1032). Transporters sequester neurotransmitters from the synapse, thereby regulating the concentration of neurotransmitters in the synapse, and their duration therein, which together influence the magnitude of synaptic transmission. Further, by preventing the spread of neurotransmitters to neighbouring synapses, transporters maintain the fidelity of synaptic transmission. Lastly, by sequestering released neurotransmitter into the presynaptic terminal, transporters allow for neurotransmitter re-utilization.

Neurotransmitter transport is dependent upon extracellular sodium and the voltage difference across the membrane; under conditions of intense neuronal firing, as, for example, during a seizure, transporters can function in reverse, releasing neurotransmitter in a calcium-independent non-exocytotic manner (Attwell et al., Neuron, 11, 1993:401-407). Pharmacologic modulation of neurotransmitter transporters thus provides a means for modifying synaptic activity, which provides useful therapy for the treatment of neurological and psychiatric disturbances.

The amino acid glycine is a major neurotransmitter in the mammalian central nervous system, functioning at both inhibitory and excitatory synapses. By nervous system, both the central and peripheral portions of the nervous system are intended. These distinct functions of glycine are mediated by two different types of receptor, the glycine receptor and the NMDA receptor, each of which is associated with a different class of glycine transporter.

The inhibitory actions of glycine are mediated by glycine receptors that are sensitive to the convulsant alkaloid strychnine, and are thus referred to as “strychnine-sensitive”. Such receptors contain an intrinsic chloride channel that is opened upon binding of glycine to the receptor; by increasing chloride conductance, the threshold for firing of an action potential is increased. Strychnine-sensitive glycine receptors are found predominantly in the spinal cord and brainstem, and pharmacological agents that enhance the activation of such receptors will thus increase inhibitory neurotransmission in these regions.

Glycine also functions in excitatory transmission by modulating the actions of glutamate, the major excitatory neurotransmitter in the central nervous system. See Johnson and Ascher, Nature, 325, 1987:529-531; Fletcher et al., Glycine Transmission, Otterson and Storm-Mathisen, eds., 1990:193-219. Specifically, glycine is an obligatory co-agonist at the class of glutamate receptor termed N-methyl-D-aspartate (NMDA) receptor. Activation of NMDA receptors increases sodium and calcium conductance, which depolarizes the neuron, thereby increasing the likelihood that it will fire an action potential. NMDA receptors are widely distributed throughout the brain, with a particularly high density in the cerebral cortex and hippocampal formation.

Molecular cloning has revealed the existence in mammalian brains of two classes of glycine transporters, termed GlyT-1 and GlyT-2. GlyT-1 is found throughout the brain and spinal cord, and it has been suggested that its distribution corresponds to that of glutamatergic pathways and NMDA receptors (Smith, et al., Neuron, 8, 1992:927-935). Molecular cloning has further revealed the existence of four variants of GlyT-1, termed GlyT-1a, GlyT-1b GlyT-1c and GlyT-1 d. Two of these variants (1a and 1b) are found in rodents, each of which displays a unique distribution in the brain and peripheral tissues (Borowsky et al., Neuron, 10, 1993:851-863; Adams et al., J. Neuroscience, 15, 1995:2524-2532). The third variant, 1 c, has only been detected in human tissues (Kim, et al., Molecular Pharmacology, 45, 1994:608-617). The fourth variant has been detected in human tissue (see U.S. Pat. No. 6,008,015). These variants arise by differential splicing and exon usage, and differ in their N-terminal regions. GlyT-2, in contrast, is found predominantly in the brain stem and spinal cord, and its distribution corresponds closely to that of strychnine-sensitive glycine receptors (Liu et al., J. Biological Chemistry, 268, 1993:22802-22808; Jursky and Nelson, J. Neurochemistry, 64, 1995:1026-1033). Another distinguishing feature of glycine transport mediated by GlyT-2 is that it is not inhibited by sarcosine as is the case for glycine transport mediated by GlyT-1.

For the above reasons it is the view that, by regulating the synaptic levels of glycine, GlyT-1 and GlyT-2 selectively influence the activity of NMDA receptors and strychnine-sensitive glycine receptors, respectively.

Compounds which inhibit or activate glycine transporters would thus be expected to alter the glycine concentration and thereby alter glycine receptor activation and, thus, provide therapeutic benefits in a variety of disease states. Inhibition of GlyT-2, for example, may be used to increase the activity of inhibitory neurons having strychnine-sensitive glycine receptors via increasing synaptic levels of glycine, thus diminishing the transmission of pain-related (i.e. nociceptive) information in the spinal cord, which has been shown to be mediated by these receptors (Yaksh, Pain, 37, 1989:111-123). Additionally, enhancing inhibitory glycinergic transmission through strychnine-sensitive glycine receptors in the spinal cord may be used to decrease muscle hyperactivity, which may be useful in treating diseases or conditions associated with increased muscle contraction, such as spasticity (Truong et al., Movement Disorders, 3, 1988:77-89; Becker, FASEB J, 4, 1990:2767-2774). Spasticity associated with stroke, head trauma, multiple sclerosis, spinal cord injury, dystonia, and other conditions of illness and injury of the nervous system (such as epilepsy) may be treated via modulation of glycine transporters.

### SUMMARY OF THE INVENTION

According to one aspect of the invention there are described compounds of Formula 1 as described below:

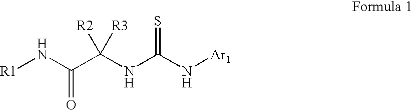

wherein;

R₁ is selected from the group consisting of aryl, heteroaryl, cylcloalkyl and heterocycloalkyl;

wherein R₁ is optionally substituted with one or more substituents Rₐ;

wherein Rₐ is selected from the group consisting of alkyl, alkoxy, halo, cyano, alkanoyl, haloalkyl, thioalkyl, nitro, aryl, heteroaryl, aralkyl, heteroaralkyl and —(R₇)ₙNR₈R₉, wherein R₇ is selected from alkyl, alkoxy, and oxyalkyl, R₈ and R₉ can be independently selected from H, and alkyl, or R₈ and R₉ can join together such that NR₈R₉ form a 5 or 6-member heterocyclic ring, and n is selected from 0, 1, 2 and 3,

wherein the substituent(s) Rₐ is optionally further substituted with one or more substituents selected from the group consisting of alkyl, alkoxy, halo, cyano, alkanoyl, haloalkyl, thioalkyl, nitro, and —(R₇)ₙNR₈R₉, wherein R₇, R₈, R₉ and n are as defined above;

R₂ and R₃ are:

a) independently selected from the group consisting of H, alkyl, aralkyl, optionally substituted aryl, optionally substituted heteroaryl, and optionally substituted, saturated or unsaturated, 5-or 6-membered, homocyclic or heterocyclic rings wherein the optional substituent may be selected from the group consisting of H, alkyl, alkoxy and halo; or

b) joined together to form a 3, 4, 5, 6, or 7 member spirocyclic ring;

Ar₁ is aryl; and

Ar₁ is optionally substituted with one or more substituents R_{b};

wherein R_{b} is selected from the group consisting of: alkyl, alkoxy, halo, haloalkyl, nitro, —(R₇)ₙNR₈R₉, alkanoyl, aryl, heteroaryl, —O(CH₂)ₘNR₁₀R₁₁ and —SO₂—NR₁₀R₁₁ (wherein R₇, R₈, R₉ and n are as described above, and the groups R₁₀ and R₁₁ can be independently selected from H, or alkyl, or R₁₀ and R₁₁ can join together such that NR₁₀R₁₁ form a 5 or 6-member heterocyclic ring and m is selected from 1, 2, 3, 4, and 5);

wherein the substituent(s) R_{b} is optionally further substituted with one or more substituents, selected from the group consisting of alkyl, alkoxy, halo, cyano, alkanoyl, haloalkyl, thioalkyl, nitro and —(R₇)ₙNR₈R₉, wherein R₇, R₈, R₉, and n are as described above;

wherein when Ar₁ is phenyl then

a) Ar₁ has a substituent R_{b} at the 2-position wherein the substituent is selected from the group consisting of: nitro; haloalkyl; cyano; —C(O)R₁₂; —C(O)OR₁₂; —C(O)NR₁₂R₁₃; —S(O)R₁₂; —S(O)₂R₁₂ and —S(O)₂NR₁₂R₁₃, wherein R₁₂ and R₁₃ are independently selected from H and alkyl, or

b) Ar₁ has an alkanoyl substituent R_{b} at the 4-position;

and a salt solvate or hydrate thereof.

In another aspect of the invention, compounds of the invention inhibit glycine transport via the GlyT-2 transporter. By GlyT-2 we mean those glycine transporters found predominantly in the brain stem and spinal cord and the distribution of which corresponds closely to that of strychnine-sensitive glycine receptors (Liu et al. J. Biological Chemistry, 268, 1993:22802-22808; Jursky and Nelson, J. Neurochemistry, 64, 1995:1026-1033), and as described in U.S. Pat. No. 5,700,013.

According to another aspect of the invention, there is provided a pharmaceutical composition comprising a compound of Formula I in an amount effective to inhibit glycine transport, and a pharmaceutically acceptable carrier.

In another aspect of the present invention, there are provided compositions containing the present compounds in amounts suitable for pharmaceutical use to treat medical conditions for which a glycine transport inhibitor is indicated, such as the treatment of pain, epilepsy or conditions associated with increased muscle contraction such as spasticity and spasticity associated with stroke, head trauma, multiple sclerosis, spinal cord injury and dystonia.

These and other aspects of the present invention are described in greater detail herein below.

Definitions

The term “aryl” as used herein means a 5, 6, 7, 8, 9 or 10 member monocyclic, bicyclic or benzo-fused aromatic group such as phenyl, naphthyl, indanyl, tetrahydronaphthyl, dihydronaphthyl, indenyl, and the like.

The term “heteroatom” as used herein means a non-carbon atom such as S, N, O and the like.

The term “heteroaryl” as used herein means an aryl group containing 1, 2, 3 or 4 heteroatoms selected from N, O and S with the proviso that no two like heteroatoms are adjacent unless both are N, and includes such compounds as pyridyl, furyl, thienyl, pyrimidinyl, pyrollyl, imidazolyl, triazolyl, thiazolyl, oxazolyl, thiadiazolyl, oxadiazolyl, quinolinyl, quinoxylinyl, quinazolinyl, pyrazinyl, pyrimidinyl, indolyl, indazolyl, azaindolyl, isoquinolyl and the like.

The term “alkyl” as used herein means straight- and branched-chain alkyl radicals containing 1, 2, 3, 4, 5 or 6 carbon atoms and includes methyl, ethyl, propyl, isopropyl, butyl, i-butyl, s-butyl, t-butyl, pentyl, i-pentyl, t-pentyl, neopentyl, hexyl, and the like.

The term “cycloalkyl” as used herein means a carbocyclic ring containing 3, 4, 5, 6, 7 or 8 carbon atoms and includes cyclopropyl, cyclopentyl, cyclohexyl, cylcoheptyl, and cyclooctyl and the like.

The term “heterocycloalkyl” as used herein means a 3, 4, 5, 6, 7 or 8-membered ring containing one or two heteroatoms selected from the group consisting of N, S, and O and includes piperidinyl, piperazinyl, tetrahydropyran, tetrahydrothiopyran, morpholine, thiomorpholine, tetrahydrofuran, tetrahydrothiophene and the like.

The term “alkoxy” as used herein means straight- and branched-chain alkoxy radicals containing 1, 2, 3, 4, 5 or 6 carbon atoms and includes methoxy, ethoxy propoxy, isopropoxy, butoxy, tertbutoxy, pentoxy, hexloxy and the like.

The terms “aralkyl” as used herein means an alkyl radical as previously described substituted with an aryl group as previously described and includes benzyl, phenethyl and the like.

The term “aralkoxy” as used herein means an alkoxy radical substituted with an aryl group such as benzyloxy, phenethyloxy and the like.

The term “aryloxy” as used herein means an aryl substituted oxy radical such as phenoxy.

The terms “alkylene”, “alkenylene” and “alkynylene” as used herein means straight- and branched-chain bivalent radicals containing from one to six carbon atoms, such as methylene, ethylene, vinylene, propenylene and ethynylene.

The term “alkanoyl” as used herein means straight- and branched-chain radicals containing from 1, 2, 3, 4, 5 or 6 carbon atoms and includes acetyl, ethanoyl, propionyl, butanoyl, pentanoyl, hexanoyl and the like.

The term “halo” as used herein means halogen and includes fluoro, chloro, bromo and iodo.

The term “haloalkyl” as used herein means a straight or branched chain alkyl radical of 1, 2, 3, 4, 5 or 6 carbons with one or more halogen substituents such as trifluoromethyl, bromoethyl, chloromethyl, chlorohexyl and the like.

The term “thioalkyl” as used herein means straight- and branched-chain alkyl containing 1, 2, 3, 4, 5 or 6 carbons bonded through a sulfur radical and includes thiomethyl (CH₃S—), thioethyl, thiopropyl thiobutyl, thio-t-butyl, and the like.

The term “sulfonamido” as used herein means sulfonamide radicals where the nitrogen may be unsubstituted or substituted or a member of a ring and includes —S(O)₂NRR wherein R can be H, alkyl, alkoxy, cycloalkyl, aryl and the like or the R groups may join together such that NRR forms a ring.

The term “SPE tube” as used herein refers to a solid phase extraction tube. These may be commercially prepared disposable tubes filled with Silica gel for carrying out chromatography or “flash” chromatography (chromatography under pressure). Such tubes are purchased from Varian and Supelco.

The term “pharmaceutically acceptable salt” means an acid addition salt which is compatible with the treatment of patients.

A “pharmaceutically acceptable addition salt” is any non-toxic organic or inorganic acid addition salt of the base compounds represented by Formula 1 or any of Formula 1's intermediates. Illustrative inorganic acids which form suitable salts include hydrochloric, hydrobromic, sulfuric, and phosphoric acid and acid metal salts such as sodium monohydrogen, orthophosphate and potassium hydrogensulfate. Illustrative organic acids which form suitable salts include the mono-, di-, and tricarboxylic acids. Illustrative of such acids are for example acetic, glycolic, lactic, pyruvic, malonic, succinic, glutaric, fumaric, malic, tartaric, citric, ascorbic, maleic, hydromaleic, benzoic, hydroxybenzoic, phenylacetic, cinnamic, salicylic, 2-phenoxybenzoic, p-toluensulfonic acid and other sulfonic acids such as methanesulfonic acid and 2-hydroxyethanesulfonic acid. Either the mono- or di-acid salts can be formed and such salts can exist in either a hydrated, solvated, or substantially anhydrous form. In general, the acid addition salts of these compounds are more soluble in water and various hydrophilic organic solvents, and generally demonstrate higher melting points in comparison to their free base forms. The selection criteria for the appropriate salt will be known to one skilled in the art.

The term “solvate” as used herein means a compound of Formula 1 wherein molecules of a suitable solvent are incorporated in a crystal lattice. A suitable solvent is physiologically tolerable at the dosage administered as the solvate. Examples of suitable solvents are ethanol and the like.

The term “stereoisomers” is a general term for all isomers of the individual molecules that differ only in the orientation of their atoms in space. It includes mirror image isomers (enantiomers), geometric isomers (cis/trans) and isomers of compounds with more than one chiral centre that are not mirror images of one another (diastereomers).

The term “treat” or “treating” means to alleviate symptoms, or eliminate or reduce the causation of the symptoms, either on a temporary or permanent basis, or to prevent or slow the appearance of symptoms of the named disorder or condition.

The term “therapeutically acceptable carrier” means a non-toxic solvent, dispersant, excipient, adjuvant, or other material which is mixed with the active ingredient in order to permit the formation of a pharmaceutical composition, i.e., a dosage form capable of administration to the patient.

### DETAILED DESCRIPTION AND PREFERRED EMBODIMENTS

Included in the invention are compounds of Formula 1. In suitable embodiments of Formula 1, R₁ is selected from the group consisting of aryl, heteroaryl, cylcloalkyl and heterocycloalkyl, and R₁ is optionally substituted.

In a suitable embodiment of the invention R₁ is selected from the group described above and is optionally susbtituted with one or more substituents Rₐ, wherein Rₐ is selected from alkyl, alkoxy, halo, cyano, alkanoyl, haloalkyl, thioalkyl, nitro, aryl, heteroaryl, aralkyl, heteroaralkyl and —(R₇)ₙNR₈R₉, wherein R₇ is selected from alkyl, alkoxy, and oxyalkyl, R₈ and R₉ can be independently selected from H and alkyl, or R₈ and R₉ can join together such that NR₈R₉ form a 5 or 6-member heterocyclic ring or a heteroaryl ring, and n is selected from 0, 1, 2 and 3), wherein the substituent(s) Rₐ are optionally further substituted with one or more substituents selected from the group consisting of alkyl, alkoxy, halo, cyano, alkanoyl, haloalkyl, thioalkyl, nitro, and —(R₇)ₙNR₈R₉, wherein R₇, R₈, R₉ and n are as described above.

In another suitable embodiment of the invention R₁ is selected from optionally substituted phenyl, naphthyl, tetrahydonaphthyl, and pyridyl. In a particular embodiment, R₁ is pyridyl. In other embodiments R₁ is naphthyl or tetrahydronaphthyl. In still another embodiment R₁ is optionally substituted phenyl.

In a suitable embodiment of the invention R₁ is mono or di-substituted phenyl wherein the substituents, in order of preference, are located at the 2 and 6 positions>the 2 position>the 3 position>the 2 and 5 position>the 2 and 3 position>the 2 and 4 position>the 4 position.

In a further suitable embodiment of the invention R₁ is mono or di-substituted phenyl wherein the substituents are selected independently from alkyl, alkoxy, halo, cyano, alkanoyl, haloalkyl, thioalkyl, nitro, and piperazinyl.

In another suitable embodiment R₁ is mono or di-substituted phenyl with the substituents selected from methyl, ethyl, i-propyl, Cl, F, trifuoromethyl, CH₃S—, -cyano, nitro, methoxy, and piperazinyl.

In another embodiment R₁ is mono or di-substituted phenyl and the substituents are selected from methyl and ethyl.

In still another embodiment R₁ is phenyl and there is one substituent. In a particular embodiment the substituent is methyl and is located at the 2-position.

In yet another embodiment R₁ is phenyl and there are two substituents. In certain embodiments the substituents are located at the 2 and the 6 positions and the first is methyl and the second is selected from methyl or ethyl.

In a particular embodiment R₁ is 2-methylphenyl or R₁ is 2,6-dimethylphenyl.

In another embodiment of the invention there are compounds of Formula 1 wherein Ar₁ is optionally substituted aryl wherein the optional substituents are designated as R_{b} and are selected from the group consisting of alkyl, alkoxy, halo, haloalkyl, nitro, alkanoyl, aryl, heteroaryl, —(R₇)ₙNR₈R₉, wherein R₇, R₈, R₉ and n are as described above, O(CH₂)ₘNR₁₀ R₁₁, and —SO₂—NR₁₀R₁₁, wherein R₁₀ and R₁₁ can be independently selected from H and alkyl, or R₁₀ and R₁₁ can join together such that NR₁₀R₁₁ forms a 5 or 6-member heterocyclic ring. Further, the group(s) R_{b} can be substituted with one or more substituents selected from the group consisting of alkyl, alkoxy, halo, cyano, alkanoyl, haloalkyl, thioalkyl, nitro, and —(R₇)ₙNR₈R₉.

In a further embodiment of the invention Ar₁ is substituted phenyl with either a) a substituent R_{b} at the 2-position, wherein the substituent is selected from the group consisting of nitro, haloalkyl, cyano, —C(O)R₁₂, —C(O)OR₁₂, —C(O)NR₁₂R₁₃, —S(O)R₁₂, —S(O)₂R₁₂—S(O)₂NR₁₂R₁₃ (wherein R₁₂ and R₁₃ are independently selected from H and alkyl) or b) an alkanoyl substituent R_{b} at the 4-position.

In another embodiment of the invention Ar₁ is optionally substituted phenyl wherein the substituents are selected from alkyl, alkoxy, halo, haloalkyl, nitro, —(R₇)ₙNR₈R₉, alkanoyl, aryl, heteroaryl, —O(CH2)ₘNR₁₀R₁₁ and —SO₂—NR₁₀R₁₁ (wherein the groups are R₁₀ and R₁₁ can be independently selected from H and alkyl, or R₁₀ and R₁₁ can join together such that NR₁₀R₁₁ forms a 5 or 6-member ring, and m is selected from 1, 2, 3, 4 and 5) and any of these substituents may be further substituted with a group selected from alkyl, alkoxy, halo, cyano, alkanoyl, haloalkyl, thioalkyl, nitro and —(R₇)ₙNR₈R₉, wherein R₇, R₈, R₉ and n are as defined above.

In another suitable embodiment of the invention Ar₁ is mono or di-substituted phenyl wherein, in order of preference, the substituents are located at the 2 and 5 position>the 2 and 4 position>the 2 position>the 4 position.

In another suitable embodiment of the invention Ar₁ is mono or di-substituted phenyl wherein the substituents are selected from nitro, alkoxy, trifluoromethyl, F, acetyl, —O(CH₂)ₘNR₁₀R₁₁, and —SO₂—NR₁₀R₁₁ wherein R₁₀, R₁₁, and m are as defined above.

In still another embodiment of the invention Ar₁ is phenyl substituted at the 2 position wherein the substitutent is selected from nitro, trifluoromethyl and —SO₂—NR₁₀R₁₁.

In another embodiment Ar₁ is di-substituted phenyl wherein one substituent is selected from nitro, trifluromethyl and —SO₂—NR₁₀R₁₁, and the second substituent is selected from the group consisting of methoxy, ethoxy, propoxy, acetyl, and —O(CH₂)ₙNR₁₀R₁₁ wherein R₁₀, R₁₁, and n are as defined above.

In still a further embodiment Ar₁ is 2-nitrophenyl or 2-nitro-4-methoxyphenyl.

In another embodiment of the invention Ar₁ is naphthyl.

In another aspect of the invention R₂ and R₃ are independently selected from the group consisting of H, alkyl, aralkyl, haloalkyl, optionally substituted aryl, and optionally substituted heteroaryl and optionally substituted saturated or unsaturated 5 or 6-member homocyclic or heterocyclic rings. The optional substituent is selected from the group consisting of H, alkyl, alkoxy, and halo. Alternatively R₂ and R₃ are joined together to form a 3, 4, 5, 6 or 7 member spirocyclic ring.

In a certain embodiment of the invention R₂ is H and R₃ is selected from alkyl, aralkyl, optionally substituted aryl, optionally substituted heteroaryl and optionally substituted, saturated or unsaturated, 5-or 6-membered, homocyclic or heterocyclic. The optional substituents are selected from the group consisting of H, alkyl, alkoxy and halo.

In a further embodiment of the invention R₂ is H and R₃ is selected from methyl, isopropyl, t-Butyl, sec-Butyl, cyclohexyl, phenyl, benzyl, 3-thiophene.

In a particular embodiment of the invention R₂ is H and R₃ is phenyl.

In another embodiment of the invention R₂ and R₃ together form a 3, 5, or 6 membered spirocycle.

Specific embodiments of the invention include the following compounds of Formula 1:

N-(2-methylphenyl)-2-[3-(4-ethoxy-2-nitrophenyl)-thioureido]-2-phenyl acetamide (E4.3),

N-(2,6-dimethylphenyl)-2-[3-(4-methoxy-2-nitrophenyl)-thioureido]-2-phenyl acetamide (E33.6),

N-(2-methylphenyl)-2-[3-(2-nitrophenyl)-thioureido]-2-phenyl acetamide (E4.2),

N-(2-methylphenyl)-2-[3-(2-nitro-4-methoxyphenyl)-thioureido]-2-phenyl acetamide (E4.4),

N-(2,6-dimethylphenyl)-2-[3-(2-trifluoromethyl phenyl)-thioureido]-2-phenyl acetamide (E33.7),

N-(2,6-dimethylphenyl)-2-[3-(4-N,N-dimethylaminoethoxy-2-trifluoromethylphenyl)-thioureido]-2-phenyl acetamide (E33.8),

N-(2-isopropyl-6-methylphenyl)-2-[3-(4-methoxy-2-nitrophenyl)-thioureido]-2-phenyl acetamide (E28.1),

N-(2-chloro-6-methylphenyl)-2-[3-(4-methoxy-2-nitrophenyl)-thioureido]-2-phenyl acetamide (E29.1),

N-(2,6-dimethylphenyl)-2-[3-(4-methoxy-2-nitrophenyl)-thioureido]-4-methylpentanamide (E51.3),

N-(2,6-dimethylphenyl)-2-[3-(4-(2-N,N-dimethylamino)ethoxy-2-nitrophenyl)-thioureido]-2-phenyl acetamide (E33.4),

(R)-N-(2,6-dimethylphenyl)-2-[3-(4-methoxy-2-nitrophenyl)-thioureido]-4-methylpentanamide (E51.1*),

N-(2,6-dimethylphenyl)-2-[3-(4-ethoxy-2-nitrophenyl)-thioureido]-2-phenyl acetamide (E33.1),

N-(2,6-dimethylphenyl)-2-[3-(2-N,N-dimethylsulphonamidophenyl)-thioureido]-2-phenyl acetamide (E33.2),

N-(2,6-dimethylphenyl)-2-[3-(2-N-methylpiperizinylsulphonamidophenyl)-thioureido]-2-phenyl acetamide (E33.3)

and N-(2,6-dimethylphenyl)-2-[3-(4-(2-N,N-dimethylamino)sulphonamide-2-nitro-thioureido]-2-phenyl acetamide (E33.5).

In another embodiment of the invention, the compound of Formula 1 is provided in labeled form, such as a radiolabeled form, e.g. labeled by incorporation within its structure ³H or ¹⁴C or by conjugation to ¹²⁵I. In a preferred aspect of the invention, those compounds which bind preferentially to GlyT-2 versus GlyT-1 can be used, in labeled form, to identify GlyT-2 receptor ligands by techniques common in the art. This can be achieved by incubating the receptor or tissue in the presence of a ligand candidate and then incubating the resulting preparation with an equimolar amount of radiolabeled compound of the invention. GlyT-2 receptor ligands are thus revealed as those that significantly occupy the GlyT-2 site and prevent binding of the radiolabeled compound of the present invention. Alternatively, GlyT-2 receptor ligand candidates may be identified by first incubating a radiolabeled form of a compound of the invention then incubating the resulting preparation in the presence of the candidate ligand. A more potent GlyT-2 receptor ligand will, at equimolar concentration, displace the radiolabeled compound of the invention.

Acid addition salts of the compounds of Formula 1 are most suitably formed from pharmaceutically acceptable acids, and include for example those formed with inorganic acids e.g. hydrochloric, sulphuric or phosphoric acids and organic acids e.g. succinic, maleic, acetic or fumaric acid. Other non-pharmaceutically acceptable salts e.g. oxalates may be used for example in the isolation of compounds of Formula 1 for laboratory use, or for subsequent conversion to a pharmaceutically acceptable acid addition salt. Also included within the scope of the invention are base addition salts (such as sodium, potassium and ammonium salts), solvates and hydrates of compounds of the invention.

The conversion of a given compound to a desired compound salt is achieved by applying standard techniques, well known to one skilled in the art.

The present compounds are useful as pharmaceuticals for the treatment of various conditions in which the use of a glycine transport inhibitor is indicated.

Preferred compounds are those useful as pharmaceuticals for the treatment of medical conditions for which GlyT-2-mediated inhibition of glycine transport is needed, such as the treatment of pain or the treatment of diseases or conditions associated with increased muscle contraction, for example spasticity and myoclonus. Spasticity that can be treated via modulation of glycine transporters is that associated with epilepsy, stroke, head trauma, multiple sclerosis, spinal cord injury, dystonia, and other conditions of illness and injury of the nervous system. By GlyT-2 we mean those glycine transporters found predominantly in the brain stem and spinal cord and the distribution of which corresponds closely to that of strychnine-sensitive glycine receptors (Liu et al. J. Biological Chemistry, 268, 1993:22802-22808; Jursky and Nelson, J. Neurochemistry, 64, 1995:1026-1033) and as described in U.S. Pat. No. 5,700,013.

For use in medicine, the compounds of the present invention can be administered in a standard pharmaceutical composition. The present invention therefore provides, in a further aspect, pharmaceutical compositions comprising a pharmaceutically acceptable carrier and a Formula 1 compound or a pharmaceutically acceptable salt, solvate or hydrate thereof, in an amount effective to treat the target indication.

The compounds of the invention are, for instance, administered orally, sublingually, rectally, nasally, vaginally, topically (including the use of a patch or other transdermal delivery device), by pulmonary route by use of an aerosol, or parenterally, including, for example, intramuscularly, subcutaneously, intraperitoneally, intraarterially, intravenously or intrathecally. Administration can be by means of a pump for periodic or continuous delivery. The compounds of the invention are administered alone, or are combined with a pharmaceutically-acceptable carrier or excipient according to standard pharmaceutical practice. For the oral mode of administration, the compounds of the invention are used in the form of tablets, capsules, lozenges, chewing gum, troches, powders, syrups, elixirs, aqueous solutions and suspensions, and the like. In the case of tablets, carriers that are used include lactose, sodium citrate and salts of phosphoric acid. Various disintegrants such as starch, and lubricating agents such as magnesium stearate and talc, are commonly used in tablets. For oral administration in capsule form, useful diluents are lactose and high molecular weight polyethylene glycols. If desired, certain sweetening and/or flavoring agents are added. For parenteral administration, sterile solutions of the compounds of the invention are usually prepared, and the pHs of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzylchromium chloride, and the usual quantities of diluents and/or carriers. For pulmonary administration, diluents and/or carriers will be selected to be appropriate to allow the formation of an aerosol. Suppository forms of the compounds of the invention are useful for vaginal, urethral and rectal administrations. Such suppositories will generally be constructed of a mixture of substances that is solid at room temperature but melts at body temperature. The substances commonly used to create such vehicles include theobroma oil, glycerinated gelatin, hydrogenated vegetable oils, mixtures of polyethylene glycols of various molecular weight and fatty acid esters of polyethylene glycol. See, Remington's Pharmaceutical Sciences, 16th Ed., Mack Publishing, Easton, Pa., 1980, pp. 1530-1533 for further discussion of suppository dosage forms and other dosage forms. Analogous gels or creams can be used for vaginal, urethral and rectal administrations.

Numerous administration vehicles will be apparent to those of ordinary skill in the art, including without limitation slow release formulations, liposomal formulations and polymeric matrices.

Examples of pharmaceutically acceptable acid addition salts for use in the present invention include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic and arylsulphonic acids, for example.

Examples of pharmaceutically acceptable base addition salts for use in the present invention include those derived from non-toxic metals such as sodium or potassium, ammonium salts and organoamino salts such as triethylamine salts. Numerous appropriate such salts will be known to those of ordinary skill.

The physician or other health care professional can select the appropriate dose and treatment regimen based on the subject's weight, age, and physical condition. Dosages will generally be selected to maintain a serum level of compounds of the invention between about 0.01 μg/cc and about 1000 μg/cc, preferably between about 0.1 μg/cc and about 100 μg/cc. For parenteral administration, an alternative measure of preferred amount is from about 0.001 mg/kg to about 10 mg/kg (alternatively, from about 0.01 mg/kg to about 10 mg/kg), more preferably from about 0.01 mg/kg to about 1 mg/kg (from about 0.1 mg/kg to about 1 mg/kg), will be administered. For oral administrations, an alternative measure of preferred administration amount is from about 0.001 mg/kg to about 10 mg/kg (from about 0.1 mg/kg to about 10 mg/kg), more preferably from about 0.01 mg/kg to about 1 mg/kg (from about 0.1 mg/kg to about 1 mg/kg). For administrations in suppository form, an alternative measure of preferred administration amount is from about 0.1 mg/kg to about 10 mg/kg, more preferably from about 0.1 mg/kg to about 1 mg/kg.

For use in assaying for activity in inhibiting glycine transport, eukaryotic cells, preferably QT-6 cells derived from quail fibroblasts, have been transfected to express one of the four known variants of human GlyT-1, namely GlyT-1a, GlyT-1b, GlyT-1c or GlyT-1d, or human GlyT-2. The sequences of these GlyT-1 transporters are described in Kim et al., Molec. Pharm. 45: 608-617, 1994, excepting that the sequence encoding the extreme N-terminal of GlyT-1a was merely inferred from the corresponding rat-derived sequence. This N-terminal protein-encoding sequence has now been confirmed to correspond to that inferred by Kim et al. The sequence of the human GlyT-2 is described by Albert et al., U.S. Pat. No. 5,919,653, issued Jul. 6, 1999, which is incorporated herein by reference in its entirety. Suitable expression vectors include pRc/CMV (Invitrogen), Zap Express Vector (Stratagene Cloning Systems, LaJolla, Calif.; hereinafter “Stratagene”), pBk/CMV or pBk-RSV vectors (Stratagene), Bluescript II SK+/−Phagemid Vectors (Stratagene), LacSwitch (Stratagene), PMAM and PMAM neo (Clontech), among others. A suitable expression vector is capable of fostering expression of the included GlyT DNA in a suitable host cell, preferably a non-mammalian host cell, which can be eukaryotic, fungal, or prokaryotic. Such preferred host cells include amphibian, avian, fungal, insect, and reptilian cells.

Compounds of the present invention can be prepared by the method shown in Scheme I. In Scheme 1 the starting material is the amino acid A, the amino acid A is Boc protected at the Nitrogen to give the intermediate B. One method for carrying out the Boc protection is shown as I in Scheme 1. The Boc protected amino acid can then be reacted with an aniline as in method II to give the amide intermediate C. The Boc group is then removed to give the free amine D, which can then be reacted with an isothiocyanate to give the thiourea product E.

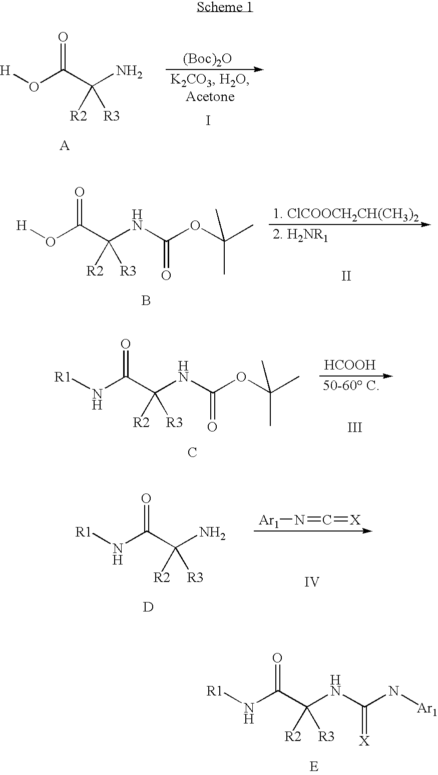

### EXAMPLES

General Procedures

I—Conversion of Amino Acid A to Boc Protected Product B

To a round bottom flask was added the amino acid (1 eq.), Et₃N (5 eq.) 1M NaOH (1 eq.) and CH₃CN. The clear solution was cooled to 0° C. and to it was added (Boc)₂O. The reaction was warmed to room temperature and stirred for four hours, during which time a white precipitate formed. The reaction mixture was concentrated and the residue was dissolved in EtOAc:water (1:1). The organic phase was washed with water and the aqueous phases were combined and treated with 10% HCl and then were extracted with EtOAc three times. The combined organic phase was washed successively with water and brine, then dried over MgSO₄, filtered and concentrated to yield the title compound.

II—Formation of Amide Intermediate C from Boc Protected Amino Acid, B, and a Primary Amine.

To a flame dried round bottom flask was added Boc protected-amino acid and CH₂Cl₂ (5 mL). The clear solution was cooled to 0° C. and the primary amine (1 eq.) was added followed by diisopropylethylamine (2 eq.) and N,N-bis(2-oxo-3-oxazolidinyl)phosphonic chloride (1 eq.). The white suspension was allowed to stir at 0° C. under Argon for two hours. The workup included pouring the clear reaction mixture into Ether:water (3:2). The organic layer was separated and was successively washed with 1N NaHSO₄, water, sat. NaHCO₃ and brine. It was dried over MgSO₄, filtered and concentrated to yield the title compound.

III— Boc-Deprotection of Amide C to Give Intermediate D

The intermediate amide C and formic acid (neat) were added to a sealed vial. The vial was heated at 60° C. in an oil bath for forty minutes. After cooling, the reaction mixture was concentrated and the residue was purified by an SPE tube using mixtures of CH₂Cl₂:MeOH as the eluent. A concentration gradient was used starting with 98:2 (CH₂Cl₂:MeOH) followed by (95:5), (94:6), (93:7), (92:8), (90:10), (85:15), (80:20) and finally 100% MeOH to yield the title compound.

IV—Formation of Final Product E from D and an Isothiocyanate.

The amine D (1 eq.), and the desired isothiocyanate (1.2 eq.), triethylamine (1 drop) and acetone (2 mL) were added to a sealed vial. The reaction was heated to 50° C. and was left stirring for four hours. The mixture was concentrated and the crude product was purified by an SPE tube using Hexanes:Ethyl Acetate (90:10), (80:20), (70:30), (60:40), (50:50) and finally (40:60) as the eluent to yield the title compound.

The compounds of examples 1 through 57 were made from the indicated starting materials by the general synthetic procedures described above unless otherwise noted.

### EXPERIMENTAL

In the experimental section each example describes the formation of a series of intermediate compounds (A, B, C, and D) and the formation of one or more final products E, by the reaction of intermediate D with one or more reagents. When more than one final product E is made from one intermediate D the final products are given differentiating numbers after the decimal point (for example E1.1 and E1.2). While the general scheme shows four steps for making the final product some of the desired intermediates were found to be commercially available. In cases where an intermediate is commercially available the example starts with that intermediate (and not with A). For example, experiment 1 begins with commercially available intermediate C1 which is converted to D1 which is then reacted with two different reagents to produce two different final products E1.1 and E1.2 Finally, some of the compounds were produced as single enantiomers while others were produced as a mixture. A “*” in the numbering system indicates the (R) enantionmer and “**” in the numbering system indicates the (S) enantiomer.

### Example 1

C1 Tert-butyl[1-(2-methylphenylcarbamoyl)-1-methyl-methyl]-carbamate

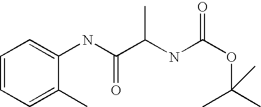

To a 50 mL flame dried round bottom flask was added Boc-dl-alanine and CH₂Cl₂ (5 mL). The clear solution was cooled to 0° C. and o-toluidine (0.12 mL, 1.16 mmol) was added followed by diisopropylethylamine (0.41 mL, 2.33 mmol) and N,N-bis(2-oxo-3-oxazolidinyl)phosphonic chloride (0.30 g, 1.16 mmol). The white suspension was allowed to stir at 0° C. under Argon for two hours. The workup included pouring the clear reaction mixture into ether (30 mL) and water (20 mL). The organic layer was separated and was successively washed with 1N NaHSO₄ (20 mL), water (20 mL), sat. NaHCO₃ (20 mL) and brine (20 mL). It was dried over MgSO₄, filtered and concentrated to yield the title compound as a white solid (151.0 mg, 51%).

D1 2-Amino-N-(2-methyl phenyl)-2-methyl acetamide formic acid salt

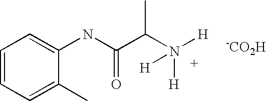

Tert-butyl[1-(2-methylphenylcarbamoyl)-1-methyl-methyl]-carbamate (70.0 mg, 0.25 mmol) and formic acid (2 mL) were added to a screw cap vial. The vial was heated at 60° C. in an oil bath for forty minutes. After cooling, the reaction mixture was concentrated and the residue was purified by an SPE tube using first CH₂Cl₂:MeOH 98:2 followed by 95:5, 94:6, 93:7, 92:8, 90:10, 85:15, 80:20 and finally 100% MeOH to yield the title compound as a white solid (32.3 mg, 72%).

E1.1 N-(2-methylphenyl)-2-[3-(4-phenylacetate)-thioureido]-2-methyl acetamide

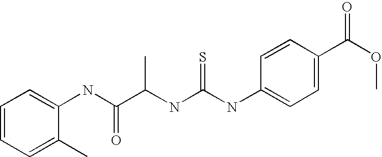

2-Amino-N-(4-methyl phenyl)-2-methyl acetamide formic acid salt (27.7 mg, 0.16 mmol), 4-methoxycarbonyl phenyl isothiocyanate (36.0 mg, 0.19 mmol), triethylamine (1 drop) and acetone (2 mL) were added to a screw cap vial. The reaction was heated to 50° C. and was left stirring for four hours. The crude product was purified by an SPE tube using Hexanes:Ethyl Acetate (90:10), (80:20), (70:30), (60:40), (50:50) and finally (40:60) as the eluent to yield the title compound as a white solid (7.1 mg, 12%).

E1.2 N-(2-methylphenyl)-2-[3-(2-nitrophenyl)-thioureido]-2-methyl acetamide

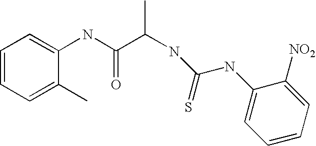

N-(2-methylphenyl)-2-[3-(2-nitrophenyl)-thioureido]-2-methyl acetamide was isolated as a yellow solid (27.5 mg, 59%) from 2-Amino-N-(2-methyl phenyl)-2-methyl acetamide formic acid salt (28.8 mg, 0.13 mmol), 2-nitrophenylisothiocyanate (27.8 mg, 0.15 mmol) and triethylamine (0.02 mL, 0.17 mmol).

### Example 2

C2 Tert-butyl[1-(2-methylphenylcarbamoyl)-1-tert-butyl-methyl]-carbamate

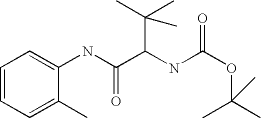

Tert-butyl[1-(2-methylphenylcarbamoyl)-1-tert-butyl-methyl]-carbamate was isolated as a white solid (63.0 mg, 14%) from [tert-butoxycarbonyl)amino](tert butyl)acetic acid (319.3 mg, 1.38 mmol) and o-toluidine (0.16 mL, 1.52 mmol), BOP-Cl (386.6 mg, 1.52 mmol) and diisopropylethylamine (0.53 mL, 3.04 mmol).

D2 2-Amino-N-(2-methylphenyl)-2-tert-butyl acetamide formic acid salt

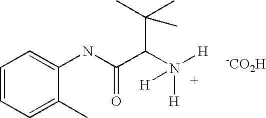

2-Amino-N-(2-methylphenyl)-2-tert-butyl acetamide formic acid salt was isolated as a white solid (51.4 mg, 96%) from Tert-butyl[1-(2-methylphenylcarbamoyl)-1-tert-butyl-methyl]-carbamate (63.0 mg, 0.20 mmol) and formic acid (2 mL).

E2.1 N-(2-methylphenyl)-2-[3-(4-phenylacetate)-thioureido]-2-tert butyl acetamide

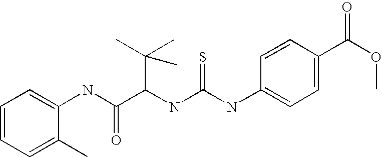

N-(2-methylphenyl)-2-[3-(4-phenylacetate)-thioureido]-2-tert butyl acetamide was isolated as a white solid (19.1 mg, 42%) from 2-Amino-N-(2-methylphenyl)-2-tert-butyl acetamide formic acid salt (25.0 mg, 0.11 mmol), 4-methoxycarbonyl phenylisothiocyanate (26.3 mg, 0.14 mmol) and triethylamine (1 drop).

### Example 3

C3 Tert-butyl[1-(2-methylphenylcarbamoyl)-1-isopropyl-methyl]-carbamate

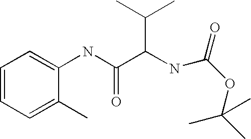

Tert-butyl[1-(2-methylphenylcarbamoyl)-1-isopropyl-methyl]-carbamate was isolated as a white solid (81.0 mg, 19%) from [tert-butoxycarbonyl)amino](isopropyl)methyl acetic acid (300 mg, 1.38 mmol) and o-toluidine (0.16 mL, 1.52 mmol), diisopropylethylamine (0.53 mL, 3.04 mmol) and BOP-Cl (386.6 mg, 1.52 mmol).

D3 2-Amino-N-(2-methylphenyl)-2-isopropyl acetamide formic acid salt

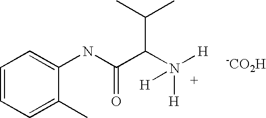

2-Amino-N-(2-methylphenyl)-2-isopropyl acetamide formic acid salt was isolated as a white solid (53.3 mg, 81%) from Tert-butyl[1-(2-methylphenylcarbamoyl)-1-isopropyl-methyl]-carbamate (81.0 mg, 0.26 mmol).

E3.1 N-(2-methylphenyl)-2-[3-(4-methoxycarbonyl)-thioureido]-2-isopropyl acetamide

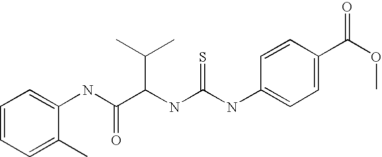

N-(2-methylphenyl)-2-[3-(4-methoxycarbonyl)-thioureido]-2-isopropyl acetamide was isolated as a clear oil (6.4 mg, 13%) from 2-Amino-N-(2-methylphenyl)-2-isopropyl acetamide formic acid salt (25.0 mg, 0.12 mmol), 4-methoxycarbonyl phenyl isothiocyanate (28.1 mg, 0.15 mmol) and triethylamine (0.02 mL, 0.15 mmol).

E3.2 N-(2-methylphenyl)-2-[3-(2-nitrophenyl)-thioureido]-2-isopropyl acetamide

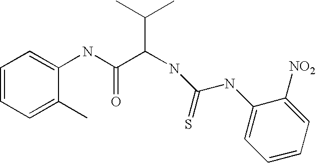

N-(2-methylphenyl)-2-[3-(2-nitrophenyl)-thioureido]-2-isopropyl acetamide was isolated as a yellow solid (30.0 mg, 65%) from 2-Amino-N-(2-methylphenyl)-2-isopropyl acetamide formic acid salt (31.4 mg, 0.12 mmol), 2-nitrophenylisothiocyanate (26.9 mg, 0.15 mmol) and triethylamine (0.02 mL, 0.16 mmol).

C3* (R)-Tert-butyl[1-(2-methylphenylcarbamoyl)-1-isopropyl-methyl]-carbamate

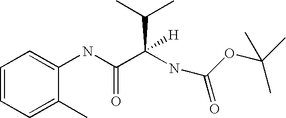

Boc-D-val-OH (163.8 mg, 0.75 mmol), o-toluidine (0.09 mL, 0.83 mmol), 1-methylimidazole (0.12 mL, 1.51 mmol) and DMF (2 mL) were added to a screw cap vial. The solution was cooled to 0° C. and to it was added diethylcyanophosphonate (0.19 mL, 1.13 mmol) dropwise. The resulting solution was stirred at room temperature for 72 hours. The reaction was diluted with ethyl acetate and the organic phase was washed successively with water, sodium hydrogensulphate (20%), water, NaHCO₃ (sat.), water and brine. It was then dried over MgSO₄, filtered and concentrated. The crude product was purified by an SPE tube (Hexanes:ethyl acetate 98:2 to 70:30) to yield the title compound as an off-white solid (81.0 mg, 35%)

D3* (R)-2-Amino-N-(2-methylphenyl)-2-isopropyl acetamide formic acid salt

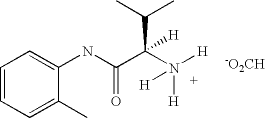

(R)-2-Amino-N-(2-methylphenyl)-2-isopropyl acetamide (formic acid salt) was isolated as a light yellow solid (32.4 mg, 58%) from (R)-Tert-butyl[1-(2-methylphenylcarbamoyl)-1-isopropyl-methyl]-carbamate (67.6 mg, 0.221 mmol) and Formic acid (96%) (2 mL).

E3.1* (R)-N-(2-methylphenyl)-2-[3-(4-methoxycarbonylphenyl)-thioureido]-2-isopropyl acetamide

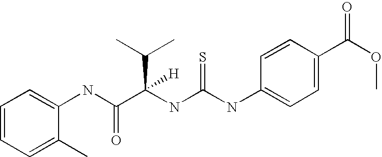

(R)-N-(2-methylphenyl)-2-[3-(4-methoxycarbonylphenyl)-thioureido]-2 isopropyl acetamide was isolated as an off-white solid (13.0 mg, 54%) from (R)-2-Amino-N-(2-methylphenyl)-2-isopropyl acetamide formic acid salt (14.3 mg, 0.06 mmol), 4-methoxy carbonylphenylisothiocyanate (13.1 mg, 0.07 mmol) and triethylamine (0.02 mL, 0.11 mmol).

E3.2* (R)-N-(2-methylphenyl)-2-[3-(2-nitrophenyl)-thioureido]-2-isopropyl acetamide

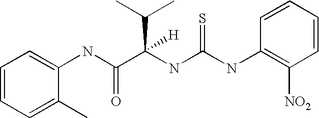

(R)-N-(2-methylphenyl)-2-[3-(2-nitrophenyl)-thioureido]-2-isopropyl acetamide was isolated as a yellow solid (14.7 mg, 54%) from (R)-2-Amino-N-(2-methylphenyl)-2-isopropyl acetamide formic acid salt (17.0 mg, 0.07 mmol), 2-nitrophenylisothiocyanate (14.6 mg, 0.08 mmol) and triethylamine (0.02 mL, 0.13 mmol).

C3** (S)-Tert-butyl[1-(2-methylphenylcarbamoyl)-1-isopropyl-methyl]-carbamate

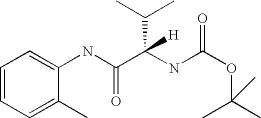

(S)-Tert-butyl[1-(2-methylphenylcarbamoyl)-1-isopropyl-methyl]-carbamate was isolated (129.2 mg, 56%) from Boc-L-val-OH (163.8 mg, 0.75 mmol), o-toluidine (0.09 mL, 0.83 mmol), diethylcyanophosphonate (90%) (0.19 mL, 1.13 mmol) and 1-methylimidazole (0.12 mL, 1.51 mmol).

D3** (S)-2-Amino-N-(2-methylphenyl)-2-isopropyl acetamide formic acid salt

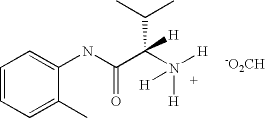

(S)-2-Amino-N-(2-methylphenyl)-2-isopropyl acetamide (formic acid salt) was isolated as a yellow solid (62.5 mg, 59%) from (S)-Tert-butyl[1-(2-methylphenylcarbamoyl)-1-isopropyl-methyl]-carbamate (129.2 mg, 0.422 mmol).

E3.1** (S)-N-(2-methylphenyl)-2-[3-(4-methoxycarbonylphenyl)-thioureido]-2-isopropyl acetamide

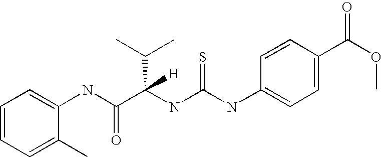

(S)-N-(2-methylphenyl)-2-[3-(4-methoxycarbonylphenyl)-thioureido]-2-isopropyl acetamide was isolated as an off-white solid (34.1 mg, 71%) from (S)-2-Amino-N-(2-methylphenyl)-2-isopropyl acetamide formic acid salt (29.3 mg, 0.12 mmol), 4-methoxycarbonyl phenyl isothiocyanate (26.9 mg, 0.14 mmol) and triethylamine (0.03 mL, 0.23 mmol).

E3.2** (S)-N-(2-methylphenyl)-2-[3-(2-nitrophenyl)-thioureido]-2-isopropyl acetamide

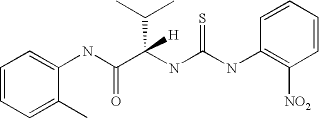

(S)-N-(2-methylphenyl)-2-[3-(2-nitrophenyl)-thioureido]-2-isopropyl acetamide was isolated as a yellow solid (31.4 mg, 68%) from (S)-2-Amino-N-(2-methylphenyl)-2-isopropyl acetamide formic acid salt (30.8 mg, 0.12 mmol), 2-nitrophenylisothiocyanate (26.4 mg, 0.15 mmol) and triethylamine 0.03 mL, 0.24 mmol).

### Example 4

B4 N-Tert-butoxycarbonyl DL-phenyl glycine

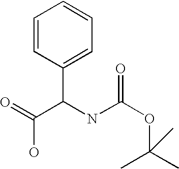

N-Tert-butoxycarbonyl DL-phenyl glycine was isolated as a white solid (7.61 g, 92%) from DL-2-phenylglycine (5.0 g, 33.1 mmol), 1N NaOH (132.4 mL, 132.4 mmol) and (BOC)₂O (19.0 mL, 82.7 mmol).

C4 tert-butyl[1-(2-methylphenylcarbamoyl)-1-phenyl-methyl]-carbamate

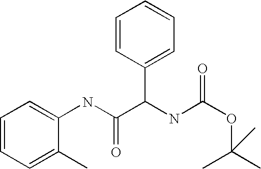

N-Tert-butoxycarbonyl DL-phenyl glycine (2.50 g, 9.95 mmol) was dissolved in dry THF (27 mL) in a flame dried flask under Argon. The solution was cooled to −50° C. and N-methylmorpholine (1.11 g, 10.95 mmol) and isobutylchloroformate (1.50 g, 10.95 mmol) were added. The reaction was allowed to stir at this temperature for 2.5 hours. N-methylmorpholine (1.20 g, 11.94 mmol) was added to o-toluidine in THF (3 mL). This solution was added to the reaction and the reaction was stirred overnight at which time it warmed to room temperature. The THF was then evaporated and CH₂Cl₂ (250 mL) was added. The solution was poured into a separatory funnel and NaHCO₃ (sat.) was added. The organic phase was isolated and washed with NaHCO₃ (sat), water and brine. The organic layer was then dried over Na₂SO₄, filtered and concentrated to yield a white solid (3.32 g, 98%).

D4 2-Amino-N-(2-methylphenyl)-2-phenylacetamide

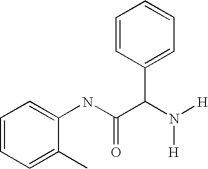

tert-butyl[1-(2-methylphenylcarbamoyl)-1-phenyl-methyl]-carbamate (2.00 g, 5.88 mmol) was dissolved in formic acid (20 mL) and the solution was allowed to stir for two hours at 50° C. under Argon. The flask was cooled to room temperature and the formic acid was evaporated. The resultant oil was dissolved in CH₂Cl₂ and poured into a separatory funnel. 1N NaOH was added and the product was extracted with CH₂Cl₂ three times. The combined organic layers were washed with water and brine, dried over NaSO₄, filtered and concentrated. The resultant oil was dissolved in a small amount of EtOAc and Hexane was added slowly. The solution became cloudy and a white solid precipitated (780.0 mg, 56%). The mother liquor was removed by pipette and the solid was washed with Hexane three times and was dried under vacuum.

E4.1 N-(2-methylphenyl)-2-[3-(4-methoxycarbonylphenyl)-thioureido]-2-phenyl acetamide

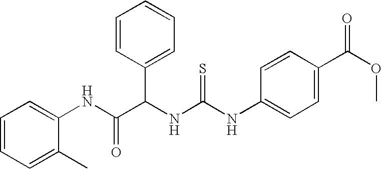

To a screw cap vial equipped with a stir bar was added 4-methoxycarbonyl phenylisothiocyanate (46.4 mg, 0.24 mmol), 2-Amino-N-(2-methylphenyl)-2-phenylacetamide formic acid salt (48.1 mg, 0.20 mmol), triethylamine (0.04 mL, 0.26 mmol) and CH₂Cl₂ (2 mL). The reaction vial was capped and the reaction was allowed to stir at room temperature for two hours. The reaction was not complete and was further heated to 50° C. for two hours. The reaction was then cooled and concentrated. The residue was diluted with Ethyl acetate. The organic layer was washed with water, then brine, dried over Na₂SO₄, filtered and concentrated. The crude product was purified via SPE tube (Hexanes:Et₂O 98:2 to Et₂O 100%) to yield the title compound as a yellow solid (55.0 mg, 64%).

E4.2 N-(2-methylphenyl)-2-[3-(2-nitrophenyl)-thioureido]-2-phenyl acetamide

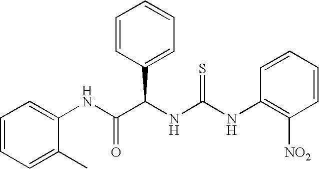

N-(2-methylphenyl)-2-[3-(2-nitrophenyl)-thioureido]-2-phenyl acetamide was isolated as a yellow solid (21.9 mg, 40%) from 2-Amino-N-(2-methylphenyl)-2-phenylacetamide formic acid salt (37.1 mg, 0.13 mmol), 2-nitrophenylisothiocyanate (28.0 mg, 0.16 mmol) and triethylamine (0.02 mL, 0.17 mmol).

E4.3 N-(2-methylphenyl)-2-[3-(4-ethoxy-2-nitrophenyl)-thioureido]-2-phenyl acetamide

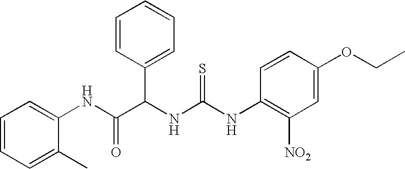

N-(2-methylphenyl)-2-[3-(4-ethoxy-2-nitrophenyl)-thioureido]-2-phenyl acetamide was isolated as a yellow solid (458.2 mg, 80%) from 2-Amino-N-(2-methylphenyl)-2-phenylacetamide (299.1 mg, 1.24 mmol) and 4-ethoxy-2-nitrophenylisothiocyanate (334.9 mg, 1.49 mmol).

E4.4 N-(2-methylphenyl)-2-[3-(2-nitro-4-methoxyphenyl)-thioureido]-2-phenyl acetamide

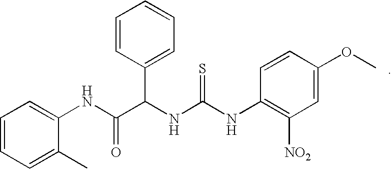

2-Amino-N-(4-methylphenyl)-2-phenylacetamide (23.7 mg, 0.099 mmol) was dissolved in CH₂Cl₂ (2.0 mL) in a test tube and 4-methoxy-2-nitrophenyl isothiocyanate (31.3 mg, 0.149 mmol) was added. The test tube was sealed and the reaction was allowed to stir at 50° C. for one hour. The reaction was cooled and concentrated and the residue was washed with 10% EtOAc in Hexanes. A yellow solid precipitated and was washed three times with 20% EtOAc in Hexanes. The compound was purified by SPE tube using first 10% EtOAc in Hexanes followed by 20% EtOAc in Hexanes followed by pure EtOAc as the eluant. The product was isolated as a bright yellow solid (17.6 mg, 39%).

E4.5 N-(2-methylphenyl)-2-[3-(2-trifluoromethylphenyl)-thioureido]-2-phenyl acetamide

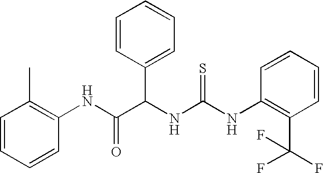

N-(2-methylphenyl)-2-[3-(2-trifluoromethylphenyl)-thioureido]-2-phenyl acetamide (ALX4097XX) (29.9 mg, 56%) was isolated as a white solid from 2-Amino-N-(4-methylphenyl)-2-phenylacetamide (28.8 mg, 0.120 mmol) and 2-trifluoromethylphenyl isothiocyanate (36.6 mg, 0.180 mmol).

E4.6 N-(2-methylphenyl)-2-[3-(1-naphthyl)-thioureido]-2-phenyl acetamide

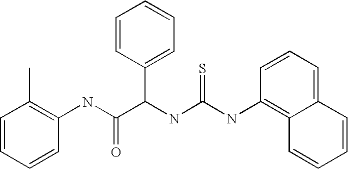

N-(2-methylphenyl)-2-[3-(2-naphthyl)-thioureido]-2-phenyl acetamide (22.1 mg, 43%) was isolated as a white solid from 2-Amino-N-(4-methylphenyl)-2-phenylacetamide (29.0 mg, 0.121 mmol) and 1-naphthyl isothiocyanate (33.7 mg, 0.182 mmol).

E4.7 N-(2-methylphenyl)-2-[3-(4-N,N-dimethylaminoethoxy-2-nitrophenyl)-thioureido]-2-phenyl acetamide

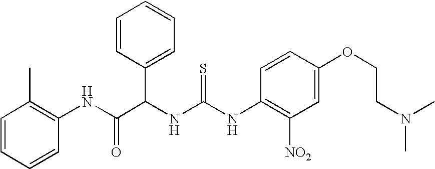

To a screw capped vial containing 4-[2-N,N-dimethylaminoethoxy]-2-nitrophenyl acetamide (80.0 mg, 0.30 mmol) was added 20% KOH (aq) (3 mL). The mixture was heated to reflux for two hours producing an orange solution. This mixture was cooled and extracted with CH₂Cl₂ three times and the combined organic layers were dried with anhydrous Na₂SO₄ and concentrated to yield the crude product. EtOAc (2 mL) was added followed by thiophosgene (0.05 mL, 0.66 mmol). The mixture was stirred at 75° C. for one hour after which the solvent was removed and the crude product was dissolved in CH₂Cl₂ (2 mL) and 2-amino-N-(2-methylphenyl)-2-phenylacetamide (60.0 mg, 0.25 mmol) was added and the mixture was stirred at room temperature overnight. The solvent was evaporated and the crude residue was purified by an SPE tube (5% MeOH/NH₃ in CH₂Cl₂) to yield the title compound as a yellow solid (25.0 mg, 16%)

E4.8 N-(2-methylphenyl)-2-[3-phenylthioureido]-2-phenyl acetamide

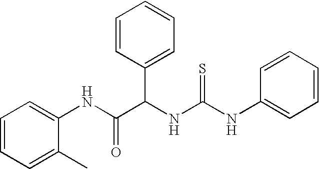

N-(2-methylphenyl)-2-[3-phenylthioureido]-2-phenyl acetamide was isolated as a white solid, (20.6 mg, 69%) from phenylisothiocyanate (17 mg, 0.13 mmol) and N-(2-methylphenyl)phenylglycinamide (20 mg, 0.08 mmol).

C4* (R)-tert-butyl[1-(2-methylphenylcarbamoyl)-1-phenyl-methyl]-carbamate

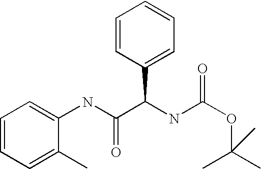

(R)-tert-butyl[1-(2-methylphenylcarbamoyl)-1-phenyl-methyl]-carbamate was isolated as an off-white solid (641.4 mg, 47%) from Boc-D-PHG-OH (1.0 g, 3.98 mmol), o-toluidine (0.47 mL, 4.38 mmol), 1-methylimidazole (0.63 mL, 7.96 mmol) and diethylcyanophosphonate (90%) (1.01 mL, 5.97 mmol)

D4* (R)-2-Amino-N-(2-methylphenyl)-2-phenylacetamide formic acid salt

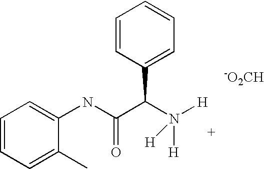

(R)-2-Amino-N-(2-methylphenyl)-2-phenylacetamide was isolated as an off-white solid (452.5 mg, 84%) from (R)-tert-butyl[1-(2-methylphenylcarbamoyl)-1-phenyl-methyl]-carbamate (641.4, 1.88 mmol).

E4.1* (R)-N-(2-methylphenyl)-2-[3-(2-nitrophenyl)-thioureido]-2-phenyl acetamide

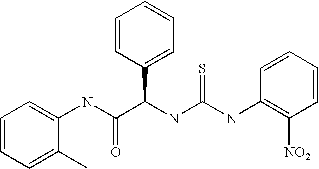

(R)-N-(2-methylphenyl)-2-[3-(2-nitrophenyl)-thioureido]-2-phenyl acetamide was isolated as a dark yellow solid (237.7 mg, 81%) from (R)-2-Amino-N-(4-methylphenyl)-2-phenylacetamide formic acid salt (200.0 mg, 0.70 mmol) and 2-nitrophenylisothiocyanate (151.0 mg, 0.84 mmol) and triethylamine (0.19 mL, 1.40 mmol).

E4.2* N-(2-methylphenyl)-2-[3-(4-methoxy-2-nitrophenyl)-thioureido]-2-phenyl acetamide

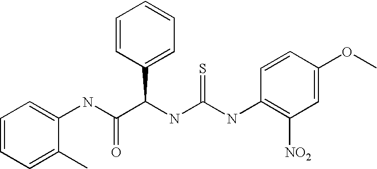

N-(2,6-dimethylphenyl)-2-[3-(4-methoxy-2-nitrophenyl)-thioureido]-2-phenyl acetamide was isolated as a yellow solid (R isomer 76%, S-isomer 24%) from (R)-2-Amino-N-(4-methylphenyl)-2-phenylacetamide (30.0 mg, 0.12 mmol) and 4-methoxy-2-nitrophenyliso thiocyanate (29.8 mg, 0.14 mmol).

### Example 5

B5 1-(Tert-butoxycarbonylamino)cyclopentanecarboxylic acid

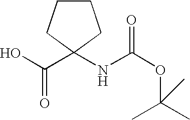

To a 250 mL round bottom flask was added cycloleucine (1.0 g, 7.74 mmol), Et₃N (5.4 mL, 38.7 mmol) 1M NaOH (7.74 mL, 7.74 mmol) and CH₃CN (10 mL). The clear solution was cooled down to 0° C. and to it was added (Boc)₂O. The reaction was warmed to room temperature and stirred for four hours, during which time a white precipitate formed. The reaction mixture was concentrated and the residue was dissolved in EtOAc:water (1:1) (100 mL). The organic phase was washed with water and the aqueous phases were combined and treated with 10% HCl and then were extracted with EtOAc three times. The combined organic phase was washed successively with water, brine, dried over MgSO₄, filtered and concentrated to yield the title compound as a white solid (1.29 g, 73%).

C5 1-1-tert-butyl-carbamoyl-1-[(N-(2-methyl phenyl)]-cyclopentane carboxamide

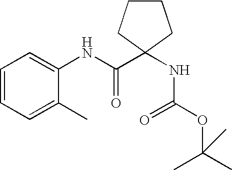

1-tert-butyl-carbomoyl-1-[N-(2-methylphenyl)]-cyclopentane carboxamide was isolated as a white solid (32.0 mg, 8%) from 1-(Tert-butoxycarbonylamino) cyclopentanecarboxylic acid (300 mg, 1.31 mmol) o-toluidine (0.15 mL, 1.44 mmol), diisopropylethylamine (0.50 mL, 2.88 mmol) and BOP-Cl (366.4 mg, 1.44 mmol).

D5 2-Amino-N-(2-methylphenyl)-2-cyclopentyl acetamide formic acid salt

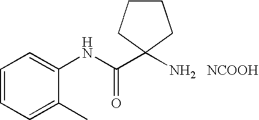

2-Amino-N-(2-methylphenyl)-2-cyclopentyl acetamide formic acid salt was isolated as a white solid (16.6 mg, 85%) from 1-tert-butyl-carbomoyl-1-[N-(2-methylphenyl)]-cyclopentane carboxamide (30.0 mg, 0.09 mmol).

E5.1 [1-(4-methoxycarbonylphenyl)-thioureido N-(2-methylphenyl)]-cyclopentane carboxamide

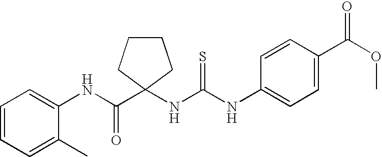

[1-(4-methoxycarbonylphenyl)-thioureido N-(2-methylphenyl)]-cyclopentane carboxamide was isolated as a white solid (19.5 mg, 79%) from 1-Amino-N-(2-methylphenyl)cyclopentane carboxamide formic acid salt (15.2 mg, 0.06 mmol), 4-methoxycarbonylphenylisothiocyanate (13.3 mg, 0.07 mmol) and triethylamine (0.01 mL, 0.07 mmol).

E5.2 [1-(2-nitrophenyl)-thioureido N-(2-methylphenyl)]-cyclopentane carboxamide

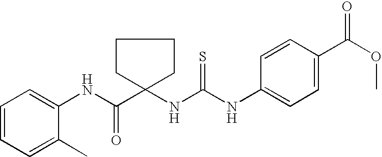

1-(2-nitrophenyl)-thioureido N-(2-methylphenyl)]-cyclopentane carboxamide was isolated as a yellow solid (5.1 mg, 32%) from 2-amino-N-(2-methylphenyl)-2-cyclopentyl acetamide formic acid salt (10.3 mg, 0.04 mmol), 2-nitrophenylisothiocyanate (8.4 mg, 0.05 mmol) and triethylamine (0.01 mL, 0.05 mmol).

### Example 6

B6 1-(Tert-butoxycarbonylamino) cyclohexanecarboxylic acid

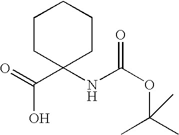

Tetramethylammonium hydroxide (1.27 g, 6.98 mmol) was added to 1-amino-1-cyclohexane carboxylic acid (1.0 g, 6.98 mmol) in CH₃CN (20 mL). The mixture was allowed to stir for 45 minutes at which time (Boc)₂O (3.05 g, 13.97 mmol) was added and the reaction was allowed to stir at room temperature for three hours. The solvent was then evaporated and Et₂O was added. The Et₂O layer was extracted with water twice. The combined water layers were then acidified with 10% HCl and EtOAc was added. The product was extracted with EtOAc three times. The combined EtOAc layers were washed with brine, dried over Na₂SO₄, filtered and concentrated to yield the title compound (630.0 mg, 37%) as a white solid.

C6 1-tert-butyl-carbamoyl-1-[N-(2-methylphenyl)]cyclohexane carboxamide

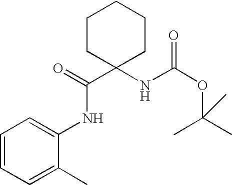

To a round bottom flask was added 1-(Tert-butoxycarbonylamino) cyclohexanecarboxylic acid (630.0 mg, 2.59 mmol), o-toluidine (0.31 g, 2.85 mmol) and 1-methylimidazole (0.43 g, 5.19 mmol) in DMF (15 mL) under Argon. Diethylcyanophosphonate was added dropwise while the flask was cooled in ice. The reaction was allowed to stir for three days during which time it warmed to room temperature. NaHCO₃ (sat) was added and a white precipitate formed. The reaction was poured into a separatory funnel and 1M NaHSO₄ was added and the precipitate dissolved. The product was extracted with EtOAc three times and the combined organic layers were washed with brine, dried over NaSO₄, filtered and concentrated. The crude product was purified by column chromatography (15% EtOAc in Hexanes) to yield the title compound (230 mg, 27%) as a white solid.

D6 1-Amino-N-(2-methylphenyl)cyclohexane carboxamide

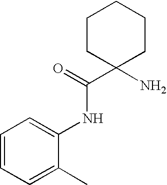

1-Amino-N-(2-methylphenyl)cyclohexane carboxamide (564.0 mg, 79%) was isolated as a white solid from 1-tert-butyl-carbamoyl-1-[N-(2-methylphenyl)] cyclohexane carboxamide (0.850 g, 2.56 mmol).

E6.1 [1-(4-methoxycarbonylphenyl)-thioureido N-(2-methylphenyl)]-cyclohexane carboxamide

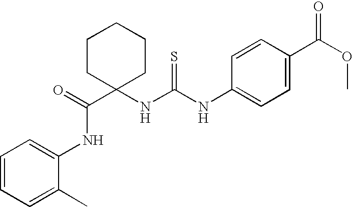

[1-(4-methoxycarbonylphenyl)-thioureido N-(2-methylphenyl)]-cyclohexane carboxamide was isolated as a white solid (3.0 mg, 35%) from 1-Amino-N-(2-methylphenyl)cyclohexane carboxamide formic acid salt (3.6 mg, 0.02 mmol), 4-methoxycarbonylphenylisothiocyanate (3.6 mg, 0.02 mmol) and triethylamine (0.01 mL, 0.02 mmol).

E6.2 [1-(2-nitrophenyl)-thioureido N-(2-methylphenyl)]-cyclohexane carboxamide

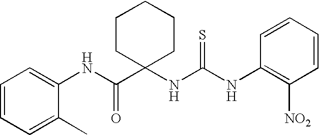

[1-(2-nitrophenyl)-thioureido N-(2-methylphenyl)]-cyclohexane carboxamide was isolated as a yellow solid (19.7 mg, 68%) from 1-Amino-N-(2-methylphenyl)cyclohexane carboxamide formic acid salt (18.4 mg, 0.07 mmol), 2-nitrophenylisothiocyanate (14.3 mg, 0.08 mmol) and triethylamine (0.01 mL, 0.09 mmol).

### Example 7

B7 2-[tert-butoxycarbonyl)amino]-2-(trifluoromethyl)acetic acid

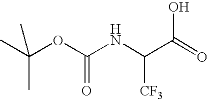

To a 50 mL round bottom flask was added DL-3,3,3-Trifluoro-2-alanine (419.8 mg, 2.93 mmol), tetramethylammonium hydroxide (1.10 g, 6.05 mmol) and acetonitrile (20 mL). The reaction was allowed to stir until the mixture turned clear (approx. 30 minutes). To the solution was added (Boc)₂O (1.35 mL, 5.87 mmol) and the clear solution was allowed to stir for 2.5 hours. The reaction was concentrated, and the residue was diluted with diethyl ether. The aqueous phase was extracted with diethyl ether and acidified with 10% HCl. The aqueous phase was extracted with ethyl acetate and the combined organic layers were washed with brine, dried over magnesium sulphate, filtered and concentrated to yield the title compound as a white solid (644.0 mg, 90%).

C7 Tert-butyl[1-(2-methylphenylcarbamoyl)-1-trifluoromethyl-methyl]-carbamate

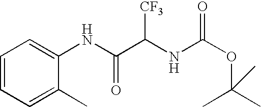

Tert-butyl[1-(2-methylphenylcarbamoyl)-1-trifluoromethyl-methyl]-carbamate was isolated (123.1 mg, 60%) from [tert-butoxycarbonylamino](trifluoromethyl)methyl acetic acid (183.3 mg, 0.75 mmol), 1-methylimidazole (0.12 mL, 1.51 mmol), o-toluidine (0.09 mL, 0.83 mmol) and diethylcyanophosphonate (0.19 mL, 1.13 mmol).

D7 2-Amino-N-(2-methylphenyl)-2-trifluoromethyl acetamide

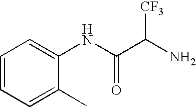

2-Amino-N-(2-methylphenyl)-2-trifluoromethyl acetamide was isolated as an off-white solid (17.5 mg, 17%) from Tert-butyl[1-(2-methylphenylcarbamoyl)-1-trifluoromethyl-methyl]-carbamate (123.1 mg, 0.447 mmol).

E7.1 N-(2-methylphenyl)-2-[3-(4-methoxycarbonylphenyl)-thioureido]-2-trifluoromethyl acetamide

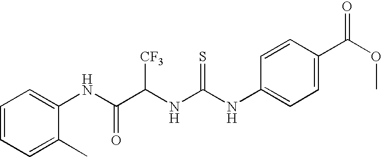

N-(2-methylphenyl)-2-[3-(4-methoxymethylphenyl)-thioureido]-2-trifluoromethyl acetamide was isolated as an off-white solid (7.5 mg, 59%) from 2-Amino-N-(2-methylphenyl)-2-trifluoromethyl acetamide (8.2 mg, 0.03 mmol), 4-methoxycarbonylphenylisothiocyanate (6.8 mg, 0.04 mmol) and triethylamine (0.01 mL, 0.06 mmol).

### Example 8

B8 1-[tert-butoxycarbonyl)amino]-1-(benzyl)acetic acid

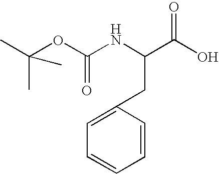

1-[tert-butoxycarbonyl)amino]-1-(benzyl)acetic acid was isolated as a white solid (1.46 g, 91%) from DL-phenylalanine (1.0 g, 6.05 mmol), tetramethylammonium hydroxide (1.01 g, 6.05 mmol) and (Boc)₂O (2.78 mL, 12.1 mmol).

C8 Tert-butyl[1-(2-methylphenylcarbamoyl)-1-benzyl-methyl]-carbamate

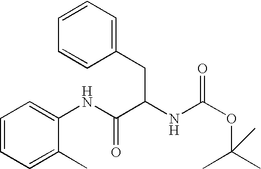

Tert-butyl[1-(2-methylphenylcarbamoyl)-1-benzyl-methyl]-carbamate was isolated (53.1 mg, 20%) from [tert-butoxycarbonyl)amino](benzyl)acetic acid (200.0 mg, 0.75 mmol), o-toluidine (0.09 mL, 0.83 mmol), 1-methylimidazole (0.12 mL, 1.51 mmol) and diethylcyanophosphonate (90%) (0.19 mL, 1.13 mmol).

D8 2-Amino-N-(2-methylphenyl)-2-benzyl acetamide formic acid salt

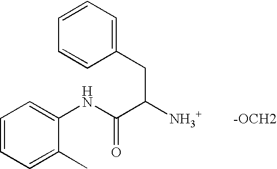

2-Amino-N-(2-methylphenyl)-2-benzyl acetamide (formic acid salt) was isolated as an off-white solid (40.9 mg, 91%) from Tert-butyl[1-(2-methylphenylcarbamoyl)-1-benzyl-methyl]-carbamate (53.1 mg, 0.150 mmol).

E8.1 N-(2-methylphenyl)-2-[3-(4-methoxycarbonylphenyl)-thioureido]-2-benzyl acetamide

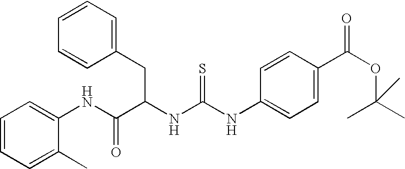

N-(2-methylphenyl)-2-[3-(4-methoxycarbonylphenyl)-thioureido]-2-benzyl acetamide was isolated as an off-white solid (19.7 mg, 88%) from 2-Amino-N-(2-methylphenyl)-2-benzyl acetamide formic acid salt (15.1 mg, 0.05 mmol), 4-methoxycarbonylisothiocyanate (11.7 mg, 0.06 mmol) and triethylamine (0.01 mL, 0.10 mmol).

E8.2 N-(2-methylphenyl)-2-[3-(2-nitrophenyl)-thioureido]-2-benzyl acetamide

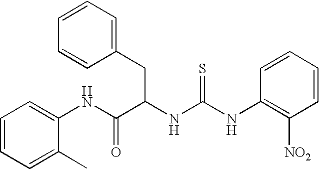

N-(2-methylphenyl)-2-[3-(2-nitrophenyl)-thioureido]-2-benzyl acetamide was isolated as a yellow solid (15.8 mg, 61%) from 2-Amino-N-(2-methylphenyl)-2-benzyl acetamide formic acid salt (18.9 mg, 0.06 mmol), 2-nitrophenylisothiocyanate (13.6 mg, 0.08 mmol) and triethylamine (0.02 mL, 0.13 mmol).

### Example 9

C9 tert-butyl[1-(2-methylphenylcarbamoyl)-1-dimethyl-methyl]-carbamate

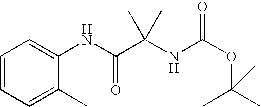

tert-butyl[1-(2-methylphenylcarbamoyl)-1-dimethyl-methyl]-carbamate was isolated as a white solid (206.0 mg, 48%) from Boc-AIB-OH (300.0 mg, 1.48 mmol), o-toluidine (0.17 mL, 1.62 mmol), 1-methylimidazole (0.23 mL, 2.95 mmol) and diethylcyanophosphonate (90%) (0.37 mL, 2.21 mmol).

D9 2-Amino-N-(2-methylphenyl)-2-dimethylacetamide formic acid salt

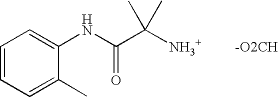

2-Amino-N-(2-methylphenyl)-2-dimethyl acetamide formic acid salt was isolated as a white solid (132.2 mg, 80%) from tert-butyl[1-(2-methylphenylcarbamoyl)-1-dimethyl-methyl]-carbamate (201.3 mg, 0.69 mmol).

E9.1 N-(2-methylphenyl)-2-[3-(2-nitrophenyl)-thioureido]-2-dimethyl acetamide

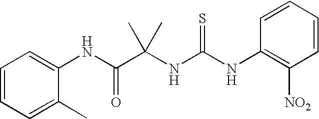

N-(2-methylphenyl)-2-[3-(2-nitrophenyl)-thioureido]-2-dimethyl acetamide was isolated as a yellow solid (15.0 mg, 37%) from 2-Amino-N-(2-methylphenyl)-2-dimethylacetamide formic acid salt (25.1 mg, 0.11 mmol), 2-nitrophenylisothiocyanate (22.7 mg, 0.13 mmol) and triethylamine (0.03 mL, 0.21 mmol).

E9.2 N-(2-methylphenyl)-2-[3-(4-methoxycarbonylphenyl)-thioureido]-2-dimethyl acetamide

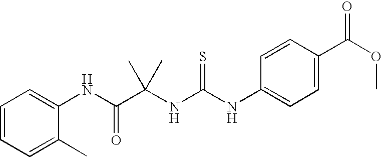

N-(2-methylphenyl)-2-[3-(4-methoxycarbonylphenyl)-thioureido]-2-dimethyl acetamide was isolated as a white solid (29.1 mg, 69%) from 2-Amino-N-(2-methylphenyl)-2-dimethylacetamide formic acid salt (25.1 mg, 0.11 mmol), 4-methoxycarbonyl isothiocyanate (26.4 mg, 0.14 mmol) and triethylamine 90.03 mL, 0.21 mmol).

### Example 10

C10 1-tert-butyl carbamoyl-1-[N-(2-methylphenyl)]-cyclopropane carboxamide

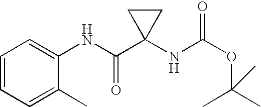

1-tert-butyl carbamoyl-1-[N-(2-methylphenyl)]-cyclopropane carboxamide was isolated as an off-white solid (532.9 mg, 74%) from 1-(N-tert-butoxycarbonyl-Amino)-cyclopropane carboxylic acid (500.0 mg, 2.48 mmol), o-toluidine (0.29 mL, 2.73 mmol), 1-methyl imidazole (0.40 mL, 4.97 mmol) and diethylcyanophosphonate (90%) (0.63 mL, 3.73 mmol).

D10 1-Amino-N-(2-methylphenyl)cyclopropane carboxamide

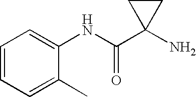

1-Amino-N-(2-methylphenyl)cyclopropane carboxamide was isolated as a white solid (140.6 mg, 41%) from 1-tert-butyl carbamoyl-1-[N-(2-methylphenyl)]-cyclopropane carboxamide (526.4 mg, 1.81 mmol).

E10.1 [1-(2-nitrophenyl)-thioureido-1-N-(2-methylphenyl)]-cyclopropane Carboxamide

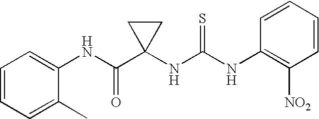

[1-(2-nitrophenyl)-thioureido N-(2-methylphenyl)]-cyclopropane carboxamide was isolated as a yellow solid (26.5 mg, 65%) from 1-Amino-N-(2-methylphenyl)cyclopropane carboxamide (20.0 mg, 0.11 mmol), 2-nitrophenylisothiocyanate (22.7 mg, 0.13 mmol) and triethylamine (0.03 mL, 0.21 mmol).

E10.2 [1-(4-methoxycarbonylphenyl)-thioureido N-(2-methylphenyl)]-cyclopropane carboxamide

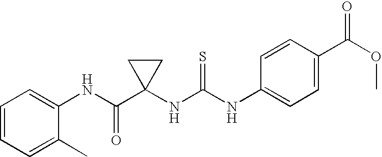

[1-(4-methoxycarbonylphenyl)-thioureido N-(2-methylphenyl)]-cyclopropane carboxamide was isolated as a white solid (14.6 mg, 35%) from 1-Amino-N-(2-methylphenyl)cyclopropane carboxamide (20.0 mg, 0.11 mmol), 4-methoxycarbonylisothiocyanate (26.4 mg, 0.14 mmol) and triethylamine (0.03 mL, 0.21 mmol).

### Example 11

C11 1-tert-butyl carbamoyl-1-[N-(2-methylphenyl)]-indanyl carboxamide

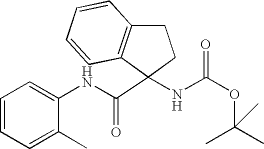

1-tert-butyl carbamoyl-1-[N-(2-methylphenyl)]-indanyl carboxamide was isolated as a pale yellow solid (28.0 mg, 43%) from (R,S)-tert-butoxycarbonyl-1-aminoindane-1-carboxylic acid (500 mg, 1.80 mmol), o-toluidine (0.21 mL, 1.98 mmol), 1-methylimidazole (0.29 mL, 3.61 mmol) and diethylcyanophosphonate (90%) (0.46 mL, 2.70 mmol).

D11 1-Amino-1-N-(2-methylphenyl)indanyl carboxamide formic acid salt

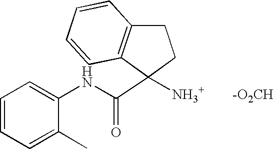

1-Amino-1-N-(2-methylphenyl)indanyl carboxamide was isolated as a light green foam (223.4 mg, 97%) from 1-tert-butyl carbamoyl-1-[N-(2-methylphenyl)]-indanyl carboxamide (272.3 mg, 0.74 mmol).

E11.1 [1-(2-nitrophenyl)-thioureido-1-N-(2-methylphenyl)]-indanyl carboxamide

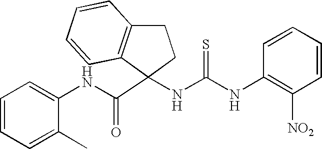

[1-(2-nitrophenyl)-thioureido-1-N-(2-methylphenyl)]-indanyl carboxamide was isolated as a yellow solid (26.9 mg, 55%) from 1-Amino-1-N-(2-methylphenyl)indanyl carboxamide formic acid salt (32.8 mg, 0.11 mmol), 2-nitrophenylisothiocyanate (22.7 mg, 0.13 mmol) and triethylamine (0.03 mL, 0.21 mmol).

E11.2 [1-(4-methoxycarbonylphenyl)-thioureido-1-N-(2-methylphenyl)]-indanyl carboxamide

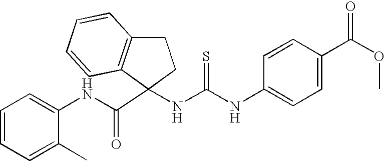

[1-(4-methoxycarbonylphenyl)-thioureido-1-N-(2-methyl phenyl)]-indanyl carboxamide was isolated as an off-white solid (21.1 mg, 57%) from 1-Amino-1-N-(2-methylphenyl)indanyl carboxamide formic acid salt (20.0 mg, 0.08 mmol) and 4-methoxycarbonylphenylisothiocyanate (18.9 mg, 0.10 mmol).

### Example 12

B12* (R)-2-[(tert-butoxycarbonyl)amino]-2-(cyclohexyl)acetic acid

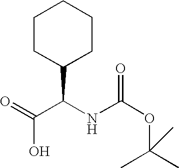

(R)-[(tert-butoxycarbonyl)amino](cyclohexyl)acetic acid (1.20 g, 73%) was isolated as a white foam from (R)-1-amino-1-cyclohexyl carboxylic acid (1.0 g, 6.36 mmol), tetramethylammonium hydroxide (2.31 g, 12.7 mmol) and (BOC)₂O (2.92 mL, 12.7 mmol).

B12** (S)-2-[(tert-butoxycarbonyl)amino]-2-(cyclohexyl)acetic acid

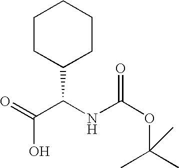

(S)-2-[(tert-butoxycarbonyl)amino]-2-(cyclohexyl)acetic acid (1.20 g, 73%) was isolated as a white foam from (S)-1-amino-1-cyclohexyl carboxylic acid (1.0 g, 6.36 mmol), tetramethylammonium hydroxide (2.31 g, 12.7 mmol) and (BOC)₂O (2.92 mL, 12.7 mmol).

C12** (S)-tert-butyl[1-(2-methylphenylcarbamoyl)-1-cyclohexyl-methyl]-carbamate

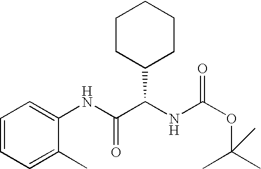

To a screw cap vial was added (S)-2-[(tert-butoxycarbonyl)amino]-2-(cyclohexyl)acetic acid (514.7 mg, 2.00 mmol) and THF (3 mL). The clear solution was cooled to −40° C. and N-methylmorpholine (0.24 mL, 2.20 mmol) was added followed by isobutylchloroformate (0.29 mL, 2.20 mmol). The reaction was allowed to stir for two hours below −30° C., during which time, a white precipitate formed. A solution of N-methylmorpholine (0.26 mL, 2.40 mmol) and o-toluidine (0.26 mL, 2.40 mmol) in THF (2 mL) was added. The reaction was allowed to warm to room temperature over four hours. The reaction mixture was diluted with EtOAc followed by successive washings of water, 1N NaHSO₄, water, 1N NaHCO₃, water and brine. The organic phase was dried over MgSO₄, filtered and concentrated in vacuo. The crude product was washed with hexanes, filtered and dried under vacuum to yield the title compound as a white solid (521.9 mg, 75%).

C12* (R)-tert-butyl[1-(2-methylphenylcarbamoyl)-1-cyclohexyl-methyl]-carbamate

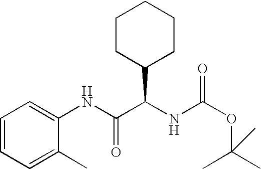

(R)-tert-butyl[1-(2-methylphenylcarbamoyl)-1-cyclohexyl-methyl]-carbamate was isolated as a white solid (465.2 mg, 67%) from (R)-2-[(tert-butoxycarbonyl)amino]-2-(cyclohexyl)acetic acid (514.7 mg, 2.00 mmol), N-methylmorpholine (0.24 mL, 2.20 mmol), isobutylchloroformate (0.29 mL, 2.20 mmol), o-toluidine (0.26 mL, 2.40 mmol) and N-methylmorpholine (0.26 mL, 2.40 mmol).

D12* (R)-2-Amino-N-(2-methylphenyl)-2-cyclohexylacetamide

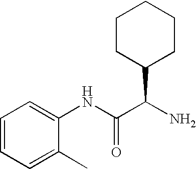

(R)-2-Amino-N-(2-methylphenyl)-2-cyclohexylacetamide (298.7 mg, 90%) was isolated as an off-white solid from (R)-tert-butyl[1-(2-methylphenylcarbamoyl)-1-cyclohexyl-methyl]-carbamate (465.2 mg, 1.34 mmol) and formic acid (96%) (5 mL).

D12** (S)-2-Amino-N-(2-methylphenyl)-2-cyclohexylacetamide

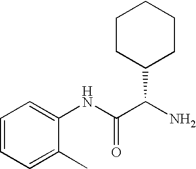

(S)-2-Amino-N-(4-methylphenyl)-2-cyclohexylacetamide (288.2 mg, 77%) was isolated as an off-white solid from (S)-tert-butyl[1-(2-methylphenylcarbamoyl)-1-cyclohexyl-methyl]-carbamate (521.9 mg, 1.51 mmol) and formic acid (96%) (5 mL).

E12.1** (S)-N-(2-methylphenyl)-2-[3-(2-nitrophenyl)-thioureido]-2-cyclohexyl acetamide

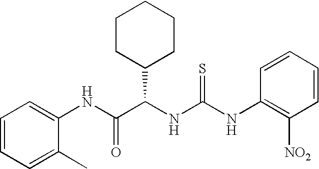

(S)-N-(2-methylphenyl)-2-[3-(2-nitrophenyl)-thioureido]-2-cyclohexyl acetamide (8.4 mg, 20%) was isolated as a yellow solid from (S)-2-Amino-N-(4-methylphenyl)-2-cyclohexylacetamide (24.6 mg, 0.10 mmol) and 2-nitrophenylisothiocyanate (23.4 mg, 0.13 mmol).

E12.1* (R)-N-(2-methylphenyl)-2-[3-(2-nitrophenyl)-thioureido]-2-cyclohexyl acetamide

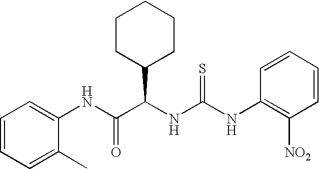

(R)-N-(2-methyl phenyl)-2-[3-(2-nitrophenyl)-thioureido]-2-cyclohexyl acetamide (10.0 mg, 23%) was isolated as a yellow solid from (R)-2-Amino-N-(4-methylphenyl)-2-cyclohexylacetamide (24.6 mg, 0.10 mmol) and 2-nitrophenylisothiocyanate (23.4 mg, 0.13 mmol).

E12.2** (S)-N-(2-methylphenyl)-2-[3-(4-methoxycarbonylphenyl)-thioureido]-2-cyclohexyl acetamide

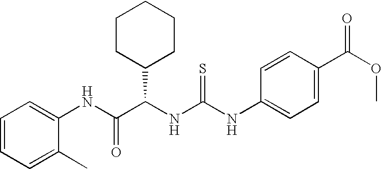

(S)-N-(2-methylphenyl)-2-[3-(4-methoxycarbonyl)-thioureido]-2-cyclohexyl acetamide (32.4 mg, 74%) was isolated as a white solid from (S)-2-Amino-N-(4-methylphenyl)-2-cyclohexylacetamide (24.6 mg, 0.10 mmol) and 4-methoxycarbonylphenylisothiocyanate (23.2 mg, 0.12 mmol).

E12.2* (R)-N-(2-methylphenyl)-2-[3-(4-methoxycarbonylphenyl)-thioureido]-2-cyclohexyl acetamide

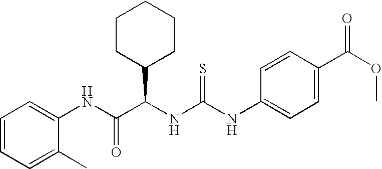

(R)-N-(2-methyl phenyl)-2-[3-(4-methoxycarbonylphenyl)-thioureido]-2-cyclohexyl acetamide (32.8 mg, 75%) was isolated as a white solid from (R)-2-Amino-N-(4-methylphenyl)-2-cyclohexylacetamide (24.6 mg, 0.10 mmol) and 4-methoxycarbonylphenylisothiocyanate (23.2 mg, 0.12 mmol).

### Example 13

B13 BOC-DL-Leucine

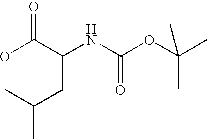

BOC-DL-Leucine (1.82 g, 90%) was isolated as a white solid from DL-Leucine (1.14 g, 8.69 mmol), tetramethylammonium hydroxide (1.57 g, 8.69 mmol) and (BOC)₂O (4.00 mL, 17.4 mmol).

C13 Tert-butyl[1-(2-methylphenylcarbamoyl)-1-(2-methyl-propyl)methyl]-carbamate

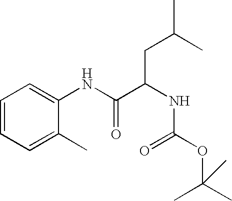

Tert-butyl[1-(2-methylphenylcarbamoyl)-1-(2-methyl-propyl)methyl]-carbamate (479.0 mg, 75%) was isolated as a white solid from BOC-DL-Leucine (462.6 mg, 2.0 mmol), N-methylmorpholine (0.24 mL, 2.20 mmol), isobutylchloroformate (0.29 mL, 2.20 mmol), o-toluidine (0.26 mL, 2.40 mmol) and N-methylmorpholine (0.26 mL, 2.40 mmol).

D13 2-Amino-N-(2-methylphenyl)-2-(2-methylpropyl)acetamide

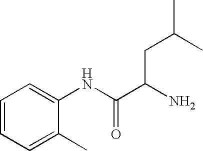

2-Amino-N-(2-methylphenyl)-2-(2-methylpropyl)acetamide (280.2 mg, 85%) was isolated as a white solid from Tert-butyl[1-(2-methylphenylcarbamoyl)-1-(2-methyl-propyl)methyl]-carbamate (479.0 mg, 1.49 mmol).

E13.1 N-(2-methylphenyl)-2-[3-(2-nitrophenyl)-thioureido]-2-(2-methyl propyl)acetamide

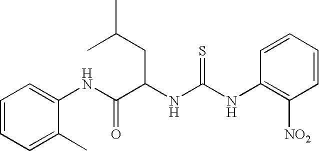

N-(2-methylphenyl)-2-[3-(2-nitrophenyl)-thioureido]-2-(2-methyl propyl)acetamide (14.9 mg, 37%) was isolated as a light yellow solid from 2-Amino-N-(2-methylphenyl)-2-(2-methylpropyl)acetamide (22.0 mg, 0.10 mmol) and 2-nitrophenylisothiocyanate (23.4 mg, 0.13 mmol).

E13.2 N-(2-methylphenyl)-2-[3-(4-methoxycarbonylphenyl)-thioureido]-2-(2-methyl propyl)acetamide

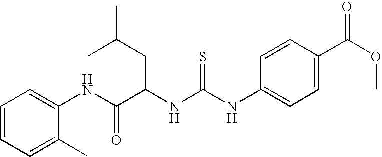

N-(2-methylphenyl)-2-[3-(4-methoxycarbonylphenyl)-thioureido]-2-(2-methyl propyl)acetamide (39.7 mg, 96%) was isolated as a white solid from 2-Amino-N-(2-methylphenyl)-2-(2-methylpropyl)acetamide (22.0 mg, 0.10 mmol) and 4-methoxycarbonylphenylisothiocyanate (23.2 mg, 0.12 mmol).

### Example 14

B14 [tert-butoxycarbonyl)methylamino](phenyl)acetic acid

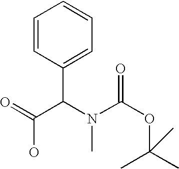

[tert-butoxycarbonyl)methylamino](phenyl)acetic acid (1.42 g, 88%) was isolated as a white solid from N-methylphenylglycine (1.0 g, 6.05 mmol), tetramethylammonium hydroxide (1.10 g, 6.05 mmol) and (BOC)₂O (2.78 mL, 12.1 mmol).

C14 tert-butyl[1-(2-methylphenylcarbamoyl)-1-phenyl-methyl]-N-methylcarbamate

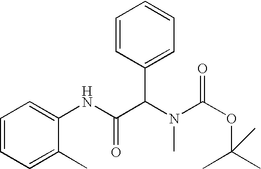

tert-butyl[1-(2-methylphenylcarbamoyl)-1-phenyl-methyl]-N-methylcarbamate was isolated as a white solid (93.1 mg, 35%) from [tert-butoxycarbonyl)methylamino](phenyl)acetic acid (199.0 mg, 0.75 mmol), N-methylmorpholine (0.09 mL, 0.83 mmol), isobutylchloroformate (0.11 mL, 0.83 mmol), o-toluidine (0.10 mL, 0.90 mmol) and N-methylmorpholine (0.10 mL, 0.90 mmol).

D14 2-methylamino-N-(2-methylphenyl)-2-phenylacetamide

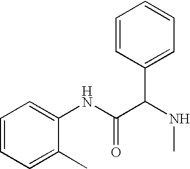

2-methylamino-N-(2-methylphenyl)-2-phenylacetamide was isolated (57.6 mg, 91%) from tert-butyl[1-(2-methylphenylcarbamoyl)-1-phenyl-methyl]-N-methylcarbamate (88.1 mg, 0.25 mmol).

E14.1 N-(2-methylphenyl)-2-[3-(2-nitrophenyl)-1-N-methyl-thioureido]-2-phenyl acetamide

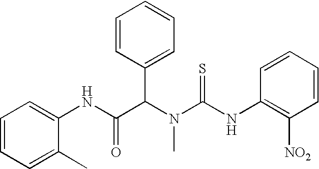

N-(2-methylphenyl)-2-[3-(2-nitrophenyl)-1-N-methyl-thioureido]-2-phenyl acetamide was isolated as a yellow solid (16.0 mg, 53%) from 2-methylamino-N-(4-methylphenyl)-2-phenylacetamide (18.2 mg, 0.07 mmol) and 2-nitrophenylisothiocyanate (16.8 mg, 0.09 mmol).

E14.2 N-(2-methylphenyl)-2-[3-(2-trifluoromethylphenyl)-1-N-methyl-thioureido]-2-phenyl acetamide

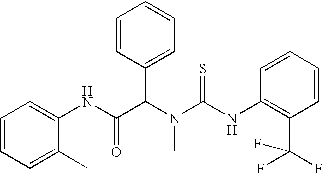

N-(2-methylphenyl)-2-[3-(2-trifluoromethylphenyl)-1-N-methyl-thioureido]-2-phenyl acetamide was isolated as a white solid (26.2 mg, 82%) from 2-methylamino-N-(4-methylphenyl)-2-phenylacetamide (18.2 mg, 0.07 mmol) and 2-trifluoromethylphenylisothiocyanate (18.9 mg, 0.09 mmol).

E14.3 N-(2-methylphenyl)-2-[3-(4-methoxy-2-nitrophenyl)-1-N-methyl-thioureido]-2-phenyl acetamide

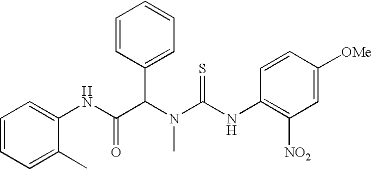

N-(2-methylphenyl)-2-[3-(4-methoxy-2-nitrophenyl)-1-N-methyl-thioureido]-2-phenyl acetamide was isolated as an orange solid (20.8 mg, 64%) from 2-methylamino-N-(4-methylphenyl)-2-phenylacetamide (18.2 mg, 0.07 mmol) and 4-methoxy-2-nitro-phenylisothiocyanate (19.6 mg, 0.09 mmol).

### Example 15

C15 tert-butyl[1-(2-methoxycarbonylphenylcarbamoyl)-1-phenyl-methyl]-carbamate

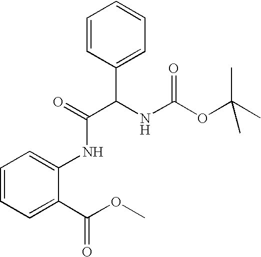

tert-butyl[1-(2-methoxycarbonylphenylcarbamoyl)-1-phenyl-methyl]-carbamate was isolated as a white solid (162.2 mg, 56%) from 2-tert-butoxycarbonylaminophenylacetic acid (188.5 mg, 0.75 mmol), N-methylmorpholine (0.09 mL, 0.83 mmol), isobutylchloroformate (0.11 mL, 0.83 mmol), methylanthranilate (0.12 mL, 0.90 mmol) and N-methylmorpholine (0.10 mL, 0.90 mmol).

D15 2-Amino-N-(2-methoxycarbonylphenyl)-2-phenylacetamide

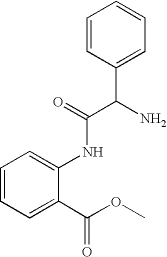

2-Amino-N-(2-methoxycarbonylphenyl)-2-phenylacetamide (98.3 mg, 86%) was isolated from tert-butyl[1-(2-methoxycarbonylphenylcarbamoyl)-1-phenyl-methyl]-N-methylcarbamate (155.0 mg, 0.40 mmol) and formic acid (2 mL).

E15.1 N-(2-methoxycarbonylphenyl)-2-[3-(2-nitrophenyl)-thioureido]-2-phenyl acetamide

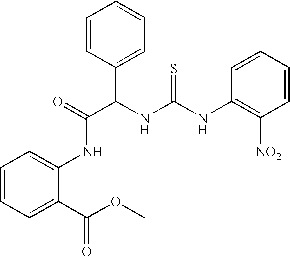

N-(2-methoxycarbonylphenyl)-2-[3-(2-nitrophenyl)-thioureido]-2-phenyl acetamide was isolated as a yellow solid (34.1 mg, 73%) from 2-Amino-N-(2-methoxycarbonylphenyl)-2-phenylacetamide (27.6 mg, 0.10 mmol) and 2-nitrophenylisothiocyanate (22.7 mg, 0.13 mmol).

E15.2 N-(2-methoxycarbonylphenyl)-2-[3-(2-nitro-4-methoxyphenyl)-thioureido]-2-phenyl acetamide

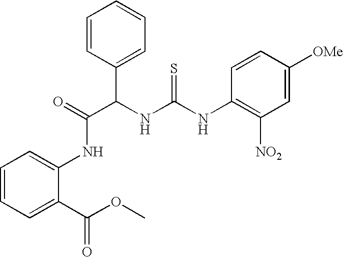

N-(2-methoxycarbonylphenyl)-2-[3-(2-nitro-4-methoxyphenyl)-thioureido]-2-phenyl acetamide was isolated as a yellow solid (34.9 mg, 71%) from 2-Amino-N-(2-methoxycarbonylphenyl)-2-phenylacetamide (27.6 mg, 0.10 mmol) and 4-methoxy-2-nitrophenylisothiocyanate (26.5 mg, 0.13 mmol).

E15.3 N-(2-methoxycarbonylphenyl)-2-[3-(2-trifluoromethylphenyl)-thioureido]-2-phenyl acetamide

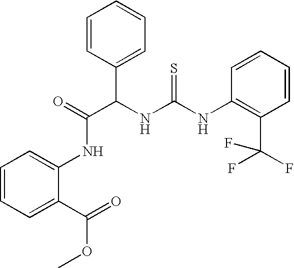

N-(2-methoxycarbonylphenyl)-2-[3-(2-trifluoromethylphenyl)-thioureido]-2-phenyl acetamide was isolated as a white solid (43.9 mg, 90%) from 2-Amino-N-(2-methoxycarbonylphenyl)-2-phenylacetamide (27.6 mg, 0.10 mmol) and 2-trifluoromethylphenylisothiocyanate (25.6 mg, 0.13 mmol).

### Example 16

C16 tert-butyl[1-(2-cyanophenylcarbamoyl)-1-phenyl-methyl]-carbamate

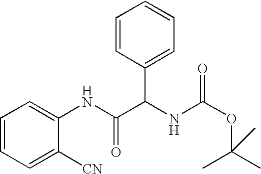

tert-butyl[1-(2-cyanophenylcarbamoyl)-1-phenyl-methyl]-carbamate was isolated as a white solid (47.9 mg, 18%) from tert-butoxy carbonyl phenyl glycine (188.5 mg, 0.75 mmol), N-methylmorpholine (0.09 mL, 0.83 mmol), isobutylchloroformate (0.11 mL, 0.83 mmol), anthranilonitrile (106.3 mg, 0.90 mmol) and N-methylmorpholine (0.10 mL, 0.90 mmol).

D16 2-Amino-N-(2-cyanophenyl)-2-phenylacetamide

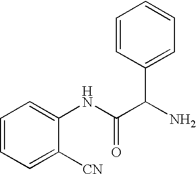

2-Amino-N-(2-cyanophenyl)-2-phenylacetamide was isolated (24.2 mg, 69%) from tert-butyl[1-(2-cyanophenylcarbamoyl)-1-phenyl-methyl]-carbamate (47.9 mg, 0.14 mmol).

E16.1 N-(2-cyanophenyl)-2-[3-(2-nitrophenyl)-thioureido]-2-phenyl acetamide

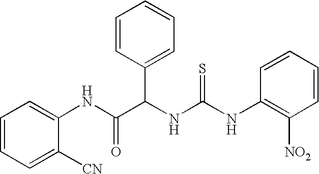

N-(2-cyanophenyl)-2-[3-(2-nitrophenyl)-thioureido]-2-phenyl acetamide was isolated as a yellow solid (30.0 mg, 77%) from 2-Amino-N-(2-cyanophenyl)-2-phenylacetamide (21.5 mg, 0.09 mmol) and 2-nitrophenylisothiocyanate (20.0 mg, 0.11 mmol).

### Example 17

C17 tert-butyl[1-(2-methoxyphenylcarbamoyl)-1-phenyl-methyl]-carbamate

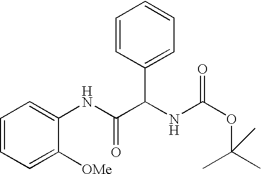

tert-butyl[1-(2-methoxyphenylcarbamoyl)-1-phenyl-methyl]-carbamate was isolated as a light brown solid (159.2 mg, 60%) from tert-butoxycarbonylphenylglycine (188.5 mg, 0.75 mmol), N-methylmorpholine (0.09 mL, 0.83 mmol), isobutylchloroformate (0.11 mL, 0.83 mmol), o-anisidine (0.10 mL, 0.90 mmol) and N-methylmorpholine (0.10 mL, 0.90 mmol).

D17 2-Amino-N-(2-methoxyphenyl)-2-phenylacetamide

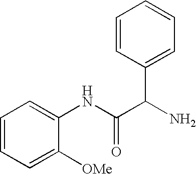

2-Amino-N-(2-methoxyphenyl)-2-phenylacetamide was isolated (92.1 mg, 84%) from tert-butyl[1-(2-methoxyphenylcarbamoyl)-1-phenyl-methyl]-carbamate (154.4 mg, 0.43 mmol) and formic acid (2 mL).

E17.1 N-(2-methoxyphenyl)-2-[3-(2-nitrophenyl)-thioureido]-2-phenyl acetamide

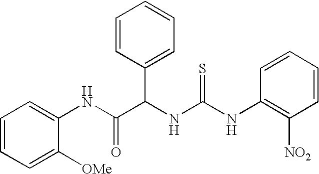

N-(2-methoxyphenyl)-2-[3-(2-nitrophenyl)-thioureido]-2-phenyl acetamide was isolated as a yellow solid (33.2 mg, 69%) from 2-Amino-N-(2-methoxyphenyl)-2-phenylacetamide (28.2 mg, 0.11 mmol) and 2-nitrophenylisothiocyanate (20.0 mg, 0.11 mmol).

E17.2 N-(2-methoxyphenyl)-2-[3-(2-nitro-4-methoxyphenyl)-thioureido]-2-phenyl acetamide

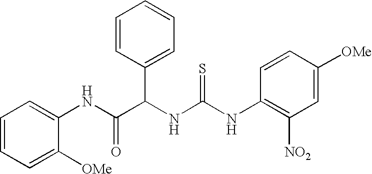

N-(2-methoxyphenyl)-2-[3-(2-nitro-4-methoxyphenyl)-thioureido]-2-phenyl acetamide was isolated as an orange solid (40.8 mg, 80%) from 2-Amino-N-(2-methoxyphenyl)-2-phenylacetamide (28.2 mg, 0.11 mmol) and 4-methoxy-2-nitrophenylisothiocyanate (30.0 mg, 0.14 mmol).

E17.3 N-(2-methoxyphenyl)-2-[3-(2-trifluoromethylphenyl)-thioureido]-2-phenyl acetamide

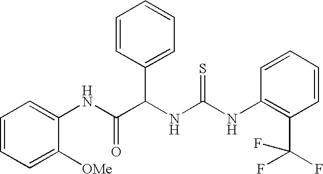

N-(2-methoxyphenyl)-2-[3-(2-trifluoromethylphenyl)-thioureido]-2-phenyl acetamide was isolated as a white solid (45.5 mg, 90%) from 2-Amino-N-(2-methoxyphenyl)-2-phenylacetamide (28.2 mg, 0.11 mmol) and 2-trifluoromethylphenylisothiocyanate (29.0 mg, 0.14 mmol).

### Example 18

C18 tert-butyl[1-(2-methylmercaptophenylcarbamoyl)-1-phenyl-methyl]-carbamate

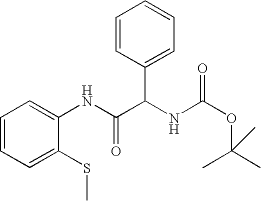

tert-butyl[1-(2-methylmercaptophenylcarbamoyl)-1-phenyl-methyl]-carbamate was isolated as a yellow solid (184.0 mg, 66%) from tert-butoxycarbonylphenylglycine (188.5 mg, 0.75 mmol), N-methylmorpholine (0.09 mL, 0.83 mmol), isobutylchloroformate (0.11 mL, 0.83 mmol), 2-(methylmercapto)aniline (0.11 mL, 0.90 mmol) and N-methylmorpholine (0.10 mL, 0.90 mmol).

D18 2-Amino-N-(2-methylmercaptophenyl)-2-phenylacetamide

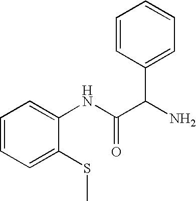

2-Amino-N-(2-methylmercaptophenyl)-2-phenylacetamide was isolated (98.0 mg, 78%) from tert-butyl[1-(2-methylmercaptophenylcarbamoyl)-1-phenyl-methyl]-carbamate (170.5 mg, 0.46 mmol).

E18.1 N-(2-methylmercaptophenyl)-2-[3-(2-nitrophenyl)-thioureido]-2-phenyl acetamide

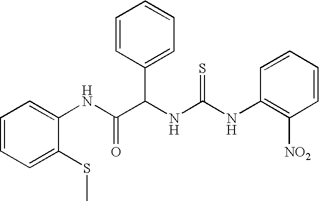

N-(2-methylmercaptophenyl)-2-[3-(2-nitrophenyl)-thioureido]-2-phenyl acetamide was isolated as a yellow solid (30.6 mg, 61%) from 2-Amino-N-(2-methylmercaptophenyl)-2-phenylacetamide (28.6 mg, 0.11 mmol) and 2-nitrophenylisothiocyanate (24.6 mg, 0.14 mmol).

E18.2 N-(2-methylmercaptophenyl)-2-[3-(2-nitro-4-methoxyphenyl)-thioureido]-2-phenyl acetamide

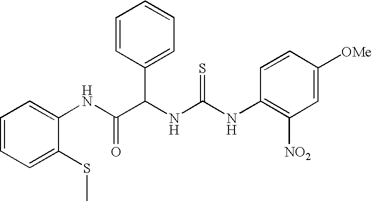

N-(2-methylmercaptophenyl)-2-[3-(2-nitro-4-methoxyphenyl)-thioureido]-2-phenyl acetamide was isolated as a yellow solid (42.1 mg, 79%) from 2-Amino-N-(2-methylmercaptophenyl)-2-phenylacetamide (28.6 mg, 0.11 mmol) and 4-methoxy-2-nitrophenylisothiocyanate (24.6 mg, 0.14 mmol).

E18.3 N-(2-methylmercaptophenyl)-2-[3-(2-trifluoromethylphenyl)-thioureido]-2-phenyl acetamide

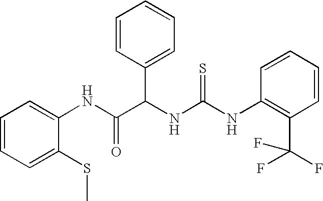

N-(2-methylmercaptophenyl)-2-[3-(2-trifluoromethylphenyl)-thioureido]-2-phenyl acetamide was isolated as a white solid (42.9 mg, 82%) from 2-Amino-N-(2-methylmercaptophenyl)-2-phenylacetamide (28.6 mg, 0.11 mmol) and 2-trifluoromethylphenylisothiocyanate (27.8 mg, 0.14 mmol).

### Example 19

C19 tert-butyl[1-(2-chlorophenylcarbamoyl)-1-phenyl-methyl]-carbamate

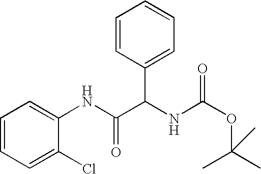

tert-butyl[1-(2-chlorophenylcarbamoyl)-1-phenyl-methyl]-carbamate was isolated as a white solid (208.9 mg, 58%) from N-Tert-butoxycarbonyl DL-phenyl glycine (250.0 mg, 0.99 mmol), N-methylmorpholine (0.12 mL, 1.09 mmol), isobutylchloroformate (0.14 mL, 1.09 mmol), 2-chloroaniline (0.13 mL, 1.19 mmol) and N-methylmorpholine (0.13 mL, 1.19 mmol).

D19 2-Amino-N-(2-chlorophenyl)-2-phenylacetamide

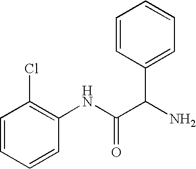

2-Amino-N-(2-chlorophenyl)-2-phenylacetamide was isolated as a white solid (55.7 mg, 35%) from tert-butyl[1-(2-chlorophenylcarbamoyl)-1-phenyl-methyl]-carbamate (202.4 mg, 0.62 mmol).

E19.1 N-(2-chlorophenyl)-2-[3-(2-nitrophenyl)-thioureido]-2-phenyl acetamide

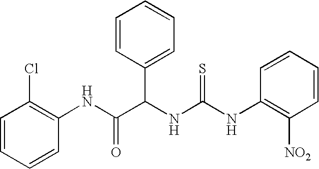

N-(2-chlorophenyl)-2-[3-(2-nitrophenyl)-thioureido]-2-phenyl acetamide was isolated as a yellow solid (17.0 mg, 64%) from 2-Amino-N-(2-chlorophenyl)-2-phenylacetamide (15.0 mg, 0.06 mmol) and 2-nitrophenylisothiocyanate (13.5 mg, 0.07 mmol).

E19.2 N-(2-chlorophenyl)-2-[3-(4-methoxy-2-nitrophenyl)-thioureido]-2-phenyl acetamide

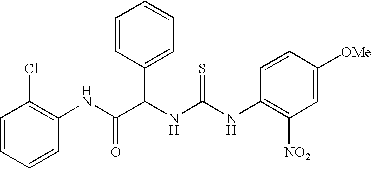

N-(2-chlorophenyl)-2-[3-(4-methoxy-2-nitrophenyl)-thioureido]-2-phenyl acetamide was isolated as a yellow solid (17.6 mg, 62%) from 2-Amino-N-(2-chlorophenyl)-2-phenylacetamide (15.0 mg, 0.06 mmol) and 4-methoxy-2-nitro phenylisothiocyanate (15.7 mg, 0.07 mmol).

E19.3 N-(2-chlorophenyl)-2-[3-(2-trifluoromethylphenyl)-thioureido]-2-phenyl acetamide

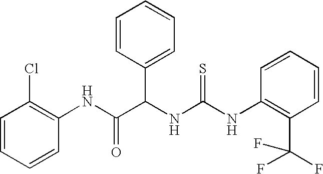

N-(2-chlorophenyl)-2-[3-(2-trifluoromethylphenyl)-thioureido]-2-phenyl acetamide was isolated as a white solid (14.8 mg, 53%) from 2-Amino-N-(2-chlorophenyl)-2-phenylacetamide (15.0 mg, 0.06 mmol) and 2-trifluoromethylphenylisothiocyanate (15.2 mg, 0.07 mmol).

### Example 20

C20 tert-butyl[1-(3-chlorophenylcarbamoyl)-1-phenyl-methyl]-carbamate

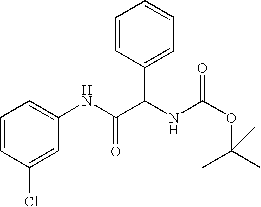

tert-butyl[1-(3-chlorophenylcarbamoyl)-1-phenyl-methyl]-carbamate was isolated as a white solid (289.5 mg, 81%) from N-Tert-butoxycarbonyl DL-phenyl glycine (250.0 mg, 0.99 mmol), N-methylmorpholine (0.12 mL, 1.09 mmol), isobutylchloroformate (0.14 mL, 1.09 mmol), 3-chloroaniline (0.13 mL, 1.19 mmol) and N-methylmorpholine (0.13 mL, 1.19 mmol).

D20 2-Amino-N-(3-chlorophenyl)-2-phenylacetamide

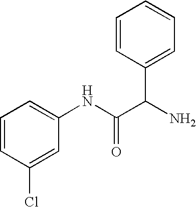

2-Amino-N-(3-chlorophenyl)-2-phenylacetamide was isolated as a white solid (138.6 mg, 62%) from tert-butyl[1-(4-chlorophenylcarbamoyl)-1-phenyl-methyl]-carbamate (280.2 mg, 0.86 mmol).

E20.1 N-(3-chlorophenyl)-2-[3-(2-nitrophenyl)-thioureido]-2-phenyl acetamide

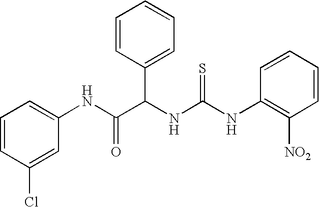

N-(3-chlorophenyl)-2-[3-(2-nitrophenyl)-thioureido]-2-phenyl acetamide was isolated as a yellow solid (13.0 mg, 37%) from 2-Amino-N-(3-chlorophenyl)-2-phenylacetamide (20.0 mg, 0.08 mmol) and 2-nitrophenylisothiocyanate (18.0 mg, 0.10 mmol).

E20.2 N-(3-chlorophenyl)-2-[3-(2-trifluoromethylphenyl)-thioureido]-2-phenyl acetamide

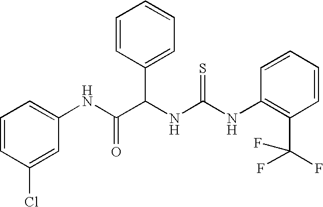

N-(3-chlorophenyl)-2-[3-(2-trifluoromethylphenyl)-thioureido]-2-phenyl acetamide was isolated as a white solid (26.8 mg, 72%) from 2-Amino-N-(3-chlorophenyl)-2-phenylacetamide (20.0 mg, 0.08 mmol) and 2-trifluoromethylphenylisothiocyanate (20.3 mg, 0.10 mmol).

D20.3 N-(3-chlorophenyl)-2-[3-(4-methoxy-2-nitrophenyl)-thioureido]-2-phenyl acetamide

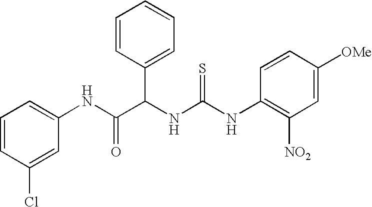

N-(3-chlorophenyl)-2-[3-(4-methoxy-2-nitrophenyl)-thioureido]-2-phenyl acetamide was isolated as an orange solid (15.0 mg, 40%) from 2-Amino-N-(3-chlorophenyl)-2-phenylacetamide (20.0 mg, 0.08 mmol) and 4-methoxy-2-nitrophenylisothiocyanate (21.0 mg, 0.10 mmol).

### Example 21

C21 tert-butyl[1-(4-chlorophenylcarbamoyl)-1-phenyl-methyl]-carbamate

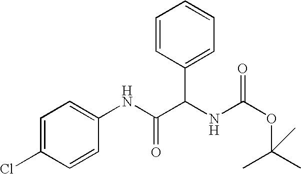

tert-butyl[1-(4-chlorophenylcarbamoyl)-1-phenyl-methyl]-carbamate was isolated as a white solid (266.2 mg, 74%) from N-Tert-butoxycarbonyl DL-phenyl glycine (250.0 mg, 0.99 mmol), N-methylmorpholine (0.12 mL, 1.09 mmol), isobutylchloroformate (0.14 mL, 1.09 mmol), 4-chloroaniline 9152.3 mg, 1.19 mmol) and N-methylmorpholine (0.13 mL, 1.19 mmol).

D21 2-Amino-N-(4-chlorophenyl)-2-phenylacetamide

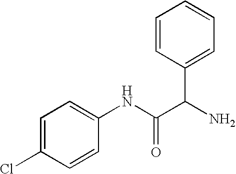

2-Amino-N-(4-chlorophenyl)-2-phenylacetamide was isolated as a white solid (150.3 mg, 72%) from tert-butyl[1-(4-chlorophenylcarbamoyl)-1-phenyl-methyl]-carbamate (259.6 mg, 0.80 mmol).

E21.1 N-(4-chlorophenyl)-2-[3-(2-nitrophenyl)-thioureido]-2-phenyl acetamide

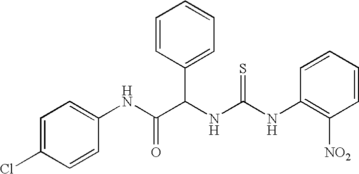

N-(4-chlorophenyl)-2-[3-(2-nitrophenyl)-thioureido]-2-phenyl acetamide was isolated as a light yellow solid (28.3 mg, 80%) from 2-Amino-N-(4-chlorophenyl)-2-phenylacetamide (20.0 mg, 0.08 mmol) and 2-nitrophenylisothiocyanate (18.0 mg, 0.10 mmol).

E21.2 N-(4-chlorophenyl)-2-[3-(2-trifluoromethylphenyl)-thioureido]-2-phenyl acetamide

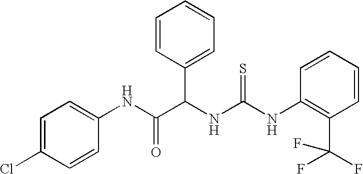

N-(4-chlorophenyl)-2-[3-(2-trifluoromethylphenyl)-thioureido]-2-phenyl acetamide was isolated as a white solid (26.0 mg, 70%) from 2-Amino-N-(4-chlorophenyl)-2-phenylacetamide (20.0 mg, 0.08 mmol) and 2-trifluoromethylisothiocyanate (20.3 mg, 0.10 mmol).

E21.3 N-(4-chlorophenyl)-2-[3-(4-methoxy-2-nitrophenyl)-thioureido]-2-phenyl acetamide

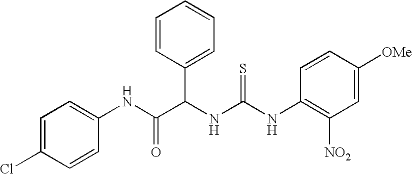

N-(4-chlorophenyl)-2-[3-(4-methoxy-2-nitrophenyl)-thioureido]-2-phenyl acetamide was isolated as an orange solid (14.8 mg, 39%) from 2-Amino-N-(4-chlorophenyl)-2-phenylacetamide (20.0 mg, 0.08 mmol) and 4-methoxy-2-nitrophenylisothiocyanate (21.0 mg, 0.10 mmol).

### Example 22

C22 tert-butyl[1-(2,3-dimethylphenylcarbamoyl)-1-phenyl-methyl]-carbamate

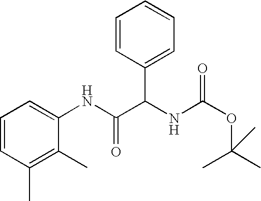

tert-butyl[1-(2,3-dimethylphenylcarbamoyl)-1-phenyl-methyl]-carbamate was isolated as a white solid (230.5 mg, 66%) from BOC-phenylglycine (250.0 mg, 0.99 mmol), N-methylmorpholine (0.12 mL, 1.09 mmol), isobutylchloroformate (0.14 mL, 1.09 mmol), 2,3-dimethylaniline (0.15 mL, 1.19 mmol) and N-methylmorpholine (0.13 mL, 1.19 mmol).

D22 2-Amino-N-(2,3-dimethylphenyl)-2-phenylacetamide

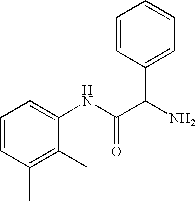

2-Amino-N-(2,3-dimethylphenyl)-2-phenylacetamide was isolated as a white solid (118.6 mg, 75%) from tert-butyl[1-(2,3-dimethylphenylcarbamoyl)-1-phenyl-methyl]-carbamate (220.4 mg, 0.62 mmol).

E22.1 N-(2.3-dimethyl phenyl)-2-[3-(2-nitrophenyl)-thioureido]-2-phenyl acetamide

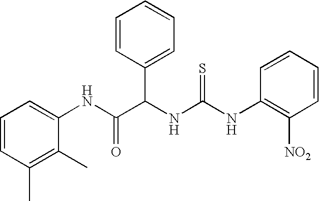

N-(2,3-dimethylphenyl)-2-[3-(2-nitrophenyl)-thioureido]-2-phenyl acetamide was isolated as a yellow solid (19.0 mg, 73%) from 2-Amino-N-(2,3-dimethylphenyl)-2-phenylacetamide (14.6 mg, 0.06 mmol) and 2-nitrophenylisothiocyanate (13.5 mg, 0.07 mmol).

E22.2 N-(2.3-dimethylphenyl)-2-[3-(2-trifluoromethylphenyl)-thioureido]-2-phenyl acetamide

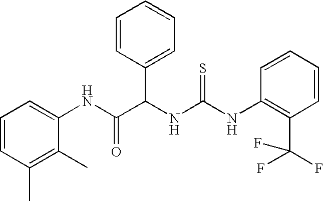

N-(2,3-dimethylphenyl)-2-[3-(2-trifluoromethyl phenyl)-thioureido]-2-phenyl acetamide was isolated as a white solid (17.0 mg, 62%) from 2-Amino-N-(2,3-dimethylphenyl)-2-phenylacetamide (14.6 mg, 0.06 mmol) and 2-trifluoromethylphenylisothiocyanate (15.2 mg, 0.07 mmol).

### Example 23

C23 tert-butyl[1-(5,6,7,8-tetrahydro-1-naphthylcarbamoyl)-1-phenyl-methyl]-carbamate

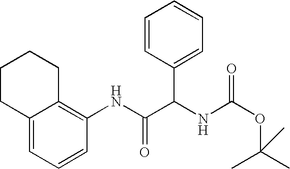

tert-butyl[1-(5,6,7,8-tetrahydro-1-naphthylcarbamoyl)-1-phenyl-methyl]-carbamate was isolated as a white solid (299.8 mg, 80%) from BOC-phenyl glycine (250.0 mg, 0.99 mmol), N-methylmorpholine (0.12 mL, 1.09 mmol), isobutylchloroformate (0.14 mL, 1.09 mmol), 5,6,7,8-tetrahydro-1-naphthlamine (175.8 mg, 1.19 mmol) and n-methylmorpholine (0.13 mL, 1.19 mmol).

D23 2-Amino-N-(5.6,7,8-tetrahydronaphthyl)-2-phenylacetamide

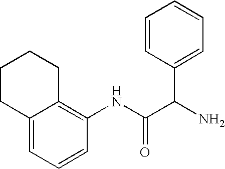

2-Amino-N-(5,6,7,8-tetrahydronaphthyl)-2-phenylacetamide was isolated as a white solid (147.9 mg, 70%) from tert-butyl[1-(5,6,7,8-tetrahydro-1-naphthylcarbamoyl)-1-phenyl-methyl]-carbamate (286.9 mg, 0.75 mmol).

E23.1 N-(5,6,7,8-tetrahydronaphthyl)-2-[3-(2-nitrophenyl)-thioureido]-2-phenyl acetamide

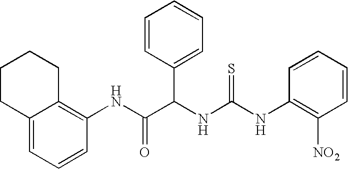

N-(5,6,7,8-tetrahydronaphthyl)-2-[3-(2-nitrophenyl)-thioureido]-2-phenyl acetamide was isolated as a yellow solid (12.6 mg, 46%) from 2-Amino-N-(5,6,7,8,-tetrahydronaphthyl)-2-phenylacetamide (16.1 mg, 0.06 mmol) and 2-nitrophenylisothiocyanate (13.5 mg, 0.07 mmol).

E23.2 N-(5,6,7,8,-tetrahydronaphthyl)-2-[3-(2-trifluoromethylphenyl)-thioureido]-2-phenyl acetamide

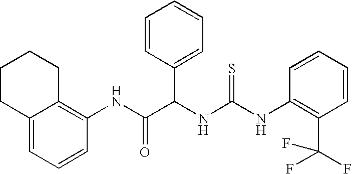

N-(5,6,7,8,-tetrahydronaphthyl)-2-[3-(2-trifluoromethylphenyl)-thioureido]-2-phenyl acetamide was isolated as a white solid (14.5 mg, 50%) from 2-Amino-N-(5,6,7,8,-tetrahydronaphthyl)-2-phenylacetamide (16.1 mg, 0.06 mmol) and 2-trifluoromethylphenylisothiocyanate (15.2 mg, 0.07 mmol).

### Example 24

C24 tert-butyl[1-(2-methyl-4-chlorophenylcarbamoyl)-1-phenyl-methyl]-carbamate

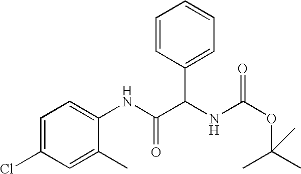

tert-butyl[1-(2-methyl-4-chlorophenylcarbamoyl)-1-phenyl-methyl]-carbamate was isolated as white solid (296.7 mg, 80%) from BOC-phenylglycine (250.0 mg, 0.99 mmol), N-methylmorpholine (0.12 mL, 1.09 mmol), isobutylchloroformate (0.14 mL, 1.09 mmol), -2-methyl-4-chloroaniline (169.1 mg, 1.19 mmol) and N-methylmorpholine (0.13 mL, 1.19 mmol).

D24 2-Amino-N-(2-methyl-4-chlorophenyl)-2-phenylacetamide

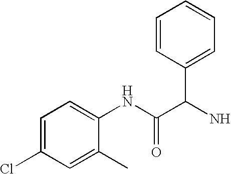

2-Amino-N-(2-methyl-4-chlorophenyl)-2-phenylacetamide was isolated as a white solid (148.3 mg, 71%) from tert-butyl[1-(2-methyl-4-chlorophenylcarbamoyl)-1-phenyl-methyl]-carbamate (284.6 mg, 0.76 mmol).

E24.1 N-(2-methyl-4-chlorophenyl)-2-[3-(2-nitrophenyl)-thioureido]-2-phenyl acetamide

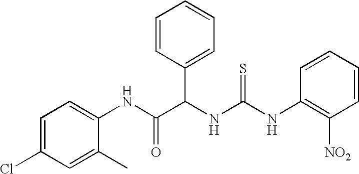

N-(2-methyl-4-chlorophenyl)-2-[3-(2-nitrophenyl)-thioureido]-2-phenyl acetamide was isolated as a yellow solid (18.0 mg, 66%) from 2-Amino-N-(2-methyl-4-chlorophenyl)-2-phenylacetamide (15.8 mg, 0.06 mmol) and 2-nitrophenylisothiocyanate (13.5 mg, 0.07 mmol).

E24.2 N-(2-methyl-4-chlorophenyl)-2-[3-(2-trifluoromethylphenyl)-thioureido]-2-phenyl acetamide

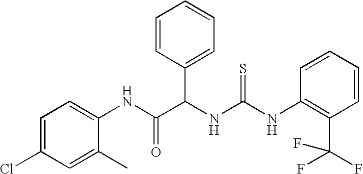

N-(2-methyl-4-chlorophenyl)-2-[3-(2-trifluoromethylphenyl)-thioureido]-2-phenyl acetamide was isolated as a white solid (13.3 mg, 46%) from 2-Amino-N-(2-methyl-4-chlorophenyl)-2-phenylacetamide (15.8 mg, 0.06 mmol) and 2-trifluoromethylphenylisothiocyanate (15.2 mg, 0.07 mmol).

### Example 25

C25 tert-butyl[1-(5-phenyl-5-toluidinyl carbamoyl)-1-phenyl-methyl]-carbamate

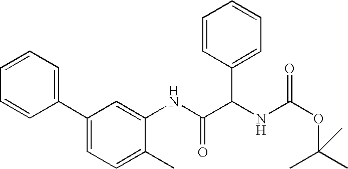

tert-butyl[1-(5-pheny-2-toluidinyl carbamoyl)-1-phenyl-methyl]-carbamate was isolated as a white solid (193.6 mg, 47%) from BOC-phenyl glycine (250.0 mg, 0.99 mmol), N-methylmorpholine (0.12 mL, 1.09 mmol), isobutylchloroformate (0.14 mL, 1.09 mmol), 2-methyl-5-phenyl aniline (218.8 mg, 1.19 mmol) and N-methylmorpholine (0.13 mL, 1.19 mmol).

D25 2-Amino-N-(5-phenyl-2-toluidinyl)-2-phenylacetamide

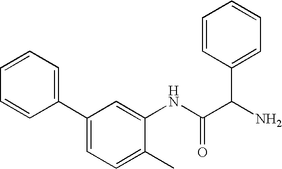

2-Amino-N-(5-phenyl-2-toluidinyl)-2-phenylacetamide was isolated as a white solid (110.0 mg, 79%) from tert-butyl[1-(5-phenyl-2-toluidinyl carbamoyl)-1-phenyl-methyl]-carbamate (184.7 mg, 0.44 mmol).

E25.1 N-(−5-phenyl-2-toluidinyl)-2-[3-(2-nitrophenyl)-thioureido]-2-phenyl acetamide

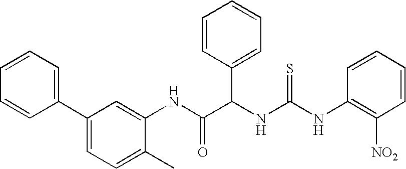

N-(5-phenyl-2-toluidinyl)-2-[3-(2-nitrophenyl)-thioureido]-2-phenyl acetamide was isolated as a yellow solid (19.7 mg, 66%) from 2-Amino-N-(5-phenyl-2-toluidinyl)-2-phenylacetamide (18.2 mg, 0.06 mmol) and 2-nitrophenylisothiocyanate (13.5 mg, 0.07 mmol).

E25.2 N-(5-phenyl-2-toluidinyl)-2-[3-(2-trifluoromethylphenyl)-thioureido]-2-phenyl acetamide

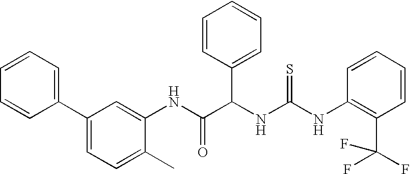

N-(5-phenyl-2-toluidinyl)-2-[3-(2-trifluoromethylphenyl)-thioureido]-2-phenyl acetamide was isolated as a white solid (23.3 mg, 75%) from 2-Amino-N-(5-phenyl-2-toluidinyl)-2-phenylacetamide (18.2 mg, 0.06 mmol) and 2-trifluoromethylphenylisothiocyanate (15.2 mg, 0.07 mmol).

### Example 26

C26 tert-butyl[1-(4-phenyl-2-toluidinyl carbamoyl)-1-phenyl-methyl]-carbamate

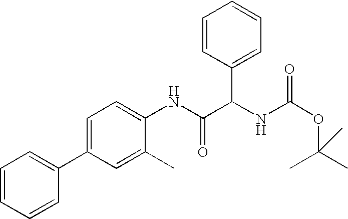

tert-butyl[1-(4-phenyl-2-toluidinylcarbamoyl)-1-phenyl-methyl]-carbamate was isolated as a white solid (315.7 mg, 77%) from BOC-phenyl glycine (250.0 mg, 0.99 mmol), N-methylmorpholine (0.12 mL, 1.09 mmol), isobutylchloroformate (0.14 mL, 1.09 mmol), 4-phenyl-2-methyl aniline (218.8 mg, 1.19 mmol) and N-methylmorpholine (0.13 mL, 1.19 mmol).

D26 2-Amino-N-(4-phenyl-2-toluidinyl)-2-phenylacetamide

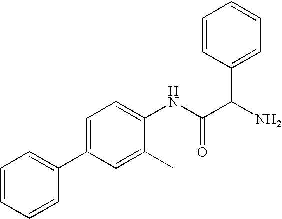

2-Amino-N-(4-phenyl-2-toluidinyl)-2-phenylacetamide was isolated as a white solid (178.3 mg, 77%) from tert-butyl[1-(4-phenyl-2-toluidinylcarbamoyl)-1-phenyl-methyl]-carbamate (303.7 mg, 0.73 mmol).

E26.1 N-(4-phenyl-2-toluidinyl)-2-[3-(2-nitrophenyl)-thioureido]-2-phenyl acetamide

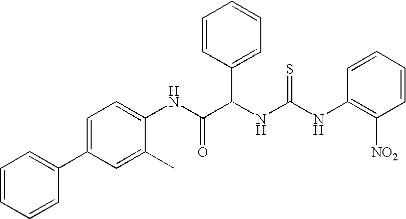

N-(4-phenyl-2-toluidinyl)-2-[3-(2-nitrophenyl)-thioureido]-2-phenyl acetamide was isolated as a yellow solid (19.6 mg, 66%) from 2-Amino-N-(4-phenyl-2-toluidinyl)-2-phenylacetamide (18.2 mg, 0.06 mmol) and 2-nitrophenylisothiocyanate (13.5 mg, 0.07 mmol).

E26.2 N-(4-phenyl-2-toluidinyl)-2-[3-(2-trifluoromethylphenyl)-thioureido]-2-phenyl acetamide

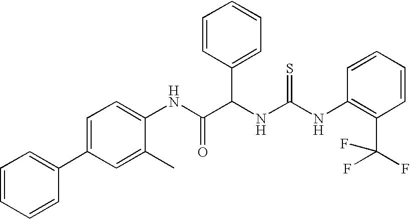

N-(4-phenyl-2-toluidinyl)-2-[3-(2-trifluoromethylphenyl)-thioureido]-2-phenyl acetamide was isolated as a white solid (11.6 mg, 37%) from 2-Amino-N-(4-phenyl-2-toluidinyl)-2-phenylacetamide (18.2 mg, 0.06 mmol) and 2-trifluoromethylphenylisothiocyanate (15.2 mg, 0.07 mmol).

### Example 27

C27 tert-butyl[4-(6-ethyl 2-toluidinylcarbamoyl)-1-phenyl-methyl]-carbamate

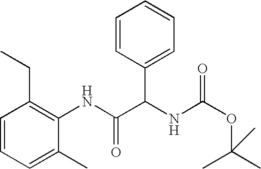

tert-butyl[1-(6-ethyl-2-toluidinylcarbamoyl)-1-phenyl-methyl]-carbamate was isolated as a white solid (236.6 mg, 65%) from BOC-phenylglycine (250.0 mg, 0.99 mmol), N-methylmorpholine (0.12 mL, 1.09), isobutylchloroformate (0.14 mL, 1.09 mmol), 6-ethyl-o-toluidine (0.17 mL, 1.19 mmol) and N-methylmorpholine (0.13 mL, 1.19 mmol).

D27 2-Amino-N-(6-ethyl-2-toluidinyl)-2-phenylacetamide

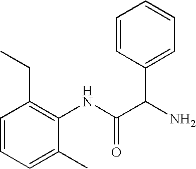

2-Amino-N-(6-ethyl-2-toluidinyl)-2-phenylacetamide was isolated as a white solid (83.5 mg, 50%) from tert-butyl[1-(6-ethyl-2-toluidinylcarbamoyl)-1-phenyl-methyl]-carbamate (228.2 mg, 0.62 mmol).

E27.1 N-(6-ethyl-2-toluidinyl)-2-[3-(4-methoxy-2-nitrophenyl)-thioureido]-2-phenyl acetamide

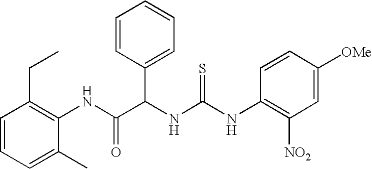

N-(6-ethyl-2-toluidinyl)-2-[3-(4-methoxy-2-nitrophenyl)-thioureido]-2-phenyl acetamide was isolated as a yellow solid (21.0 mg, 73%) from 2-Amino-N-(6-ethyl-2-toluidinyl)-2-phenylacetamide (15.8 mg, 0.06 mmol) and 4-methoxy-2-nitrophenylisothiocyanate (14.9 mg, 0.07 mmol).

### Example 28

C28 tert-butyl[1-(2-isopropyl-6-methylphenylcarbamoyl)-1-phenyl-methyl]-carbamate

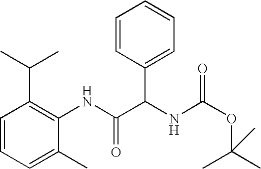

tert-butyl[1-(2-isopropyl-6-methylphenylcarbamoyl)-1-phenyl-methyl]-carbamate was isolated as a white solid (216.1 mg, 57%) from BOC-phenylglycine (250.0 mg, 0.99 mmol), N-methylmorpholine (0.12 mL, 1.09 mmol), isobutylchloroformate (0.14 mL, 1.09 mmol), 2-isopropyl-6-methylaniline (0.19 mL, 1.19 mmol) and N-methylmorpholine (0.13 mL, 1.19 mmol).

### Example 28

D28 2-Amino-N-(2-isopropyl-6-methylphenyl)-2-phenylacetamide

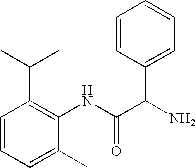

2-Amino-N-(2-isopropyl-6-methylphenyl)-2-phenylacetamide was isolated as a white solid (78.8 mg, 51%) from tert-butyl[1-(2-isopropyl-6-methylphenylcarbamoyl)-1-phenyl-methyl]-carbamate (210.0 mg, 0.55 mmol).

E28.1 N-(2-isopropyl-6-methylphenyl)-2-[3-(4-methoxy-2-nitrophenyl)-thioureido]-2-phenyl acetamide

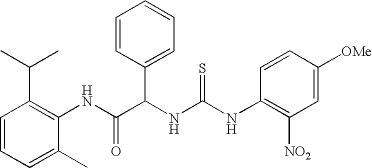

N-(2-isopropyl-6-methylphenyl)-2-[3-(4-methoxy-2-nitrophenyl)-thioureido]-2-phenyl acetamide was isolated as a yellow solid (23.8 mg, 81%) from 2-Amino-N-(2-isopropyl-6-methylphenyl)-2-phenylacetamide (16.7 mg, 0.06 mmol) and 4-methoxy-2-nitrophenylisothiocyanate (14.9 mg, 0.07 mmol).

### Example 29

C29 tert-butyl[1-(2-chloro-6-methylphenylcarbamoyl)-1-phenyl-methyl]-carbamate

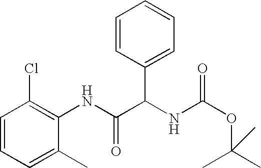

tert-butyl[1-(2-chloro-6-methylphenylcarbamoyl)-1-phenyl-methyl]-carbamate was isolated as a white solid (139.2 mg, 38%) from BOC-phenylglycine (250.0 mg, 0.99 mmol), N-methylmorpholine (0.12 mL, 1.09 mmol), isobutylchloroformate (0.14 mL, 1.09 mmol), 2-chloro-6-methylaniline (0.15 mL, 1.19 mmol) and N-methylmorpholine (0.13 mL, 1.19 mmol).

D29 2-Amino-N-(2-chloro-6-methylphenyl)-2-phenylacetamide

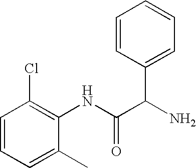

2-Amino-N-(2-chloro-6-methylphenyl)-2-phenylacetamide was isolated as a white solid (37.9 mg, 38%) from tert-butyl[1-(2-chloro-6-methylphenylcarbamoyl)-1-phenyl-methyl]-carbamate (132.8 mg, 0.36 mmol).

E29.1 N-(2-chloro-6-methylphenyl)-2-[3-(4-methoxy-2-nitrophenyl)-thioureido]-2-phenyl acetamide

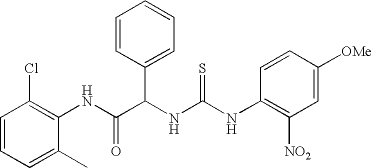

N-(2-chloro-6-methylphenyl)-2-[3-(4-methoxy-2-nitrophenyl)-thioureido]-2-phenyl acetamide was isolated as a yellow solid (10.2 mg, 53%) from 2-Amino-N-(2-chloro-6-methylphenyl)-2-phenylacetamide (10.0 mg, 0.04 mmol) and 4-methoxy-2-nitrophenylisothiocyanate (9.2 mg, 0.04 mmol).

### Example 30

C30 tert-butyl[1-(2,4-dimethylphenylcarbamoyl)-1-phenyl-methyl]-carbamate

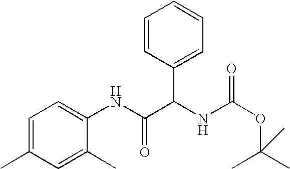

tert-butyl[1-(2,4-dimethylphenylcarbamoyl)-1-phenyl-methyl]-carbamate was isolated as a white solid (240.2 mg, 68%) from BOC-phenylglycine (250.0 mg, 0.99 mmol), N-methylmorpholine (0.12 mL, 1.09 mmol), isobutylchloroformate (0.14 mL, 1.09 mmol), 2,4-dimethylaniline (0.15 mL, 1.19 mmol) and N-methylmorpholine (0.13 mL, 1.19 mmol).

D30 2-Amino-N-(2,4-dimethylphenyl)-2-phenylacetamide

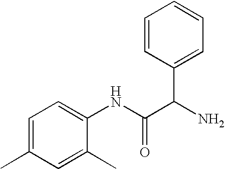

2-Amino-N-(2,4-dimethylphenyl)-2-phenylacetamide was isolated as a white solid (86.1 mg, 52%) from tert-butyl[1-(2,4-dimethylphenylcarbamoyl)-1-phenyl-methyl]-carbamate (231.0 mg, 0.65 mmol).

E30.1 N-(2.4-dimethylphenyl)-2-[3-(4-methoxy-2-nitrophenyl)-thioureido]-2-phenyl acetamide

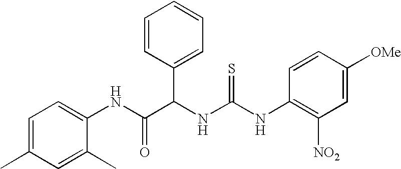

N-(2,4-dimethylphenyl)-2-[3-(4-methoxy-2-nitrophenyl)-thioureido]-2-phenyl acetamide was isolated as a yellow solid (19.5 mg, 70%) from 2-Amino-N-(2,4-dimethylphenyl)-2-phenylacetamide (15.0 mg, 0.06 mmol) and 4-methoxy-2-nitrophenylisothiocyanate (14.9 mg, 0.07 mmol).

### Example 31

C31 tert-butyl[1-(2,5-dimethylphenylcarbamoyl)-1-phenyl-methyl]-carbamate

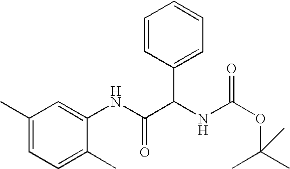

tert-butyl[1-(2,5-dimethylphenylcarbamoyl)-1-phenyl-methyl]-carbamate was isolated as a white solid (98.4 mg, 28%) from BOC-phenylglycine (250.0 mg, 0.99 mmol), N-methylmorpholine (0.12 mL, 1.09 mmol), isobutylchloroformate (0.14 mL, 1.09 mmol), 2,5-dimethylaniline (0.15 mL, 1.19 mmol) and N-methylmorpholine (0.13 mL, 1.19 mmol).

D31 2-Amino-N-(2,5-dimethylphenyl)-2-phenylacetamide

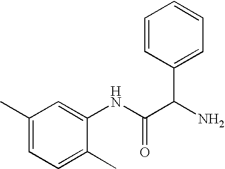

2-Amino-N-(2,5-dimethylphenyl)-2-phenylacetamide was isolated as a white solid (20.0 mg, 28%) from tert-butyl[1-(2,5-dimethylphenylcarbamoyl)-1-phenyl-methyl]-carbamate (90.6 mg, 0.28 mmol).

E31.1 N-(2,5-dimethylphenyl)-2-[3-(4-methoxy-2-nitrophenyl)-thioureido]-2-phenyl acetamide

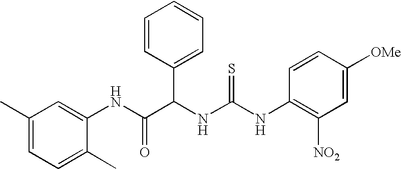

N-(2,5-dimethylphenyl)-2-[3-(4-methoxy-2-nitrophenyl)-thioureido]-2-phenyl acetamide was isolated as a yellow solid (10.2 mg, 73%) from 2-Amino-N-(2,5-dimethylphenyl)-2-phenylacetamide (8.0 mg, 0.03 mmol) and 4-methoxy-2-nitrophenylisothiocyanate (7.9 mg, 0.04 mmol).

### Example 32

C32 tert-butyl[1-(2-methyl-1-naphthylcarbamoyl)-1-phenyl-methyl]-carbamate

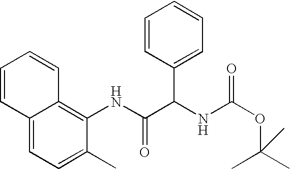

tert-butyl[1-(2-methyl-1-naphthylcarbamoyl)-1-phenyl-methyl]-carbamate was isolated as an off-white solid (165.7 mg, 43%) from BOC-phenyl glycine (250.0 mg, 0.99 mmol), N-methylmorpholine (0.12 mL, 1.09 mmol), isobutylchloroformate (0.14 mL, 1.09 mmol), 2-methyl-1-naphthylamine (187.7 mg, 1.19 mmol) and N-methylmorpholine (0.13 mL, 1.19 mmol).

D32 2-Amino-N-(2-methyl-1-napthyl)-2-phenylacetamide

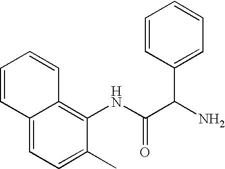

2-Amino-N-(2-methyl-1-napthyl)-2-phenylacetamide was isolated as a thick gel (66.0 mg, 55%) from tert-butyl[1-(2-methyl-1-naphthylcarbamoyl)-1-phenyl-methyl]-carbamate (159.0 mg, 0.41 mmol).

E32.1 N-(2-methyl-1-naphthyl)-2-[3-(4-methoxy-2-nitrophenyl)-thioureido]-2-phenyl acetamide

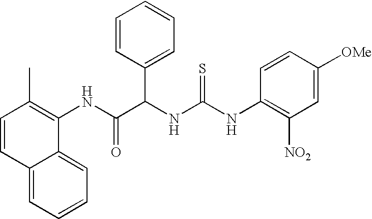

N-(2-methyl-1-naphthyl)-2-[3-(4-methoxy-2-nitrophenyl)-thioureido]-2-phenyl acetamide was isolated as a yellow solid (18.8 mg, 63%) from 2-Amino-N-(2-methyl-1-napthyl)-2-phenylacetamide (17.1 mg, 0.06 mmol) and 4-methoxy-2-nitrophenylisothiocyanate (14.9 mg, 0.07 mmol).

### Example 33

C33 tert-butyl[1-(2,6-dimethylphenylcarbamoyl)-1-phenyl-methyl]-carbamate

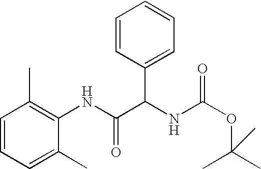

tert-butyl[1-(2,6-dimethylphenylcarbamoyl)-1-phenyl-methyl]-carbamate was isolated (1.61 g, 76%) from BOC-phenyl glycine (1.50 g, 5.97 mmol), N-methylmorpholine (0.72 mL, 6.57 mmol), isobutylchloroformate (0.85 mL, 6.57 mmol), 2,6-dimethylaniline (0.88 mL, 7.16 mmol) and N-methylmorpholine (0.79 mL, 7.16 mmol).

D33 2-Amino-N-(2,6-dimethylphenyl)-2-phenylacetamide

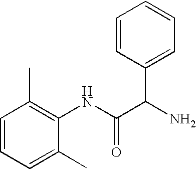

2-Amino-N-(2,6-dimethylphenyl)-2-phenylacetamide was isolated as a white solid (717.2 mg, 67%) from tert-butyl[1-(2,6-dimethylphenylcarbamoyl)-1-phenyl-methyl]-carbamate (1.50 g, 4.24 mmol).

2-Nitro-4-ethoxy-phenylisothiocyanate

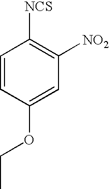

Made by two different methods.

Method A

To a screw cap vial was added 4-ethoxy-2-nitroaniline (307.0 mg, 1.69 mmol), ethyl acetate (6 mL) and thiophosgene (0.39 mL, 5.06 mmol). The bright orange solution was heated at 75° C. for one hour. The reaction mixture was concentrated and the residue was purified on an SPE tube using Hexanes:Ethyl acetate (98:2 to 80:20) as the eluent to yield the title compound (367.5 mg, 97%).

Method B

To a 50 mL round bottom flask was added 4-ethoxy-2-nitroaniline (50.0 mg, 0.27 mmol), di-2-pyridyl thiocarbonate (DPT) (63.7 mg, 0.27 mmol) and CH₂Cl₂ (3 mL). The reaction was allowed to stir at room temperature for three hours. The TLC of the reaction mixture showed some unreacted aniline so another 0.5 eq. of DPT were added (31.9 mg, 0.135 mmol). After twenty minutes the TLC still showed some unreacted aniline so again another 0.5 eq. of DPT were added (31.9 mg, 0.135 mmol) and the reaction was heated to 50° C. for fifteen minutes. The reaction mixture was put through an SPE tube (CH₂Cl₂) to yield the title compound.

E33.1 N-(2,6-dimethylphenyl)-2-[3-(4-ethoxy-2-nitrophenyl)-thioureido]-2-phenyl acetamide

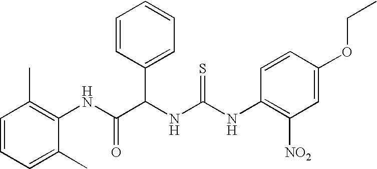

N-(2,6-dimethylphenyl)-2-[3-(4-ethoxy-2-nitrophenyl)-thioureido]-2-phenyl acetamide was isolated as a yellow solid (45.1 mg, 86%) from 2-Amino-N-(2,6-dimethylphenyl)-2-phenylacetamide (28.4 mg, 0.11 mmol) and 2-nitro-4-ethoxyisothiocyanate (30.0 mg, 0.13 mmol).

2-nitro-(N,N-dimethyl)phenyl sulphonamide

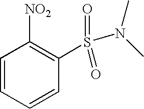

Dimethylamine (2M in THF) (5.64 mL, 11.3 mmol) was added to a screw cap vial and THF was added (2 mL). The solution was cooled to 0° C. and to it was added 2-nitrobenzenesulphonyl chloride (500 mg, 2.26 mmol) in THF (1 mL). The resultant mixture was stirred at room temperature for four hours and then left stirring at 50° C. overnight. The reaction was then allowed to cool to room temperature. The reaction was concentrated and the residue was dissolved in CH₂Cl₂ and was sequentially washed with water, 1N NaOH, water and brine. The organic phase was dried over MgSO₄, filtered and concentrated. The crude product was purified by an SPE tube using Hexanes:EtOAc (90:10 to 20:80) to yield the title compound as a yellow solid (498.4 mg, 96%).

2-amino-(N,N-dimethyl)phenylsulphonamide

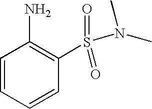

To a 50 mL round bottom flask was added CaCl₂ (30.9 mg, 0.28 mmol) and H₂O (1 mL). To this stirred solution was added Zn-dust (931.2 mg, 14.2 mmol), followed by a solution of 2-nitro-(N,N-dimethyl)phenylsulphonamide (100 mg, 0.43 mmol) in 78% EtOH. The resultant mixture was refluxed for 1.5 hours. The solution was filtered hot over a pad of celite and then concentrated. The residue was taken up in CH₂Cl₂ and sequentially washed with water and brine. The organic phase was dried over MgSO₄, filtered and concentrated. The crude product was purified by an SPE tube using Hexanes: EtOAc (99:1 to 70:30) to yield the title compound as a yellow oil (38.7 mg, 45%).

2-isothiocyanato-(N,N-dimethyl)phenyl sulphonamide

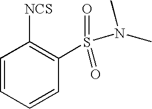

To a screw cap vial was added 2-amino-(N,N-dimethyl)phenylsulphonamide (34.9 mg, 0.17 mmol), DPT (40.5 mg, 0.17 mmol) and CH₂Cl₂ (3 mL). The resultant bright orange solution was stirred at room temperature for 1 hour and forty-five minutes. At this time, DPT was again added (21.0 mg, 0.09 mmol) and after one hour of stirring, a TLC was taken showing the reaction to be 50% complete. DPT was again added (21.0 mg, 0.09 mmol) and the reaction was heated to 50° C. for one hour. The reaction mixture was put through an SPE tube (CH₂Cl₂) to yield the title compound as a yellow oil (34.1 mg, 98%).

E33.2 N-(2,6-dimethylphenyl)-2-[3-(2-N,N-dimethylsulphonamidophenyl)-thioureido]-2-phenyl acetamide

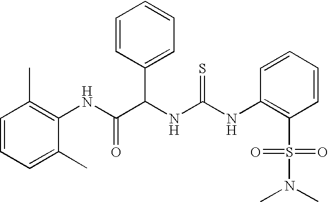

N-(2,6-dimethylphenyl)-2-[3-(2-N,N-dimethylsulphonamidophenyl)-thioureido]-2-phenyl acetamide was isolated as a white solid (49.8 mg, 84%) from 2-Amino-N-(2,6-dimethylphenyl)-2-phenylacetamide (29.7 mg, 0.12 mmol) and 2-N,N-dimethylsulphonamidophenyl isothiocyanate (34.0 mg, 0.14 mmol).

2-nitro-(N-methylpiperazinyl)phenylsulphonamide

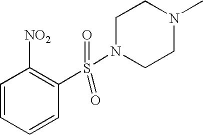

To a screw cap vial was added 1-methylpiperazine (0.25 mL, 2.26 mmol) and diisopropylethylamine (0.39 mL, 2.26 mmol) in CH₂Cl₂ (3 mL). The solution was cooled to 0° C. and a solution of 2-nitrobenzenesulphonylchloride (500 mg, 2.26 mmol) in CH₂Cl₂ (2 mL) was added. The resultant mixture was stirred at room temperature for four hours and then left stirring at 50° C. overnight. The reaction was allowed to cool to room temperature and was diluted with CH₂Cl₂ and the organic phase was sequentially washed with water, 1N NaOH, water and brine. The organic phase was dried over MgSO₄, filtered and concentrated. The crude product was purified by an SPE tube using CH₂Cl₂: MeOH (99:1 to 90:10) to yield the title compound as a yellow oil (624.6 mg, 97%).

2-amino-(N-methylpiperazinyl)phenylsulphonamide

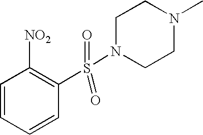

2-amino-(N-methylpiperazinyl)phenylsulphonamide was isolated as a clear oil (134.1 mg, 75%) from CaCl₂ (49.8 mg, 0.45 mmol) and Zn-dust (1.50 g, 23.0 mmol).

2-isothiocyanato-(N-methylpiperazinyl)phenyl sulphonamide

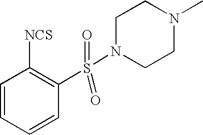

2-isothiocyanato-(N-methylpiperazinyl)phenyl sulphonamide was isolated from 2-amino-(N-methylpiperazinyl)phenylsulphonamide (112.3 mg, 0.44 mmol) and DPT (204.3 mg, 0.88 mmol).

E33.3 N-(2,6-dimethylphenyl)-2-[3-(2-N-methylpiperizinylsulphonamidophenyl)-thioureido]-2-phenyl acetamide

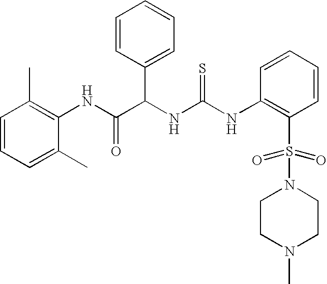

N-(2,6-dimethylphenyl)-2-[3-(2-N-methylpiperizinylsulphonamidophenyl)-thioureido]-2-phenyl acetamide was isolated as a white solid (38.3 mg, 70%) from 2-Amino-N-(2,6-dimethylphenyl)-2-phenylacetamide (26.0 mg, 0.10 mmol) and 2-isothiocyanato-(N-methylpiperazinyl)phenyl sulphonamide (36.5 mg, 0.12 mmol).

4-hydroxy-phenylacetamide

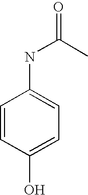

4-aminophenol (5.0 g, 45.8 mmol), water (30 mL) and concentrated HCl (3.8 mL) were added to a round bottom flask. The resultant clear brown solution was stirred at room temperature for five minutes. It was then placed in a 90° C. oil bath and to it was added sodium acetate buffer (6.26 g) in H₂O (18 mL) followed by acetic anhydride (5.2 mL, 55.0 mmol). The resultant clear brown solution was left stirring at 90° C. for two hours. The reaction mixture was cooled down to room temperature and then to 0° C. The reaction mixture was left stirring at 0° C. for forty-five minutes. During this time, a light brown solid precipitated. The solid was filtered, and the water was azeotroped with toluene. The off-white solid was dried under vacuum overnight to yield the title compound (5.33 g, 77%).

4-(2-chloro)ethoxyphenylacetamide

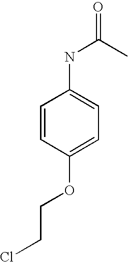

In a three-necked round bottom flask equipped with a stir bar, a reflux condenser and a dropping funnel was added 1-bromo-2-chloroethane a (2.9 mL, 34.7 mmol) and ethanol (10 mL). The mixture was heated to reflux and to it was added a solution of 4-hydroxyphenylacetamide (3.50 g, 23.2 mmol), KOH (2.59 g, 46.3 mmol) in EtOH dropwise. The reaction was left stirring at 85° C. for five hours. The reaction was then allowed to cool to room temperature and the solid was filtered off. The filtrate was concentrated and sequentially washed with 1N NaOH, water and brine. The organic phase was dried over MgSO₄, filtered and concentrated. The crude product was re-crystallized from Hexanes: EtOAc (50:50) to yield the title compound as a white fluffy solid (2.13 g, 43%).

2-nitro-4-(2-chloro)ethoxyphenylacetamide

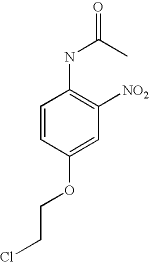

To a round bottom flask equipped with a stirbar was added 4-(2-chloro)ethoxyphenylacetamide (2.12 g, 9.95 mmol) and acetic acid/H₂O mixture (3:2) (37 mL). The reaction was stirred at 50° C. for five minutes and to this clear solution was added HNO₃ (4.7 mL) and the yellow-brown solution was stirred at 70° C. for ten minutes. The mixture was cooled to 0° C. and stirred for two hours at this temperature. The reaction mixture was concentrated and the residue was dissolved in CH₂Cl₂ and washed with 1N NaOH and the aqueous phase was washed twice with CH₂Cl₂. The combined organic phases were sequentially washed with water and brine, dried over MgSO₄, filtered and concentrated. The crude product was purified by CC using Hexanes:EtOAc (90:10, 80:20, 70:30, 60:40) to yield the title compound as a yellow solid (1.92 g, 75%).

2-Nitro-4-(2-(N,N-dimethyl)ethoxy)phenylacetamide

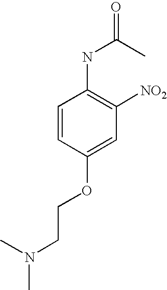

To a solution of 4-(2-chloroethoxy)-2-nitrophenylacetamide (192 mg, 0.74 mmol) in acetonitrile was added KI (167 mg, 1 mmol), K₂CO₃ (150 mg, 1 mmol) and (CH₃)₂NH (2M sol'n in THF, 2 ml, 4 mmol). The mixture was heated to reflux overnight. The reaction mixture was filtered and the filtrate was concentrated in vacuo to give the product as a yellowish brown oil (80 mg, 40% yield).

1-[(2-N,N-dimethylamino)-ethoxy]-3-nitro-4-aniline

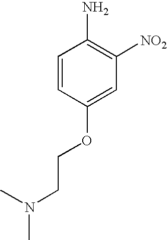

In two separate screw cap vials was added 2-Nitro-4-(2-(N,N-dimethyl)ethoxy)phenylacetamide (821.0 mg, 3.07 mmol) and 20% KOH(aq) (8.0 mL). The reactions were stirred at 110° C. for two hours. The reactions were cooled to room temperature and diluted with brine. The products were extracted with CH₂Cl₂ and the combined organic phases were dried over MgSO₄, filtered and concentrated. The crude product was washed with hexanes and allowed to dry under vacuum to yield the title compound as an orange solid (1.17 g, 85%) (combined).

1-(2-N,N-dimethylamino)-ethoxy-3-nitro-4-phenylisothiocyanate

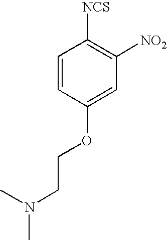

1-(2-N,N-dimethylamino)-ethoxy-3-nitro-4-phenylisothiocyanate was isolated as a dark brown oil (1.11 g, 81%) from 1-[(2-N,N-dimethylamino)-ethoxy]-3-nitro-4-aniline (1.15 g, 5.12 mmol) and DPT (1.31 g, 5.63 mmol).

E33.4 N-(2.6-dimethylphenyl)-2-[3-(4-(2-N,N-dimethylamino)ethoxy-2-nitrophenyl)-thioureido]-2-phenyl acetamide

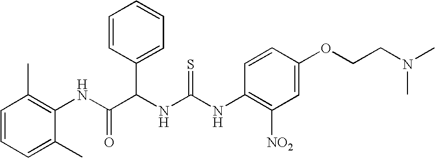

N-(2,6-dimethylphenyl)-2-[3-(2-nitro-4-(2-N,N-dimethylamino)ethoxyphenyl)-thioureido]-2-phenyl acetamide was isolated as a yellow solid (871.6 mg, 82%) from 2-Amino-N-(2,6-dimethylphenyl)-2-phenylacetamide (521.1 mg, 2.05 mmol) and 1-(2-N,N-dimethylamino)-ethoxy-3-nitro-4-phenylisothiocyanate (602.5 mg, 2.25 mmol).

N-(2,6-dimethylphenyl)-2-[3-(2-nitro-4-(2-N,N-dimethylamino)ethoxyphenyl)-thioureido]-2-phenyl acetamide Hydrochloride salt

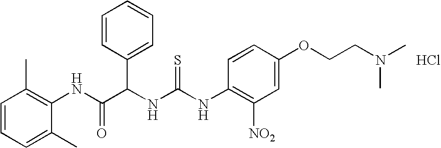

N-(2,6-dimethylphenyl)-2-[3-(2-nitro-4-(2-N,N-dimethylamino)ethoxyphenyl)-thioureido]-2-phenyl acetamide (871.6 mg, 1.67 mmol) was added to a round bottom flask and MeOH (10 mL) was added. To this stirred yellow suspension was added 1N HCl in Et₂O and the mixture was allowed to stir at room temperature for two hours. The reaction was concentrated and the residue was triturated with Et₂O four times. The solid was filtered, washed with Et₂O and dried under vacuum to yield the title compound as a fine yellow solid (891.0 mg, 96%).

1-Chloro-2-nitro-4-(N,N-dimethyl)phenyl sulphonamide

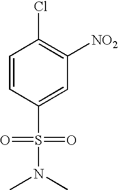

1-chloro-2-nitro-4-(N,N-dimethyl)phenyl sulphonamide was isolated as an off-white solid (104.4 mg, 20%) from 4-chloro-3-nitro benzene sulphonyl chloride (500.0 mg, 1.95 mmol) and dimethylamine (2M in THF) (4.88 mL, 9.76 mmol).

1-amino-2-nitro-4-(N,N-dimethyl)phenylsulphonamide

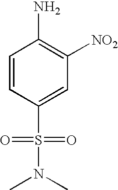

To a screw cap vial was added 1-chloro-2-nitro-4-(N,N-dimethyl)phenyl sulphonamide (100.0 mg, 0.38 mmol) and 2M NH₃/MeOH (5 mL). The reaction was allowed to stir at 55° C. overnight. The reaction TLC showed a lot of starting material so the reaction was placed in a 75° C. oil bath and was allowed to stir for four hours. The reaction was concentrated and the crude product was purified by an SPE tube using CH₂Cl₂ as the eluant to yield the title compound as a yellow solid (14.5 mg, 16%).

2-nitro-4-(N,N-dimethyl)sulphonamido-1-phenylisothiocyanate

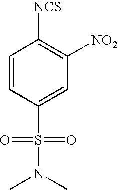

2-nitro-4-(N,N-dimethyl)sulphonamido-1-phenylisothiocyanate was isolated as a light yellow solid (7.1 mg, 41%) from 1-amino-2-nitro-4-(N,N-dimethyl)phenylsulphonamide (13.5 mg, 0.06 mmol) and DPT (12.8 mg, 0.06 mmol).

E33.5 N-(2,6-dimethylphenyl)-2-[3-(4-(2-N,N-dimethylamino)sulphonamido-2-nitro-thioureido]-2-phenyl acetamide

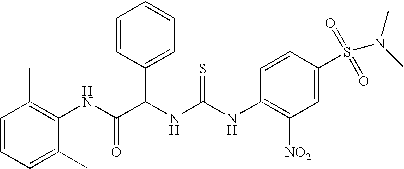

N-(2,6-dimethylphenyl)-2-[3 (4-(2-N,N-dimethylamino)sulphonamido-2-nitro-thioureido]-2-phenyl acetamide was isolated as a light yellow solid (10.0 mg, 95%) from 2-Amino-N-(2,6-dimethylphenyl)-2-phenylacetamide (4.9 mg, 0.02 mmol) and 2-nitro-4-(N,N-dimethyl)sulphonamide-1-phenylisothiocyanate (5.5 mg, 0.02 mmol).

E33.6 N-(2,6-dimethylphenyl)-2-[3-(4-methoxy-2-nitrophenyl)-thioureido]-2-phenyl acetamide

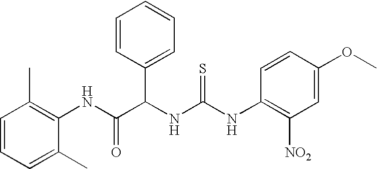

N-(2,6-dimethylphenyl)-2-[3-(4-methoxyphenyl-2-nitro)-thioureido]-2-phenyl acetamide was isolated as a yellow solid (683.3 mg, 94%) from 2-Amino-N-(2,6-dimethylphenyl)-2-phenylacetamide (400.0 mg, 1.57 mmol) and 4-methoxy-2-nitrophenylisothiocyanate (396.9 mg, 1.89 mmol).

E33.7 N-(2.6-dimethylphenyl)-2-[3-(2-trifluoromethylphenyl)-thioureido]-2-phenyl acetamide

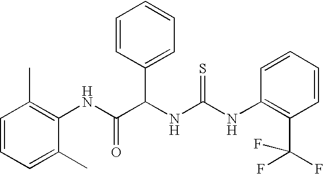

N-(2,6-dimethylphenyl)-2-[3-(2-trifluoromethylphenyl)-thioureido]-2-phenyl acetamide was isolated as a white solid (493.0 mg, 91%) from 2-Amino-N-(2,6-dimethylphenyl)-2-phenylacetamide (300.0 mg, 1.18 mmol) and 2-trifluoromethylphenylisothiocyanate (287.6 mg, 1.42 mmol).

Synthesis of 4-N,N-dimethylaminoethoxy-2-trifluoromethylnitrobenzene

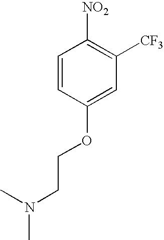

To a stirred mixture of 4-Nitro-3-trifluoromethylphenol (500 mg, 2.4 mmol) and NaOH (193 mg, 4.8 mmol) in water (5 ml) was added xylenes (15 mL) followed by dimethylaminoethylchloride hydrochloride (894.0 mg, 6.0 mmol). The mixture was heated at 125° C. for 24 hrs. The mixture was then cooled to room temperature and the organic layer separated and purified by flash chromatography to give the product as a yellow oil (130 mg, 19%).

Synthesis of 4-[N,N-dimethylaminoethoxy]-2-trifluoromethylaniline

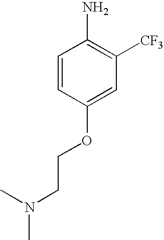

To a suspension of Pd/C (100 mg) in isopropanol (2 ml) was added a solution of 4-N,N-dimethylaminoethoxy-2-trifluoromethylnitrobenzene (130 mg, 0.47 mmol) in isopropanol (2 ml) followed by cyclohexene (2 ml). The mixture was then refluxed under argon for 24 hrs. The mixture was filtered through celite and the solvent removed in vacuo. The crude residue was purified by flash chromatography giving the product as light brown oil (101.0 mg, 86%).

E33.8 N-(2.6-dimethylphenyl)-2-[3-(4-N,N-dimethylaminoethoxy-2-trifluoromethylphenyl)-thioureido]-2-phenyl acetamide

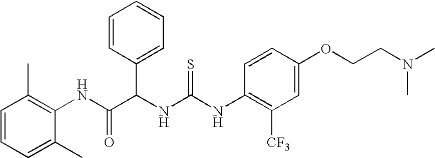

N-(2,6-dimethylphenyl)-2-[3-(4-N,N-dimethylaminoethoxy-2-trifluoromethylphenyl)-thioureido]-2-phenyl acetamide was isolated as an off-white solid, (30 mg, 13%) from 4-[N,N-dimethylaminoethoxy]-2-trifluoromethylaniline (101 mg, 0.41 mmol), thiophosgene (0.94 mL, 1.23 mmol) and 2-amino-N-(2,6-dimethylphenyl)-2-phenylacetamide (203.0 mg, 0.80 mmol).

Synthesis of 4-Bromo-2-trifluoromethylphenylacetamide

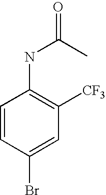

To a solution of 2-amino-5-bromobenzotrifluoride (1.20 g, 5.00 mmol) in toluene (10 ml) at 0° C. was added acetyl chloride (3.9 g, 50 mmol) and pyridine (3.8 g, 48 mmol). The mixture was then refluxed for two hours. After cooling to room temperature, the mixture was diluted with ethylacetate and washed with NaHCO₃ (saturated), brine and the separated organic layer was then dried over anhydrous Na₂SO₄. The solvent was removed in vacuo giving the product as a white solid (1.3 g, 93%).

Synthesis of 4-(4′-pyridyl)-2-trifluoromethylphenylacetamide

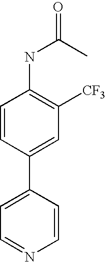

To a mixture of 2-amino-5-bromobenzotrifluoride (200 mg, 0.7 mmol), 4-pyridyl boronic acid (174 mg, 1.4 mmol) in DME (1.5 ml) and Na₂CO₃ (2M aqueous solution, 1.5 ml) was added Pd(PPh₃)₄ (10 mg, 0.009 mmol) and the resulting mixture was heated to 110° C. overnight. The organic layer was separated and then purified by flash chromatography giving the product as a white solid (114.0 mg, 58%).

Synthesis of 4-(N-Methyl-(2,3,6)-tetrahydropyridin-4-yl)-2-trifluoromethylphenylacetamide

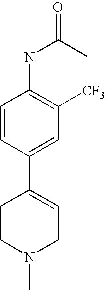

To a solution of 4-(4′-pyridyl)-2-trifluoromethylphenylacetamide (114 mg, 0.4 mmol) in acetonitrile (3 ml) was added methyl iodide (174 mg, 1.2 mmol). The mixture was stirred vigorously for two hours at room temperature after which the mixture was concentrated in vacuo and the crude residue was taken up into MeOH (3 ml) and NaBH₄ (150 mg, 4 mmol) was added and the mixture was stirred for three hours. The solvent was removed and the residue was quenched with water and extracted with CH₂Cl₂. The organic layer was collected, washed with brine, dried over Na₂SO₄ and concentrated in vacuo. Flash chromatography of the crude residue afforded the product as a light yellow oil (98.0 mg, 82%)

Synthesis of 4-(N-Methyl-(2,3,6)-tetrahydropyridin-4-yl)-2-trifluoromethylaniline

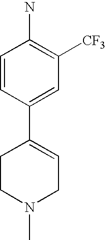

4-(N-Methyl-(2,3,6)-tetrahydropyridin-4-yl)-2-trifluoromethyl phenylacetamide (98 mg, 0.33 mmol) was added to 20% KOH (aq) (2 mL) and was heated at 100° C. for two hours. The mixture was then cooled and extracted four times with CH₂Cl₂. The extract was then dried over Na₂SO₄ (anhydrous) and the solvent was evaporated to provide the title compound as a white solid (65.0 mg, 77%).

E33.9 N-(2.6-dimethylphenyl)-2-[3-(N-Methyl-(2,3,6)-tetrahydropyridin-4-yl-2-trifluoromethylphenyl)-thioureido]-2-phenyl acetamide

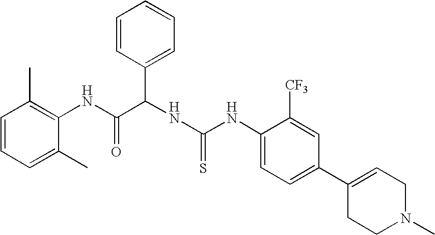

N-(2,6-di methyl phenyl)-2-[3-(N-Methyl-(2,3,6)-tetrahydropiperidin-4-yl-2-trifluoromethylphenyl)-thioureido]-2-phenyl acetamide was isolated as an off-white solid, 60 mg, 43%) from 4-(N-Methyltetrahydropiperidin-4-yl)-2-trifluoromethylaniline (65 mg, 0.25 mmol) and 2-amino-N-(2,6-dimethylphenyl)-2-phenyl acetamide (127.0 mg, 0.5 mmol).

E33.10 N-(2.6-dimethylphenyl)-2-[3-(4-methyl-2-nitrophenyl)-thioureido]-2-phenyl acetamide

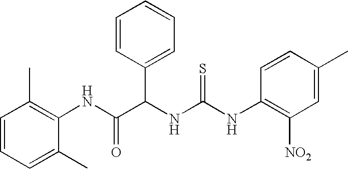

N-(2,6-dimethylphenyl)-2-[3-(4-methyl-2-nitrophenyl)-thioureido]-2-phenyl acetamide was isolated as a yellow solid (10 mg, 14%) from 4-methyl-2-nitrophenylisothiocyanate (32.5 mg, 0.16 mmol) and N-(2,6-dimethylphenyl) phenylglycinamide (50 mg, 0.197 mmol).

E33.11 N-(2.6-dimethylphenyl)-2-[3-(2-nitrophenyl)-thioureido]-2-phenyl acetamide

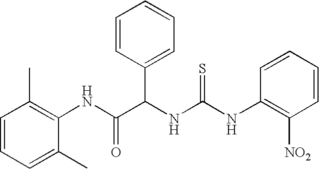

N-(2,6-dimethylphenyl)-2-[3-(2-nitrophenyl)-thioureido]-2-phenyl acetamide was isolated as a yellow solid, (13 mg, 60%) from 2-nitrophenylisothiocyanate (13.5 mg, 0.075 mmol) and N-(2,6-dimethylphenyl)phenylglycinamide (13 mg, 0.05 mmol).

### Example 34

C34 1-tert butylcarbamoyl-1-[N-(2-isopropylphenyl)-cyclohexane carboxamide

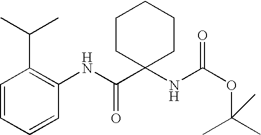

1-tert butylcarbamoyl-1-[N-(2-isopropylphenyl)-cyclohexane carboxamide was isolated as a white solid (295.5 mg, 63%) from 1-(Tert-butoxycarbonylamino) cyclohexanecarboxylic acid (200.0 mg, 0.82 mmol), 2-isopropylaniline (0.13 mL, 0.90 mmol), DECP (0.19 mL, 1.23 mmol) and 1-methylimidazole (0.13 mL, 1.64 mmol).

D34 1-Amino-N-(2-isopropylphenyl)cyclohexane carboxamide

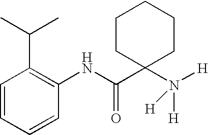

1-Amino-N-(2-isopropylphenyl)cyclohexane carboxamide was isolated as a pale yellow oil (101.6 mg, 68%) from 1-tert butylcarbamoyl-1-[N-(2-isopropylphenyl)-cyclohexane carboxamide (182.5 mg, 0.51 mmol).

E34.1 [1-(2-nitrophenyl)-thioureido N-(2-isopropylphenyl]-cyclohexane carboxamide

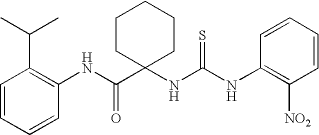

[1-(2-nitrophenyl)-thioureido N-(2-isopropylphenyl]-cyclohexane carboxamide was isolated as a bright yellow solid (14.4 mg, 20%) from 1-Amino-N-(2-isopropylphenyl)cyclohexane carboxamide (38.7 mg, 0.13 mmol), 2-nitrophenylisothiocyanate (27.0 mg, 0.15 mmol) and triethylamine (0.023 mL, 0.16 mmol).

### Example 35

C35 1-tert butyl-carbamoyl-1-[N-(2-phenylphenyl)]-cyclohexane carboxamide

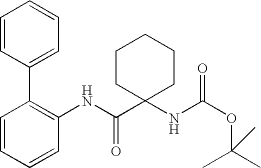

1-tert butyl-carbamoyl-1-[N-(2-phenylphenyl)]-cyclohexane carboxamide was isolated as a salmon-coloured solid (323.4 mg, 51%) from 1-(Tert-butoxycarbonylamino) cyclohexanecarboxylic acid (200 mg, 0.82 mmol), 2-phenylaniline (153.0 mg, 0.90 mmol), 1-methylimidazole (0.13 mL, 1.64 mmol) and DECP (0.19 mL, 1.23 mmol).

D35 1-Amino-N-(2-phenylphenyl)cyclohexane carboxamide

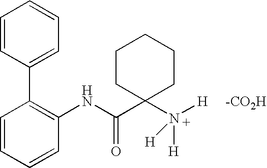

1-Amino-N-(2-phenylphenyl)cyclohexane carboxamide was isolated as a pale pink solid (89.2 mg, 65%) from 1-tert butyl-carbamoyl-1-[N-(2-phenylphenyl)]-cyclohexane carboxamide (158.5 mg, 0.40 mmol).

E35.1 [1-(4-methoxycarbonylphenyl)-thioureido N-(2-phenylphenyl)]-cyclohexane carboxamide

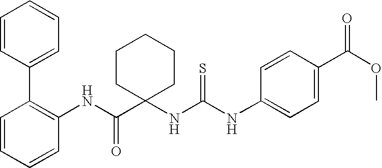

[1-(4-methoxycarbonyl phenyl)-thioureido N-(2-phenyl phenyl)]-cyclohexane carboxamide was isolated as a yellow solid (2.2 mg, 4%) from 1-Amino-N-(2-phenylphenyl)cyclohexane carboxamide (40.0 mg, 0.12 mmol), 4-methoxycarbonylisothiocyanate (27.0 mg, 0.14 mmol) and triethylamine (0.03 mL, 0.24 mmol).

E35.2 [1-(2-nitrophenyl)-thioureido N-(2-phenylphenyl)]-cyclohexane carboxamide

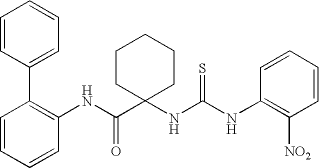

[1-(2-nitrophenyl)-thioureido N-(2-phenylphenyl)]-cyclohexane carboxamide was isolated as a yellow solid (11.8 mg, 21%) from 1-Amino-N-(2-biphenyl)cyclohexane carboxamide (40.0 mg, 0.12 mmol) and 2-nitrophenylisothiocyanate (25.0 mg, 0.14 mmol).

### Example 36

C36 1-tert butyl-carbamoyl-1-[N-(4-methylphenyl)]-cyclohexane carboxamide

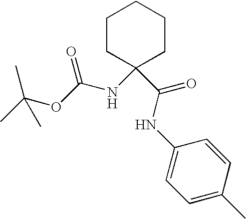

1-tert butyl-carbamoyl-1-[N-(4-methylphenyl)]-cyclohexane carboxamide was isolated as a white solid (36.9 mg, 27%) from 1-(tert-butoxycarbonylamino)cyclohexanecarboxylic acid (100.0 mg, 0.41 mmol), 1-methylimidazole (0.065 mL, 0.82 mmol), DECP (0.094 mL, 0.62 mmol) and 4-methylaniline (48.0 mg, 0.45 mmol).

D36 1-Amino-N-(4-methylphenyl)cyclohexane carboxamide

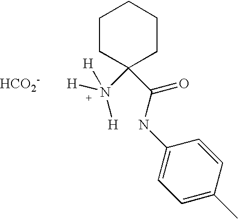

1-Amino-N-(4-methylphenyl)cyclohexane carboxamide was isolated as a white solid (26.6 mg, 88%) from 1-tert butyl-carbamoyl-1-[N-(4-methylphenyl)]-cyclohexane carboxamide (36.0 mg, 0.11 mmol).

E36.1 [1-(4-methoxycarbonylphenyl)-thioureido N-(4-methyl)phenyl]-cyclohexane carboxamide

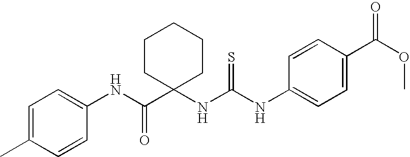

[1-(4-methoxycarbonyl phenyl)-thioureido N-(4-methyl)phenyl]-cyclohexane carboxamide was isolated as a yellow solid (24.1 mg, 66%) from 1-Amino-N-(4-methylphenyl)cyclohexane carboxamide (24.0 mg, 0.09 mmol), 4-methoxycarbonylphenylisothiocyanate (20.0 mg, 0.10 mmol) and triethylamine (0.016 mL, 0.11 mmol).

### Example 37

C37 tert-butyl[1-(4-methylphenylcarbamoyl)-1-phenyl-methyl]-carbamate

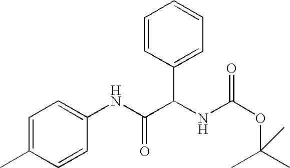

tert-butyl[1-(4-methylphenylcarbamoyl)-1-phenyl-methyl]-carbamate was isolated as a white solid (1.88 g, 139%) from [(tert-butoxycarbonyl)amino](phenyl)acetic acid (1.0 g, 3.98 mmol), N-methylmorpholine (0.48 mL, 4.38 mmol), isobutylchloroformate (0.57 mL, 4.38 mmol) and 4-methylaniline (0.51 g, 4.78 mmol) and N-methylmorpholine (0.52 mL, 4.78 mmol).

D37 2-Amino-N-(4-methylphenyl)-2-phenylacetamide

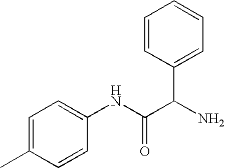

To a 50 mL round bottom flask equipped with a stirring bar was added tert-butyl [1-(4-methylphenylcarbamoyl)-1-phenyl-methyl]-carbamate (1.88 g, 5.52 mmol) and formic acid (15 mL). The reaction was stirred at 50° C. for one and a half hours after which it was cooled to room temperature. The formic acid was removed in vacuo and the resulting oil was taken up into EtOAc (75 mL) and water (75 mL). 1N NaOH was added until the pH was 8-9 and the aqueous layer was extracted with EtOAc two additional times. The combined organic layers were washed with brine, dried over MgSO₄, filtered and concentrated. The crude product was purified by CC (Hexanes:EtOAc 60:40 to CH₂Cl₂:MeOH 95:5 to yield the title compound as an off-white solid (874.2 mg, 91%).

E37.1 N-(4-methylphenyl)-2-[3-(4-methoxycarbonylphenyl)-thioureido]-2-phenyl acetamide

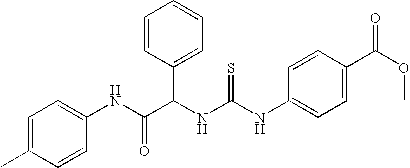

N-(4-methylphenyl)-2-[3-(4-methoxycarbonylphenyl)-thioureido]-2-phenyl acetamide was isolated as a white solid (38.7 mg, 74%) from 2-Amino-N-(4-methylphenyl)-2-phenylacetamide (29.8 mg, 0.12 mmol) and 4-methoxycarbonylphenylisothiocyanate (36.0 mg, 0.19 mmol).

E37.2 N-(4-methylphenyl)-2-[3-(1-naphthyl)-thioureido]-2-phenyl acetamide

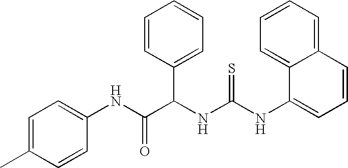

N-(4-methylphenyl)-2-[3-(1-naphthyl)-thioureido]-2-phenyl acetamide was isolated as a white solid (37.4 mg, 70%) from 2-Amino-N-(4-methylphenyl)-2-phenylacetamide (30.3 mg, 0.13 mmol) and 1-naphthylisothiocyanate (35.0 mg, 0.19 mmol).

E37.3 N-(4-methylphenyl)-2-[3-(2-nitrophenyl)-thioureido]-2-phenyl acetamide

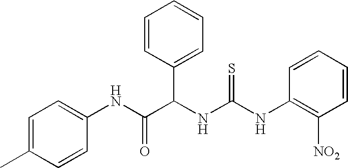

N-(4-methylphenyl)-2-[3-(2-nitrophenyl)-thioureido]-2-phenyl acetamide was isolated as a yellow solid (28.2 mg, 54%) from 2-Amino-N-(4-methylphenyl)-2-phenylacetamide (29.9 mg, 0.12 mmol) and 2-nitrophenylisothiocyanate (34.0 mg, 0.19 mmol).

### Example 38

C38 tert-butyl[1-(phenylcarbamoyl)-1-phenyl-methyl]-carbamate

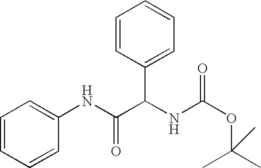

tert-butyl[1-(phenylcarbamoyl)-1-phenyl-methyl]-carbamate was isolated as a white solid (1.42 g, 110%) from [(tert-butoxycarbonyl)amino](phenyl)acetic acid (1.0 g, 3.98 mmol), N-methylmorpholine (0.48 mL, 4.38 mmol), isobutylchloroformate (0.57 mL, 4.38 mmol), aniline (0.44 mL, 4.78 mmol) and N-methylmorpholine (0.52 mL, 4.78 mmol).

D38 2-Amino-N-(phenyl)-2-phenylacetamide

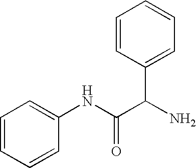

2-Amino-N-(phenyl)-2-phenylacetamide was isolated as a white solid (754.5 mg, 84%) from teri-butyl[1-(phenylcarbamoyl)-1-phenyl-methyl]-carbamate (1.42 g, 4.36 mmol).

E38.1 N-(phenyl)-2-[3-(2-nitrophenyl)-thioureido]-2-phenyl acetamide

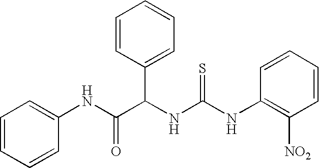

N-(phenyl)-2-[3-(2-nitrophenyl)-thioureido]-2-phenyl acetamide was isolated as a yellow solid (44.0 mg, 80%) from 2-Amino-N-(phenyl)-2-phenylacetamide (30.8 mg, 0.14 mmol) and 2-nitrophenylisothiocyanate (37.0 mg, 0.20 mmol).

E38.2 N-(phenyl)-2-[3-(1-naphthyl)-thioureido]-2-phenyl acetamide

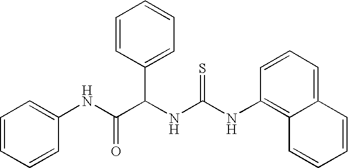

N-(phenyl)-2-[3-(1-naphthyl)-thioureido]-2-phenyl acetamide was isolated as a white solid (48.2 mg, 79%) from 2-Amino-N-(phenyl)-2-phenylacetamide (33.8 mg, 0.15 mmol) and 1-naphthylisothiocyanate (41.0 mg, 0.22 mmol).

E38.3 N-(phenyl)-2-[3-(4-methoxycarbonylphenyl)-thioureido]-2-phenyl acetamide

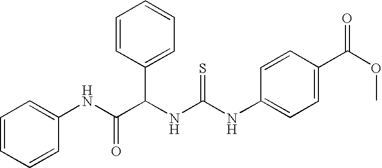

N-(phenyl)-2-[3-(4-methoxycarbonylphenyl)-thioureido]-2-phenyl acetamide was isolated as an off-white solid (35.7 mg, 65%) from 2-Amino-N-(phenyl)-2-phenylacetamide (30.3 mg, 0.13 mmol) and 4-methoxycarbonylphenylisothiocyanate (39.0 mg, 0.20 mmol).

E38.4

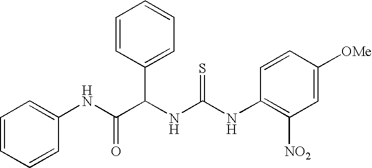

### Example 39

C39 tert-butyl[1-(3-methylphenylcarbamoyl)-1-phenyl-methyl]-carbamate

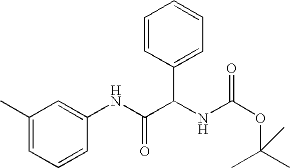

tert-butyl[1-(3-methylphenylcarbamoyl)-1-phenyl-methyl]-carbamate was isolated as an off-white solid (1.46 g, 108%) from [(tert-butoxycarbonyl)amino](phenyl)acetic acid (1.0 g, 3.98 mmol), N-methylmorpholine (0.48 mL, 4.38 mmol), isobutylchloroformate (0.57 mL, 4.38 mmol), 3-methylaniline (0.52 mL, 4.78 mmol) and N-methylmorpholine (0.52 mL, 4.78 mmol).

D39 2-Amino-N-(3-methylphenyl)-2-phenylacetamide

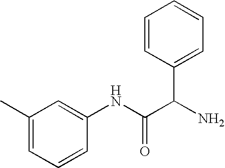

2-Amino-N-(3-methylphenyl)-2-phenylacetamide was isolated as an off-white solid (917.1 mg, 96%) from tert-butyl[1-(3-methylphenylcarbamoyl)-1-phenyl-methyl]-carbamate (1.46 g, 4.29 mmol).

E39.1 N-(3-methylphenyl)-2-[3-(4-methoxycarbonylphenyl)-thioureido]-2-phenyl acetamide

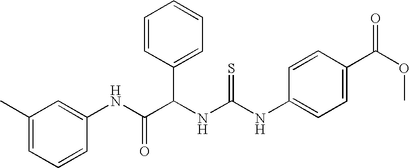

N-(3-methylphenyl)-2-[3-(4-methoxycarbonylphenyl)-thioureido]-2-phenyl acetamide was isolated as a white solid (38.9 mg, 73%) from 2-Amino-N-(3-methylphenyl)-2-phenylacetamide (30.6 mg, 0.13 mmol) and 4-methoxycarbonylphenylisothiocyanate (36.9 mg, 0.19 mmol).

E39.2 N-(3-methylphenyl)-2-[3-(1-naphthyl)-thioureido]-2-phenyl acetamide

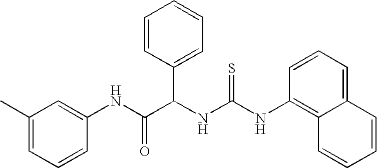

N-(3-methylphenyl)-2-[3-(1-naphthyl)-thioureido]-2-phenyl acetamide was isolated as a white solid (24.8 mg, 48%) from 2-Amino-N-(3-methylphenyl)-2-phenylacetamide (29.5 mg, 0.12 mmol) and 1-naphthylisothiocyanate (34.1 mg, 0.18 mmol).

E39.3 N-(3-methylphenyl)-2-[3-(2-nitrophenyl)-thioureido]-2-phenyl acetamide

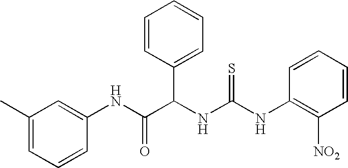

N-(3-methyl phenyl)-2-[3-(2-nitrophenyl)-thioureido]-2-phenyl acetamide was isolated as a yellow solid (33.2 mg, 58%) from 2-Amino-N-(3-methylphenyl)-2-phenylacetamide (32.5 mg, 0.14 mmol) and 2-nitrophenylisothiocyanate (36.5 mg, 0.20 mmol).

E39.4 N-(3-methylphenyl)-2-[3-(2-nitro-4-methoxyphenyl)-thioureido]-2-phenyl acetamide

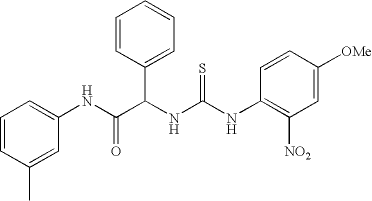

### Example 40

C40 1-tert butyl-carbamoyl-1-[N-(2-methoxyphenyl)]-cyclohexane carboxamide

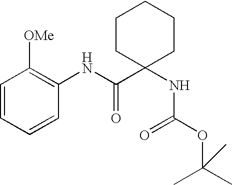

1-tert butyl-carbamoyl-1-[N-(2-methoxyphenyl)]-cyclohexane carboxamide was isolated as a beige solid (234.5 mg, 82%) from 1-(Tert-butoxycarbonylamino) cyclohexanecarboxylic acid (200.0 mg, 0.82 mmol), 2-methoxyaniline (0.10 mL, 0.90 mmol), 1-methylimidazole (0.13 mL, 1.64 mmol) and DECP (0.19 mL, 1.23 mmol).

D40 1-Amino-N-(2-methoxyphenyl)cyclohexane carboxamide

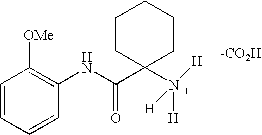

1-Amino-N-(2-methoxyphenyl)cyclohexane carboxamide was isolated as a brown sticky solid (124.4 mg, 65%) from 1-tert butyl-carbamoyl-1-[N-(2-methoxyphenyl)]-cyclohexane carboxamide (227.5 mg, 0.65 mmol).

E40.1 [1-(4-methoxycarbonylphenyl)-thioureido N-(2-methoxyphenyl)]-cyclohexane carboxamide

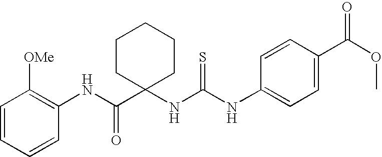

[1-(4-methoxycarbonyl phenyl)-thioureido N-(2-methoxyphenyl]-cyclohexane carboxamide was isolated as a beige solid (8.1 mg, 11%) from 1-Amino-N-(2-methoxyphenyl)cyclohexane carboxamide (50.0 mg, 0.17 mmol), 4-methoxycarbonylisothiocyanate (39.0 mg, 0.20 mmol) and triethylamine (0.05 mL, 0.34 mmol).

### Example 41

C41 1-tert butyl-carbamoyl-1-[N-(2-ethylphenyl)]-cyclohexane carboxamide

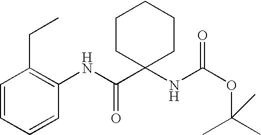

1-tert butyl-carbamoyl-1-[N-(2-ethylphenyl)]-cyclohexane carboxamide was isolated as an off-white solid (78.9 mg, 28%) from 1-(Tert-butoxycarbonylamino) cyclohexanecarboxylic acid (200.0 mg, 0.82 mmol), 1-methylimidazole (0.13 mL, 1.64 mmol), DECP (0.19 mL, 1.23 mmol) and 2-ethyl aniline (0.11 mL, 0.90 mmol).

D41 1-Amino-N-(2-ethylphenyl)cyclohexane carboxamide

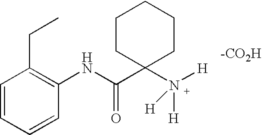

1-Amino-N-(2-ethylphenyl)cyclohexane carboxamide was isolated as a white solid (35.1 mg, 56%) from 1-tert butyl-carbamoyl-1-[N-(2-ethylphenyl)]-cyclohexane carboxamide (73.9 mg, 0.21 mmol).

E41.1 [1-(2-nitrophenyl)-thioureido N-(2-ethylphenyl)]-cyclohexane carboxamide

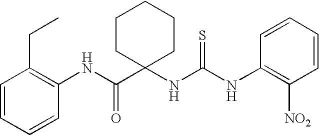

[1-(2-nitrophenyl)-thioureido N-(2-ethylphenyl)]-cyclohexane carboxamide was isolated as a yellow solid (7.0 mg, 14%) from 1-Amino-N-(2-ethylphenyl)cyclohexane carboxamide (34.0 mg, 0.12 mmol), 2-nitrophenylisothiocyanate (25.0 mg, 0.14 mmol) and triethylamine (0.032 mL, 0.23 mmol).

### Example 42

D42 (R)-2-amino-N-(2,6-dimethylphenyl)-2-cyclohexyl acetamide

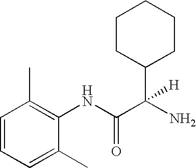

Boc-D-□-cyclohexylglycine (250.0 mg, 0.971 mmol) was added to THF (2 mL) at 0° C. N-methylmorpholine (100.0 mg, 1.068 mmol) and isobutylchloroformate (145.9 mg, 1.068 mmol) were added and the reaction was allowed to stir at 0° C. for two hours. 2,6-dimethylaniline (141.2 mg, 1.165 mmol) was added in THF (2 mL) and stirred at room temperature for one hour. The THF was evaporated and CH₂Cl₂ was added and washed sequentially with (sat.) NaHCO₃, 1M NaHSO₄, water and brine. Formic acid was added to the product and the reaction was allowed to stir at 50° C. for one hour. The formic acid was evaporated and the crude product was purified by column chromatography (50% EtOAc in Hexane) to yield the title compound (31.0 mg, 12%).

E42.1 (R)-N-(2,6-dimethylphenyl)-2-[3-(4-methoxy-2-nitrophenyl)-thioureido]-2-cyclohexyl acetamide

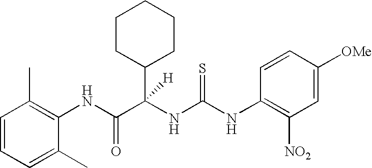

N-(2,6-dimethylphenyl)-2-[3-(4-methoxy-2-nitrophenyl)-thioureido]-2-cyclohexyl acetamide was isolated (14.6 mg, 54%) from (R)-2-amino-N-(2,6-dimethylphenyl)-2-cyclohexyl acetamide (15 mg, 0.057 mmol) and 4-methoxy-2-nitrophenylisothiocyanate (18 mg, 0.086 mmol).

E42.2 (R)-N-(2.6-dimethylphenyl)-2-[3-(2-trifluoromethylphenyl)-thioureido]-2-cyclohexyl acetamide

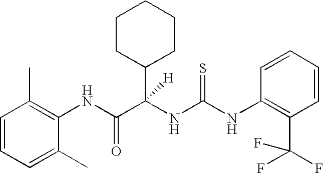

(R)-N-(2,6-dimethylphenyl)-2-[3-(2-trifluoromethylphenyl)-thioureido]-2-cyclohexyl acetamide was isolated as a white powder (12.0 mg, 45%) from (R)-2-amino-N-(2,6-dimethylphenyl)-2-cyclohexyl acetamide (15.0 mg, 0.057 mmol) and 2-trifluoromethylphenylisothiocyanate (17.5 mg, 0.086 mmol).

### Example 43

E43.1 N-(2-isopropylphenyl)-2-[3-(2-nitro-4-methoxyphenyl)-thioureido]-2-phenyl acetamide

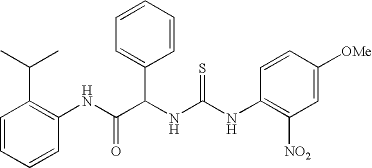

N-(2-isopropylphenyl)-2-[3-(2-nitro-4-methoxyphenyl)-thioureido]-2-phenyl acetamide was isolated (23.0 mg, 43%) from 2-amino-N-(2-isopropylphenyl)-2-phenylacetamide (30.0 mg, 0.111 mmol) and 2-nitro-4-methoxyphenylisothiocyanate (34.9 mg, 0.166 mmol).

E43.2 N-(2-isopropylphenyl)-2-[3-(2-nitrophenyl)-thioureido]-2-phenyl acetamide

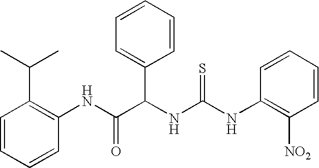

N-(2-isopropylphenyl)-2-[3-(2-nitrophenyl)-thioureido]-2-phenyl acetamide was isolated (20.0 mg, 40%) from 2-amino-N-(2-isopropylphenyl)-2-phenylacetamide (30.0 mg, 0.111 mmol) and 2-nitrophenylisothiocyanate (30.0 mg, 0.166 mmol).

E43.3 N-(2-isopropylphenyl)-2-[3-(2-trifluoromethylphenyl)-thioureido]-2-phenyl acetamide

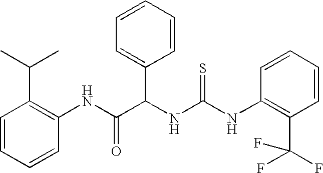

N-(2-isopropylphenyl)-2-[3-(2-trifluoromethylphenyl)-thioureido]-2-phenyl acetamide was isolated (17.0 mg, 33%) from 2-amino-N-(2-isopropylphenyl)-2-phenylacetamide (30.0 mg, 0.111 mmol) and 2-trifluoromethylphenylisothiocyanate (33.7 mg, 0.166 mmol).

E43.4 N-(2-isopropyl)-2-[3-(2-nitrophenyl)-thioureido]-2-phenyl acetamide

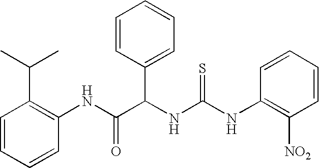

N-(2-isopropyl)-2-[3-(2-nitrophenyl)-thioureido]-2-phenyl acetamide was isolated (42.0 mg, 125%) from 2-amino-N-(2-isopropyl)-2-phenylacetamide (20 mg, 0.075 mmol) and 2-nitrophenyl isothiocyanate (20.4 mg, 0.113 mmol).

### Example 44

E44.1 N-(2-phenylphenyl)-2-[3-(2-nitrophenyl)-thioureido]-2-phenyl acetamide

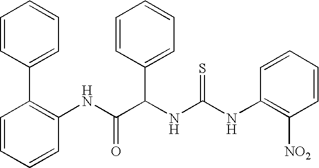

N-(2-phenylphenyl)-2-[3-(2-nitrophenyl)-thioureido]-2-phenyl acetamide was isolated (21.0 mg, 44%) from 2-amino-N-(2-biphenyl)-2-phenylacetamide (30.0 mg, 0.099 mmol) and 2-nitrophenylisothiocyanate (28.0 mg, 0.149 mmol).

### Example 45

C45 tert-butyl[1-(2-naphthyl carbamoyl)-1-phenyl-methyl]carbamate

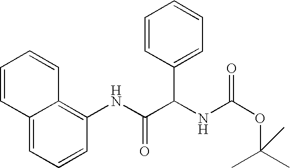

tert-butyl[1-(2-naphthyl carbamoyl)-1-phenyl-methyl] carbamate was isolated (1.45 g, 97%) from tert-butoxy carbonyl phenyl glycine (1.0 g, 3.98 mmol), isobutyl chloroformate (569.4 ul, 4.78 mmol), N-methylmorpholine (482 ul, 4.38 mmol) and 1-aminonaphthalene (686.8 mg, 4.78 mmol).

D45 2-Amino-N-(2-naphthyl)-2-phenylacetamide

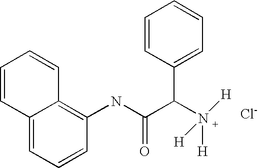

2-Amino-N-(2-naphthyl)-2-phenylacetamide was isolated (640.0 mg, 79%) from tert-butyl[1-(2-naphthyl carbamoyl)-1-phenyl-methyl]carbamate (941.1 mg, 2.5 mmol), formic acid (18 mL) and then treated with 1M HCl (3 mL).

E45.1 N-(2-naphthyl)-2-[3-(2-nitrophenyl)-thioureido]-2-phenyl acetamide

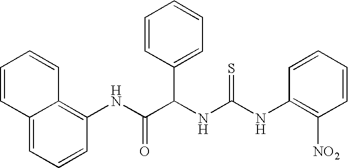

N-(2-naphthyl)-2-[3-(2-nitrophenyl)-thioureido]-2-phenyl acetamide (5.1 mg, 12%) was isolated as a yellow solid from 2-Amino-N-(2-naphthyl)-2-phenylacetamide hydrochloride salt (30.0 mg, 0.096 mmol) and 2-nitrophenyl thioisocyanate (25.9 mg, 0.144 mmol).

E45.1 N-(2-(N-methylpiperizinyl)phenyl)-2-[3-(2-trifluoromethylphenyl)-thioureido]-2-phenyl acetamide

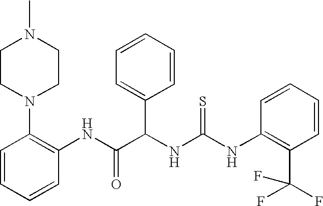

N-(2-(N-methylpiperizinyl)phenyl)-2-[3-(2-trifluoromethylphenyl)-thioureido]-2-phenyl acetamide was isolated (10.0 mg, 21%) from 2-amino-N-(2-(N-methylpiperizinyl)phenyl)-2-phenylacetamide (30.0 mg, 0.092 mmol) and 2-trifluoromethylphenylisothiocyanate (28.2 mg, 0.138 mmol).

E45.2 N-(2-N-methylpiperizinyl)-2-[3-(2-nitrophenyl)-thioureido]-2-phenyl acetamide

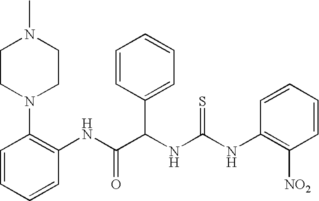

N-(2-N-methylpiperizinyl)-2-[3-(2-nitrophenyl)-thioureido]-2-phenyl acetamide was isolated (55.0 mg, 50%) from 2-Amino-N-(2-N-methylpiperizinyl)-2-phenyl acetamide (70 mg, 0.216 mmol) and 2-nitrophenyl isothiocyanate (58.4 mg, 0.324 mmol).

### Example 46

D46 2-Amino-N-(2-methylphenyl)-2-(3,4-difluorophenyl)acetamide

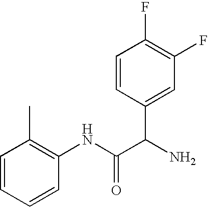

2-Amino-N-(2-methylphenyl)-2-(3,4-difluorophenyl)acetamide (129.9 mg, 54%) was isolated from 3,4-difluoro-N-Boc-phenyl glycine (250.0 mg, 0.870 mmol), N-methylmorpholine (0.097 mL, 0.880 mmol), isobutylchloroformate (0.13 mL, 0.957 mmol), o-toluidine (112.0 mg, 1.044 mmol) and N-methylmorpholine (0.10 mL, 0.910 mmol) and formic acid.

E46.1 N-(2-methylphenyl)-2-[3-(2-nitro-4-methoxyphenyl)-thioureido]-2-(3.4-difluoro)phenyl acetamide

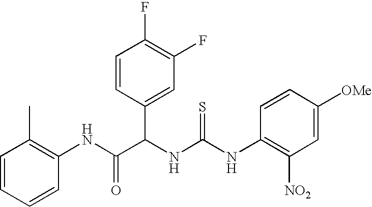

N-(2-methylphenyl)-2-[3-(2-nitro-4-methoxyphenyl)-thioureido]-2-(3,4-difluorophenyl acetamide was isolated as a yellow solid (30.6 mg, 87%) from 2-amino-N-(2-methylphenyl)-2-(3,4-difluoro)phenylacetamide (20.0 mg, 0.072 mmol) and 2-nitro-4-methoxyphenylisothiocyanate (22.7 mg, 0.108 mmol).

E46.2 N-(2-methylphenyl)-2-[3-(2-trifluoromethylphenyl)-thioureido]-2-(3,4-difluoro)phenyl acetamide

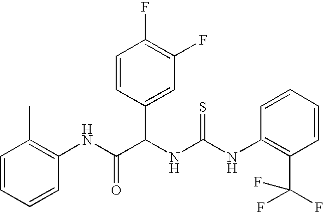

N-(2-methylphenyl)-2-[3-(2-trifluoromethylphenyl)-thioureido]-2-(3,4-difluoro)phenyl acetamide was isolated as a white powder (26.8 mg, 78%) from 2-amino-N-(2-methylphenyl)-2-(3,4-difluoro)phenylacetamide (20.0 mg, 0.072 mmol) and 2-trifluoromethylphenylisothiocyanate (22.0 mg, 0.108 mmol).

E46.3 N-(2-methylphenyl)-2-[3-(2-nitrophenyl)-thioureido]-2-(3.4-difluoro)phenyl acetamide

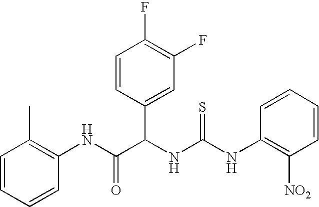

N-(2-methylphenyl)-2-[3-(2-nitrophenyl)-thioureido]-2-(3,4-difluoro)phenyl acetamide was isolated as a light yellow powder (30.2 mg, 61%) from 2-amino-N-(2-methylphenyl)-2-(3,4-difluoro)phenylacetamide (30.0 mg, 0.108 mmol) and 2-nitrophenylisothiocyanate (30.6 mg, 0.162 mmol).

### Example 47

D47 2-Amino-N-(2-methylphenyl)-2-(3-trifluoromethylphenyl)acetamide

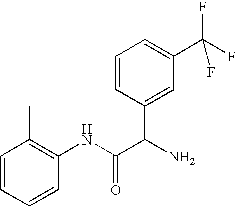

2-Amino-N-(2-methylphenyl)-2-(3-trifluoromethylphenyl)acetamide was isolated from 3-trifluoromethyl-N-Boc phenylglycine (250.0 mg, 0.782 mmol), N-methylmorpholine (0.087 mL, 0.861 mmol), isobutylchloroformate (0.20 mL, 0.861 mmol), o-toluidine (100.5 mg, 0.938 mmol) and N-methylmorpholine (0.095 mL, 0.872 mmol) and formic acid.

E47.1 N-(2-(2-methylphenyl)-2-[3-(2-nitrophenyl)-thioureido]-2-(3-trifluoromethyl)phenyl acetamide

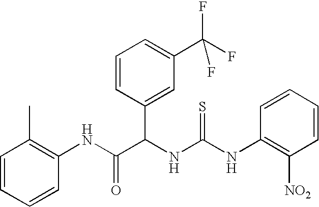

N-(2-(2-methylphenyl)-2-[3-(2-nitrophenyl)-thioureido]-2-(3-trifluoromethyl)phenyl acetamide was isolated as a light yellow powder (18.4 mg, 39%) from 2-amino-N-(2-methylphenyl)-2-(3-trifluoromethyl)phenylacetamide (30.0 mg, 0.097 mmol) and 2-nitrophenylisothiocyanate (27.6 mg, 0.145 mmol).

### Example 48

B48 N-tert-butoxycarbonyl-2-(3-thiophenyl)glycine

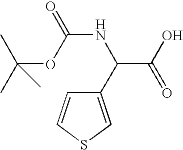

N-tert-butoxycarbonyl-2-(3-thiophenyl)glycine was isolated as a white solid (560.0 mg, 68%) from 2-(3-thiophenyl)glycine (500.0 mg, 3.2 mmol), BOC₂₀ (1.04 g, 4.8 mmol), Et₃N (647.0 mg, 6.4 mmol) and NaOH (128.0 mg, 3.2 mmol).

C48 tert butyl[1-(2-methylphenyl carbamoyl)-1-(3-thiophenyl)methyl]carbamate

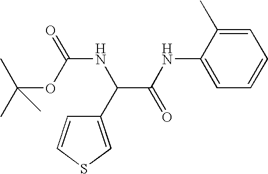

tert butyl[1-(2-methylphenyl carbamoyl)-1-(3-thiophenyl)methyl] carbamate was isolated (611.0 mg, 88%) from N-tert-butoxycarbonyl-2-(3-thiophenyl)glycine (530.0 mg, 2.0 mmol), N-methylmorpholine (230.0 mg, 2.3 mmol), isobutylchloroformate (314.0 mg, 2.3 mmol) and o-toluidine (246.0 mg, 2.3 mmol).

D48 2-amino-N-(2-methylphenyl)-2-(3-thiophenyl)acetamide

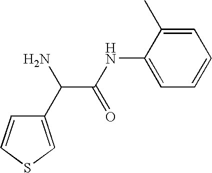

2-amino-N-(2-methylphenyl)-2-(3-thiophenyl)acetamide was isolated as a white solid (40.0 mg, 28%) from tert butyl[1-(2-methylphenyl carbamoyl)-1-(3-thiophenyl)methyl] carbamate (200.0 mg, 0.578 mmol).

E48.1 N-(2-methylphenyl)-2-[3-(4-ethoxy-2-nitrophenyl)-thioureido]-2-[3-thienyl]acetamide

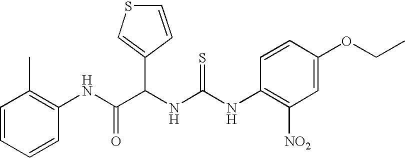

N-(2-methylphenyl)-2-[3-(4-ethoxy-2-nitrophenyl)-thioureido]-2-[3-thienyl] acetamide was isolated as a yellow solid, (16 mg, 43%) from 4-ethoxy-2-nitrophenylisothiocyanate (40 mg, 0.22 mmol) and N-(2-methylphenyl)₃-thienylglycinamide (20 mg, 0.08 mmol).

E48.2 N-(2-methylphenyl)-2-[3-(2-methoxy-5-nitrophenyl)-thioureido]-2-[3-thienyl]acetamide

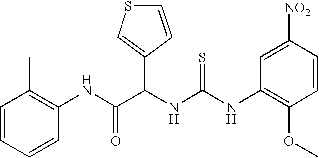

N-(2-methylphenyl)-2-[3-(2-methoxy-5-nitrophenyl)-th ioureido]-2-[3-thienyl] acetamide was isolated as a yellow solid, (31 mg, 85%) from 2-methoxy-5-nitrophenylisothiocyanate (27 mg, 0.13 mmol) and N-(2-methylphenyl)₃-thienylglycinamide (20 mg, 0.08 mmol).

E48.3 N-(2-methylphenyl)-2-[3-(2-trifluoromethylphenyl)-thioureido]-2-[3-thienyl]acetamide

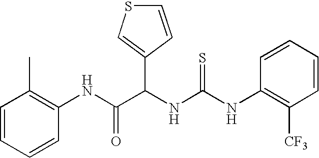

N-(2-methylphenyl)-2-[3-(2-trifluoromethylphenyl)-thioureido]-2-[3-thienyl] acetamide was isolated as a yellow solid, (30 mg, 83%) from 3-trifluoromethylphenylisothiocyanate (30 mg, 0.13 mmol) and N-(2-methylphenyl)₃-thienylglycinamide (20 mg, 0.08 mmol).

E48.4 N-(2-methylphenyl)-2-[3-(2-Nitrophenyl)-thioureido]-2-[3-thienyl] acetamide

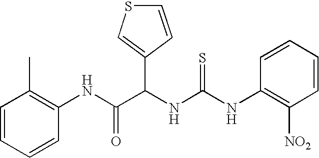

N-(2-methylphenyl)-2-[3-(2-Nitrophenyl)-thioureido]-2-[3-thienyl]acetamide was isolated as a yellow solid, (30 mg, 88%) from 2-nitrophenylisothiocyanate (21.6 mg, 0.12 mmol) and N-(2-methylphenyl)₃-thienylglycinamide (20 mg, 0.08 mmol).

### Example 49

C49 tert-butyl[1-(2-trifluoromethylphenylcarbamoyl)-1-phenyl-methyl]-carbamate

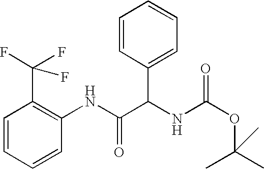

tert-butyl[1-(2-trifluoromethylphenylcarbamoyl)-1-phenyl-methyl]-carbamate was isolated as a white solid (104.0 mg, 35%) from N-tert butoxycarbonyl phenyl glycine (190.0 mg, 0.75 mmol) N-methylmorpholine (0.1 mL, 0.83 mmol), isobutylchloroformate (0.11 mL, 0.83 mmol) and 2-trifluoromethylaniline (134.0 mg, 0.83 mmol).

D49 2-Amino-N-(2-trifluoromethylphenyl)-2-phenyl acetamide

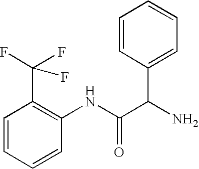

2-Amino-N-(2-trifluoromethylphenyl)-2-phenyl acetamide was isolated as a white solid (40.0 mg, 56%) from tert-butyl[1-(2-trifluoromethylphenylcarbamoyl)-1-phenyl-methyl]-carbamate (104.0 mg, 0.26 mmol).

E49.1 N-(2-trifluoromethyl phenyl)-2-[3-(2-nitrophenyl)-thioureido]-2-phenyl acetamide

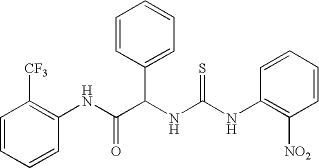

N-(2-trifluoromethylphenyl)-2-[3-(2-nitrophenyl)-thioureido]-2-phenyl acetamide was isolated as a yellow solid, (24 mg, 36%) from 2-nitrophenylisothiocyanate (37.8 mg, 0.21 mmol) and N-(2-trifluoromethylphenyl)phenylglycinamide (40 mg, 0.14 mmol).

### Example 50

E50.1 N-(2-ethylphenyl)-2-[3-(2-nitrophenyl)-thioureido]-2-phenyl acetamide

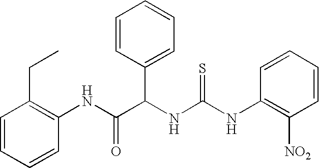

N-(2-ethylphenyl)-2-[3-(2-nitrophenyl)-thioureido]-2-phenyl acetamide was isolated as a yellow solid, (17.4 mg, 50%) from 2-nitrophenylisothiocyanate (21.6 mg, 0.12 mmol) and N-(2-ethylphenyl)phenylglycinamide (20 mg, 0.08 mmol).

### Example 51

A51 N-tert-butoxycarbonyl-D-leucine

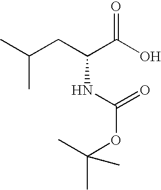

D-Leucine (2.0 g, 15.2 mmol) was mixed with potassium carbonate (8.4 g, 61.0 mmol) in water (50 mL) and acetone (10 ml) at ambient temperature. The reaction mixture was stirred for 5 minutes until the bubbling stopped. Then di(tert-butyl)dicarbonate (5.04 g, 22.9 mmoles) was added and the mixture was stirred overnight. The reaction mixture was diluted with water and extracted with ethyl acetate. The aqueous layer was acidified with 10% HCl to pH ˜3 and extracted with ethyl acetate. The combined organic layers were washed with brine, dried over anhydrous sodium sulfate, filtered and concentrated. The residue was triturated with hexane to give the titled compound as a white solid (3.3 g, 95%).

B51 tert-butyl-[1-(2,6-dimethylphenylcarbamoyl)-3-methyl-butyl]-carbamate

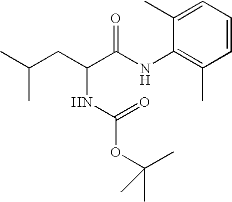

The titled compound was isolated as a white solid (1.65 g, 54%); from N-tert-butoxycarbonyl-D-leucine (2.11 g, 9.1 mmol), isobutyl chloroformate (1.3 mL, 10 mmol) and N-methylmorpholine (1.1 mL, 10 mmol) in THF (10 ml) at ˜40° C., followed by quenching with 2,6-dimethylaniline (1.3 mL, 10.9 mmol).

D51 2-amino-4-methyl-N-(2,6-dimethylphenyl)-pentanamide

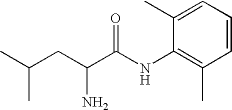

The titled compound was isolated as a colorless oil (1.10 g, 96%); from tert-butyl-[1-(2,6-dimethylphenylcarbamoyl)-3-methyl-butyl]-carbamate (1.65 g, 4.9 mmol) reacted with formic acid (15 mL) at 50° C. for one hour.

E51.1 N-(2.6-dimethylphenyl)-2-[3-(4-methoxycarbonylphenyl)-thioureido]-4-methylpentanamide

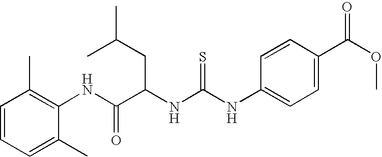

2-amino-4-methyl-N-(2,6-dimethylphenyl)-pentanamide (20.5 mg, 0.087 mmol) and 4-methoxycarbonylphenyl isothiocyanate (22 mg, 0.114 mmol) in dichloromethane (2 mL) were sealed in a vial and stirred at 50° C. for one hour. After cooling, hexane (2 mL) was added and then the precipitate was isolated by filtration and washed with 50% dichloromethane/hexane to give the titled compound as a white solid (25.3, 68%).

E51.2 N-(2,6-dimethyl phenyl)-2-[3-(2-trifluoromethylphenyl)-thioureido]-4-methylpentanamide

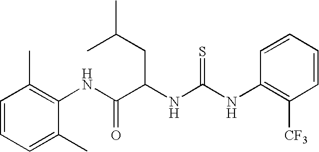

2-amino-4-methyl-N-(2,6-dimethylphenyl)-pentanamide (20.0 g, 0.085 mmoles) and 2-trifluoromethylphenyl isothiocyanate (22.6 mg, 0.111 mmoles) in dichloromethane (2 mL) were sealed in a vial and stirred at 50° C. for one hour. After cooling, the solvent was removed using a roto-evaporator and the residue was triturated with 5% ethyl acetate/hexane. The solid was isolated by filtration and washed with 5-10% ethyl acetate/hexane to give the titled compound as a white solid (16.7 mg, 45%).

E51.3 N-(2.6-dimethyl phenyl)-2-[3-(4-methoxy-2-nitrophenyl)-thioureido]-4-methylpentanamide

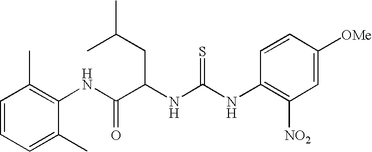

2-amino-4-methyl-N-(2,6-dimethylphenyl)-pentanamide (20.0 g, 0.085 mmoles) and 4-methoxy-2-nitrophenyl isothiocyanate (23.3 mg, 0.111 mmoles) in dichloromethane (2 mL) were sealed in a vial and stirred at 50° C. for one hour. After cooling, the solvent was removed using a roto-evaporator and the residue was triturated with 5% ethyl acetate/hexane. The solid was isolated by filtration and then purified by flash column chromatography eluted with 20% ethyl acetate/hexane to 100% ethyl acetate to give the titled compound as a yellow solid (21.3 mg, 56%).

E51.4 N-(2,6-dimethylphenyl)-2-[3-(4-ethoxy-2-nitrophenyl)-thioureido]-4-methylpentanamide

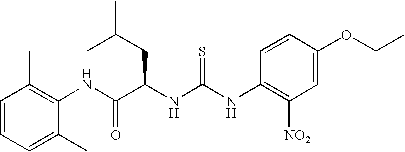

2-amino-4-methyl-N-(2,6-dimethylphenyl)-pentanamide (20.6 mg, 0.088 mmoles) and 4-ethoxy-2-nitrophenyl isothiocyanate (25.6 mg, 0.11 mmoles) in dichloromethane (2 mL) were stirred at 50° C. for one hour. After cooling, the solvent was removed using a roto-evaporator and the residue was triturated with 10% ethyl acetate/hexane. The solid was isolated by filtration and then washed with 10% ethyl acetate/hexane to give the titled compound as a yellow solid (27.4 g, 68%).

E51.5 N-(2.6-dimethylphenyl)-2-[3-(4-dimethylaminoethoxy-2-nitrophenyl)-thioureido]-4-methylpentanamide

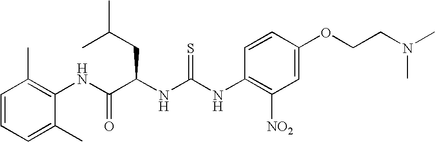

2-amino-4-methyl-N-(2,6-dimethylphenyl)-pentanamide (108 mg, 0.46 mmoles) and 4-dimethylaminoethoxy-2-nitrophenyl isothiocyanate (61.5 mg, 0.23 mmoles) in dichloromethane (mL) were stirred at room temperature overnight. The reaction was purified by flash column chromatography eluted with 10% ethyl acetate/hexane and then with 5-10% 2M ammonia in methanol/dichloromethane to give the titled compound as a yellow solid (30.0 mg, 26%).

E51.1* (R)-N-(2,6-dimethylphenyl)-2-[3-(4-methoxy-2-nitrophenyl)-thioureido]-4-methylpentanamide

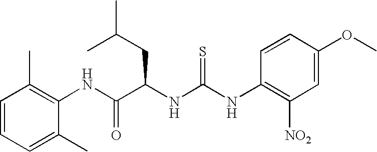

(R)-2-amino-4-methyl-N-(2,6-dimethylphenyl)-pentanamide (778 mg, 3.32 mmoles) and 4-methoxy-2-nitrophenyl isothiocyanate (907 mg, 4.31 mmoles) in dichloromethane (20 mL) were stirred at 50° C. for one hour. After cooling, the solvent was removed using a roto-evaporator and the residue was triturated with 10% ethyl acetate/hexane. The solid was isolated by filtration and then washed with 10% ethyl acetate/hexane to give the titled compound as a yellow solid (1.38 g, 94%).

### Example 52

B52 tert-butyl[1-(5-methoxy-2-methylphenylcarbamoyl)-1-phenyl-methyl]-carbamate

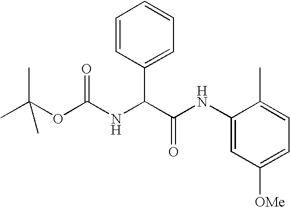

To a mixture of [(tert-butoxycarbonyl)amino](phenyl)acetic acid (300 mg, 1.19 mmol) and N-methylmorpholine (144 μL, 1.31 mmol) in THF (3 ml) at ˜−40° C. was added isobutyl chloroformate (170 μL, 1.31 mmol). After stirring for two hours, a mixture of 5-methoxy-2-methylaniline (197 mg, 1.43 mmol) and N-methylmorpholine (158 μL, 1.43 mmoles) in THF (2 mL) was added to the reaction and then left to stir overnight. The solvent was removed using a roto-evaporator and the residue was dissolved in dichloromethane. The organic layer was washed with water, 1 M sodium hydrosulphate and brine, dried over sodium sulfate and concentrated. The residue was triturated with hexane to give the titled compound as a white solid (435 mg, 99%).

D52 2-Amino-N-(5-methoxy-2-methylphenyl)-2-phenylacetamide

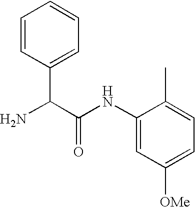

tert-butyl[1-(5-methoxy-2-methylphenylcarbamoyl)-1-phenyl-methyl]-carbamate (435 mg, 1.17 mmol) was mixed with 96% formic acid (3 mL) and heated to 50° C. for one hour. The reaction mixture was concentrated using a roto-evaporator and the residue was triturated with hexane to give the titled compound as a white solid product (263 mg, 83%).

E52.1 N-(5-methoxy-2-methylphenyl)-2-[3-(2-trifluoromethylphenyl)-thioureido]-2-phenyl acetamide

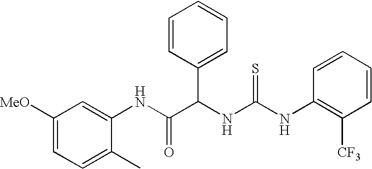

2-Amino-N-(2-methyl-5-methoxyphenyl)-2-phenylacetamide (20.0 g, 0.074 mmoles) and 2-trifluoromethylphenyl isothiocyanate (19.6 mg, 0.096 mmoles) in dichloromethane (2 mL) were sealed in a vial and stirred at 50° C. for one hour. After cooling, hexane (2 mL) was added and then the precipitate was isolated by filtration and washed with 50% dichloromethane/hexane to give the titled compound as a white solid (25.6 mg, 73%).

### Example 53

C53 1-tert-butyl-carbamoyl-1-[N-(2,6-dimethylphenyl)]-cyclopentane carboxamide

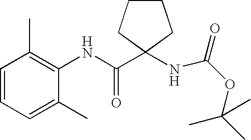

1-tert-butyl-carbamoyl-1-[N-(2,6-dimethylphenyl)]-cyclopentane carboxamide was isolated as a white solid (91.1 mg, 21%) from 1-[(tert)-butoxycarbonyl amino]-cyclopentane-1-carboxylic acid (300 mg, 1.31 mmol), N-methylmorpholine (0.16 mL, 1.44 mmol), isobutylchloroformate (0.19 mL, 1.44 mmol), 2,6-dimethylaniline (0.19 mL, 1.57 mmol) and N-methylmorpholine (0.17 mL, 1.57 mmol).

D53 1-amino-N-(2,6-dimethylphenyl)cyclopentane carboxamide

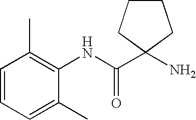

1-amino-N-(2,6-dimethylphenyl)cyclopentane carboxamide was isolated as a white solid (57.5 mg, 90%) from 1-tert-butyl-carbamoyl-1-[N-(2,6-dimethylphenyl)]-cyclopentane carboxamide (91.1 mg, 0.274 mmol) and formic acid (1.5 mL).

E53.1 N-(2,6-dimethylphenyl)-1-[3-(4-methoxycarbonylphenyl)-thioureido]-cyclopentane carboxamide

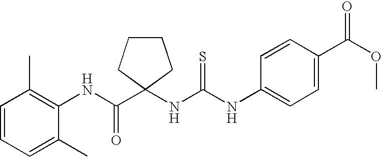

1-amino-N-(2,6-dimethylphenyl)-cyclopentane carboxamide (20.3 mg, 0.087 mmol) and 4-methoxycarbonylphenyl isothiocyanate (21.9 mg, 0.114 mmol) in dichloromethane (2 mL) were sealed in a vial and stirred at 50° C. for one hour. After cooling, hexane (2 mL) was added and then the precipitate was isolated by filtration and washed with 50% dichloromethane/hexane to give the titled compound as a white solid (17.8 mg, 48%).

E53.2 N-(2,6-dimethylphenyl)-1-[3-(4-methoxy-2-nitrophenyl)-thioureido]-cyclopentane carboxamide

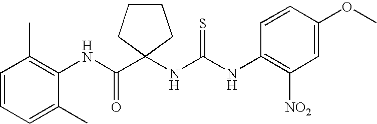

1-amino-N-(2,6-dimethylphenyl)-cyclopentane carboxamide (20.0 mg, 0.086 mmol) and 4-methoxycarbonylphenyl isothiocyanate (23.5 mg, 0.112 mmol) in dichloromethane (2 mL) were sealed in a vial and stirred at 50° C. for one hour. After cooling, hexane (2 mL) was added and then the precipitate was isolated by filtration and washed with 50% dichloromethane/hexane to give the titled compound as a yellow solid (11.9 mg, 31%).

### Example 54

E54.1 N-(2.6-dimethylphenyl)-2-[3-(4-methoxy-2-nitrophenyl)-thioureido]-2-(3,4-difluorophenyl)acetamide

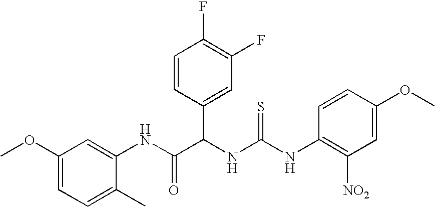

2-Amino-N-(2,6-dimethylphenyl)-2-(3,4-difluorophenyl)acetamide (20.0 g, 0.069 mmoles) and 4-methoxy-2-nitrophenyl isothiocyanate (18.8 mg, 0.090 mmoles) in dichloromethane (2 mL) were sealed in a vial and stirred at 50° C. for one hour. After cooling, the solvent was removed using a roto-evaporator and the residue was triturated with 5% ethyl acetate/hexane. The solid was isolated by filtration and first washed with 10% ethyl acetate/hexane and then with 50% dichloromethane/hexane to give the titled compound as a yellow solid (23.3 mg, 67%).

### Example 55

C55 tert-butyl[1-(4-isopropylphenylcarbamoyl)-1-phenyl-methyl]-carbamate

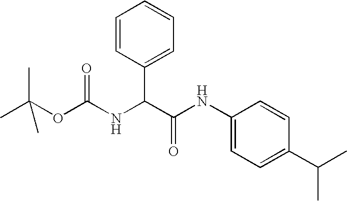

To the mixture of [(tert-butoxycarbonyl)amino](phenyl)acetic acid (500 mg, 2 mmoles) and N-methylmorpholine (241 μl, 2.2 mmoles) in THF (5 ml) at −40-50° C. was added isobutyl chloroformate (235 μl, 2.2 mmoles). After the reaction mixture was stirred for two hours, a mixture of 4-isopropylaniline (325 mg, 2.4 mmoles) and N-methylmorpholine (263.1 μl, 2.4 mmoles) were added. After the reaction mixture was stirred and left overnight, it was diluted with dichloromethane (20 ml) and washed with water (20 ml), 1M sodium hydrosulphate (20 ml×3) and brine (20 ml), dried with sodium sulfate, concentrated. The residue was triturated with hexanes to give white solid tert-butyl [1-(4-isopropylphenylcarbamoyl)-1-phenyl-methyl]-carbamate (577 mg, yield: 78.7%).

D55 2-Amino-N-(4-isopropylphenyl)-2-phenylacetamide formic acid salt

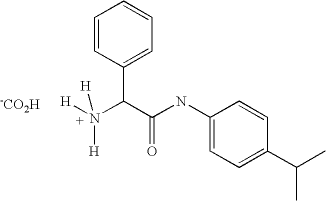

tert-butyl[1-(4-isopropylphenylcarbamoyl)-1-phenyl-methyl]-carbamate (500 mg, 1.36 mmoles) was mixed with 96% formic acid (5 ml) and heated to 60° C. for thirty minutes. The reaction mixture was concentrated by Rotavapor. The residue was triturated with hexanes and ether (1:1, 10 ml) to give white solid product 2-Amino-N-(4-isopropylphenyl)-2-phenylacetamide formic acid salt (426 mg, quantitative).

E55.1 N-(4-isopropylphenyl)-2-[3-(2-nitrophenyl)-thioureido]-2-phenyl acetamide

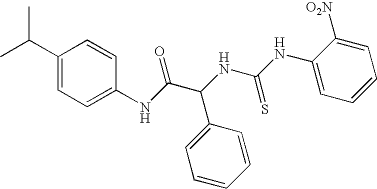

N-(4-isopropylphenyl)-2-[3-(2-nitrophenyl)-thioureido]-2-phenyl acetamide (18.2 mg, 81.2%); from 2-Amino-N-(4-isopropylphenyl)-2-phenylacetamide (15.6 mg, 0.05 mmoles), 2-nitrophenylthioisocyanate (10.8 mg, 0.06 mmoles) and triethylamine (15.2 mg, 0.15 mmoles) in dichloromethane (1 ml) at ambient temperature overnight.

### Example 56

C56 tert-butyl[1-(4-nitrophenylcarbamoyl)-1-phenyl-methyl]-carbamate

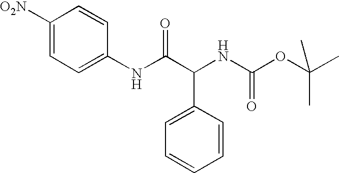

tert-butyl[1-(4-nitrophenylcarbamoyl)-1-phenyl-methyl]-carbamate (280 mg, 37.8%); from [(tert-butoxycarbonyl)amino](phenyl)acetic acid (500 mg, 2 mmoles) reacted with isobutyl chloroformate (235 μl, 2.2 mmoles) and N-methylmorpholine (241 μl, 2.2 mmoles) in THF (5 ml) at −40˜−50° C., followed by being quenched with 4-nitroaniline (331,2 mg, 2.4 mmoles).

D56 2-Amino-N-(4-nitrophenyl)-2-phenylacetamide

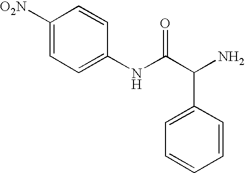

2-Amino-N-(4-nitrophenyl)-2-phenylacetamide (114.5 mg, 55.9%); from tert-butyl [1-(4-nitrophenylcarbamoyl)-1-phenyl-methyl]-carbamate (280 mg, 0.754 mmoles) reacted with formic acid (2 ml) at 60° C. for thirty minutes.

E56.1 N-(4-nitrophenyl)-2-[3-(2-nitrophenyl)-thioureido]-2-phenyl acetamide

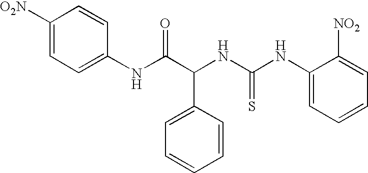

N-(4-nitrophenyl)-2-[3-(2-nitrophenyl)-thioureido]-2-phenyl acetamide (2.4 mg, 10.6%); from 2-Amino-N-(4-nitrophenyl)-2-phenylacetamide (13.6 mg, 0.05 mmoles) reacted with 2-nitrophenylthioisocyanate (10.8 mg, 0.06 mmoles) and triethylamine (15.2 mg, 0.15 mmoles) in dichloromethane (1 ml) at 60° C. overnight.

### Example 57

C57 tert-butyl[1-(2,5-dimethylcarbamoyl)-1-(3,4-difluorophenyl) methyl]carbamate

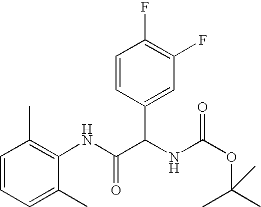

tert-butyl[1-(2,5-dimethylcarbamoyl)-1-(3,4-difluorophenyl)methyl]carbamate was isolated as a white solid (281 mg, 83%) from N-tert-butoxycarbonyl 3,4-difluorophenyl glycine (250.0 mg, 0.87 mmol), n-methylmorpholine (0.11 mL, 0.96 mmol), isobutylchloroformate (0.12 mL, 0.96 mmol), 2,6-dimethylaniline (0.13 mL, 1.04 mmol) and N-methylmorpholine (0.17 mL, 115 mmol).

D57 2-Amino-N-(2,6-dimethylphenyl)-2-3,4-difluorophenylacetamide

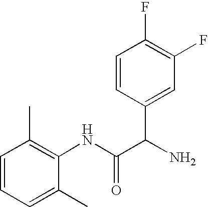

2-Amino-N-(2,6-dimethylphenyl)-2-3,4-difluorophenylacetamide was isolated as a white solid (170.0 mg, 81%) from tert-butyl[1-(2,5-dimethylcarbamoyl)-1-(3,4-difluorophenyl)methyl]carbamate (281.0 mg, 0.72 mmol) and formic acid (3 mL).

E57.1 N-(2.6-dimethylphenyl)-2-[3-(2-trifluoromethylphenyl)-thioureido]-2-(3.4-difluorophenyl)acetamide

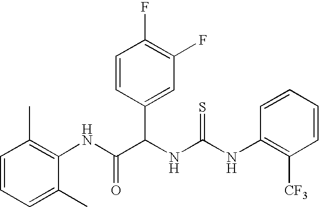

2-Amino-N-(2,6-dimethylphenyl)-2-(3,4-difluorophenyl)acetamide (20.0 g, 0.069 mmoles) and 2-trifluoromethylphenyl isothiocyanate (18.2 mg, 0.090 mmoles) in dichloromethane (2 mL) were sealed in a vial and stirred at 50° C. for one hour. After cooling, the solvent was removed using a roto-evaporator and the residue was triturated with 5% ethyl acetate/hexane. The solid was isolated by filtration and washed with 5-10% ethyl acetate/hexane to give the titled compound as a white solid (21.2 mg, 62%).

### Example 58

### Assay of Transport via GlyT-2 Transporters

This example illustrates a method for the measurement of glycine uptake by transfected cultured cells.

Cells stably transfected with human GlyT-2 (the homolog of the rat GlyT-2 described by Liu et al., J. Biological Chemistry, 268, 1993:22802-22808) were washed twice with HEPES buffered saline (HBS). The cells were then incubated ten minutes at 37° C., after which a solution containing 50 nM [³H]glycine (17.5 Ci/mmol) and either (a) no potential competitor, (b) 10 nM glycine or (c) a concentration of a candidate drug. A range of concentrations of the candidate drug was used to generate data for calculating the concentration resulting in 50% of the effect (e.g., the IC₅₀'s which are the concentration of drug inhibiting glycine uptake by 50%). The cells were then incubated another ten minutes at 37° C., after which the cells were aspirated and washed three times with ice-cold HBS. The cells were solubilized in scintillant, shaken for thirty minutes, and the radioactivity in the cells was counted using a scintillation counter. Data were compared between the same cells contacted and not contacted by the candidate agent.

The compounds of the present invention were active as GlyT-2 inhibitors. The following table provides examples of the glycine uptake IC50 values for representative compounds of the invention.

| Experiment Number | GlyT1 uptake IC50 (nM) |
|---|---|
| E4.2 | 77.42 |
| E4.4 | 83.8075 |
| E4.3 | 171.8167 |
| E33.5 | 14.2206 |
| E33.6 | 34.3767 |
| E33.8 | 114.7548 |
| E28.1 | 52.2478 |
| E29.1 | 22.799 |
| E51.3 | 111.867 |
| E33.3 | 41.482 |
| E51.1* | 90.771 |
| E33.1 | 49.1594 |
| E33.2 | 47.9775 |
| E33.3 | 225.2475 |
| E33.4 | 182.375 |

We claim:

1. A compound of the Formula 1: 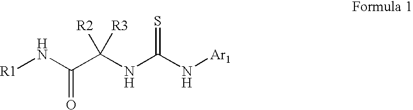
wherein;
R₁ is selected from the group consisting of aryl, heteroaryl, cylcloalkyl and heterocycloalkyl;
wherein R₁ is optionally substituted with one or more substituents Rₐ;
wherein Rₐ is selected from the group consisting of alkyl, alkoxy, halo, cyano, alkanoyl, haloalkyl, thioalkyl, nitro, aryl, heteroaryl, aralkyl, heteroaralkyl and —(R₇)ₙNR₈R₉
wherein R₇ is selected from alkyl, alkoxy, and oxyalkyl, R₈ and R₉ can be independently selected from H, and alkyl, or R₈ and R₉ can join together such that NR₈R₉ form a 5 or 6-member heterocyclic ring, and n is selected from 0, 1, 2 and 3)
wherein the substituents(s) Rₐ is optionally further substituted with one or more substituents are selected from the group consisting of alkyl, alkoxy, halo, cyano, alkanoyl, haloalkyl, thioalkyl, nitro, and —(R₇)ₙN R₈R₉, wherein R₇, R₈, R₉ and n are as defined above, and
R₂ and R₃ are:
a) independently selected from the group consisting of H, alkyl, aralkyl optionally substituted aryl, optionally substituted heteroaryl and optionally substituted, saturated or unsaturated, 5- or 6-membered, homocyclic or heterocyclic rings wherein the optional substituent may be selected from the group consisting of H, alkyl, alkoxy, and halo; or
b) joined together to form a 3, 4, 5, 6 or 7 member spirocyclic ring, and
Ar₁ is aryl and;
Ar₁ is optionally substituted with one or more substituents R_{b}; wherein R_{b} is selected from the group consisting of: alkyl, alkoxy, halo, haloalkyl, nitro, —(R₇)ₙNR₈R₉, alkanoyl, aryl, heteroaryl, —O(CH₂)ₘNR₁₀R₁₁ and —SO₂—NR₁₀R₁₁ (wherein R₇ is selected from alkyl, alkoxy; and oxyalkyl, R₈ and R₉ can be independently selected from H, and alkyl, or R₈ and R₉ can join together such that NR₈R₉ form a 5 or 6-member heterocyclic ring, and n is selected from 0, 1, 2 and 3) and the groups R₁₀ and R₁₁ can be independently selected from H, or alkyl, or groups R₁₀ and R₁₁ can join together such that NR₁₀ R₁₁ form a 5 or 6-member ring, and m is selected from 1, 2, 3, 4 and 5);
wherein the substituent(s) R_{b} are optionally further substituted with one or more substituents selected from the group consisting of alkyl, alkoxy, halo, cyano, alkanoyl, haloalkyl, thioalkyl, nitro and —(R₇)ₙNR₈R₉ (wherein R₇, R₈, R₉ and n are as described above).
wherein when Ar₁ is phenyl then
a) Ar₁ has a substituent R_{b} at the 2-position wherein the substituent is selected from the group consisting of nitro, haloalkyl, cyano, —C(O)R₁₂—C(O)OR₁₂, —C(O)NR₁₂R₁₂, —S(O)R₁₂, —S(O)₂R₁₂, and —S(O)₂NR₁₂R₁₃ (wherein R₁₂ and R₁₃ are independently selected from H and alkyl) or
b) Ar₁ has an alkanoyl substituent at the 4-position,
and a salt solvate or hydrate thereof.

2. A compound of claim 1 wherein Ar₁ is selected from the group consisting of phenyl and naphthyl.

3. A compound of claim 2 wherein Ar₁ is naphthyl.

4. A compound of claim 2 wherein Ar₁ is 4-acetylphenyl.

5. A compound of claim 2 wherein Ar₁ is phenyl, and there is a substituent R_{b} at the 2-position and R_{b} is selected from the group consisting of nitro, trifluoromethyl and —SO₂—NR₁₀R₁₁.

6. A compound of claim 5 wherein the substituent R_{b} at the 2-position is nitro.

7. A compound of claim 5 with a second substituent R_{b} at the 4-position selected from the group consisting of: methoxy; ethoxy; propoxy; —O(CH₂)ₘNR₁₀R₁₁ and acetyl.

8. A compound of claim 7 wherein Ar₁ is 2-nitro-4-methoxyphenyl.

9. A compound of claim 1 wherein R₁ is selected from the group consisting of: phenyl; naphthyl; tetrahydronaphthyl; and pyridyl.

10. A compound of claim 9 wherein R₁ is pyridyl.

11. A compound of claim 9 wherein R₁ is naphthyl or tetrahydronaphthyl.

12. A compound of claim 9 wherein R₁ is phenyl.

13. A compound of claim 12 wherein R₁ is substituted with one or more substituents Rₐ, wherein Rₐ is selected from the group consisting of: alkyl; alkoxy; halo; cyano; thioalkyl; nitro; alkanoyl; haloalkyl; acetyl; piperazinyl.

14. A compound of claim 13 wherein the substituent(s) Rₐ are independently selected from the group consisting of: methyl; ethyl; isopropyl; chloro; fluoro; trifluoromethyl; thiomethyl; cyano; nitro; methoxy and piperazinyl.

15. A compound of claim 14 wherein there is one substituent Rₐ.

16. A compound of claim 15 wherein the substituent Rₐ is located at the 2-position of the phenyl ring R₁.

17. A compound of claim 16 wherein Rₐ is methyl.

18. A compound of claim 14 wherein there are two substituents Rₐ.

19. A compound of claim 18 wherein the two substituents Rₐ are located at the 2-position and the 6-position.

20. A compound of claim 19 wherein one of the substituents Rₐ is methyl, and the second substituent Rₐ is selected from the group consisting of: methyl, and ethyl.

21. A compound of claim 20 wherein the second substituent Rₐ is methyl.

22. A compound as defined in claim 1 wherein R₂ and R₃ are independently selected from H, alkyl, aralkyl, and optionally substituted, saturated or unsaturated, 5 or 6-member homocyclic or heterocyclic rings; or R₂ and R₃ are joined together to form a 3, 5 or 6 member spirocyclic ring.

23. A compound as described in claim 22 wherein R₂ and R₃ are selected independently from H, methyl, isopropyl, t-Butyl, sec-Butyl, cyclohexyl, phenyl, benzyl, 3-thiophene.

24. A compound as described in claim 22 wherein R₂ and R₃ join together to form a 3, 5, or 6-member spirocyclic ring.

25. A compound from claim 1 selected from the group consisting of:
N-(2-methyl phenyl)-2-[3-(4-ethoxy-2-nitrophenyl)-thioureido]-2-phenyl acetamide (E4.3);
N-(2,6-dimethylphenyl)-2-[3-(4-methoxy-2-nitrophenyl)-thioureido]-2-phenyl acetamide (E33.6);
N-(2-methylphenyl)-2-[3-(2-nitrophenyl)-thioureido]-2-phenyl acetamide (E4.2)
N-(2-methylphenyl)-2-[3-(2-nitro-4-methoxyphenyl)-thioureido]-2-phenyl acetamide (E4.4);
N-(2,6-dimethylphenyl)-2-[3-(2-trifluoromethylphenyl)-thioureido]-2-phenyl acetamide (E33.7);
N-(2,6-dimethylphenyl)-2-[3-(4-N,N-dimethylaminoethoxy-2-trifluoromethylphenyl)-thioureido]-2-phenyl acetamide (E33.8);
N-(2-isopropyl-6-methylphenyl)-2-[3-(4-methoxy-2-nitrophenyl)-thioureido]-2-phenyl acetamide (E28.1);
N-(2-chloro-6-methylphenyl)-2-[3-(4-methoxy-2-nitrophenyl)-thioureido]-2-phenyl acetamide (E29.1);
N-(2,6-dimethylphenyl)-2-[3-(4-methoxy-2-nitrophenyl)-thioureido]-4-methylpentanamide (E51.3);
N-(2,6-dimethylphenyl)-2-[3-(4-(2-N,N-dimethylamino)ethoxy-2-nitrophenyl)-thioureido]-2-phenyl acetamide (E33.4);
(R)-N-(2,6-dimethylphenyl)-2-[3-(4-methoxy-2-nitrophenyl)-thioureido]-4-methylpentanamide (E51.1*);
N-(2,6-dimethylphenyl)-2-[3-(4-ethoxy-2-nitrophenyl)-thioureido]-2-phenyl acetamide (E33.1);
N-(2,6-dimethylphenyl)-2-[3-(2-N,N-dimethylsulphonamidophenyl)-thioureido]-2-phenyl acetamide (E33.2);
N-(2,6-dimethylphenyl)-2-[3-(2-N-methylpiperizinylsulphonamidophenyl)-thioureido]-2-phenyl acetamide (E33.3);
and N-(2,6-dimethyl phenyl)-2-[3-(4-(2-N,N-dimethylamino)sulphonamide-2-nitro-thioureido]-2-phenyl acetamide (E33.5).

26. A pharmaceutical composition comprising a therapeutically effective amount of a compound of claim 1 and a pharmaceutically acceptable carrier.

27. A method for treating a patient having a medical condition for which a glycine transport inhibitor is indicated, comprising the step of administering to a patient a pharmaceutical composition as described in claim 26.

28. A method according to claim 27 wherein the medical condition is pain or spasticity.
